SEQUENCE LISTING (30 sequences)

SEQ ID NO: 1 - protein, 119 aa, synthetic construct
  REGION 1..119: note = VH of antibody 13F11
EVQLVESGGGLVQPGRSLKLSCAASRFTFSNYDMAWVRQAPTKGLEWVASISPSNVNTYYRDSVKGRFTVSRDNAKSSLYLQMDSLRSEDTATYYCARLVHSFWYFDFWGPGTMVTVSS

SEQ ID NO: 2 - protein, 115 aa, synthetic construct
  REGION 1..115: note = VL of antibody 13F11
DIVMTQTPSSQAVSAGEKVTMRCKSSQSLLYSKNKKNYLAWYQQKPGQSPKLLIYWASTRESGVPDRFLGSGSGTDFTLTISSVQAEDLAVYYCQQYYKFPPTFGGGTKLELKRA

SEQ ID NO: 3 - protein, 119 aa, synthetic construct
  REGION 1..119: note = VH of antibody 23A7
EVQLVESGGDLVQPGRSLKLSCAASGFTFSNYDMAWVRQAPTQGLEWVASISGSGGTTYYRDSVKGRFTVSRDNAKSTLYLQMDGLRSEDTATYFCARHPPSFWYFDFWGPGSMVTVSS

SEQ ID NO: 4 - protein, 115 aa, synthetic construct
  REGION 1..115: note = VL of antibody 23A7
DIVMTQTPSSQAVSAGEKVTMSCKSGQSLLFNKNKKDYLAWYQRKPGQSPKLLIYWASTRESGVPDRFIGGGSGTDFTLTISSVQAEDLAVYYCQQYYRFPPTFGHGTKLELKRA

SEQ ID NO: 5 - protein, 119 aa, synthetic construct
  REGION 1..119: note = VH of antibody 18A4 and antibody 18A4-2
EVQLVESGGGLVQPGRSLKLSCAASGFTFSNYDMAWVRQAPTKGLEWVASISPSGGSIYYRDSVKGRFTVSRNNAKSSLYLQMDSLRSEDTATYYCARLTTYFWYFDFWGPGTMVTVSS

SEQ ID NO: 6 - protein, 109 aa, synthetic construct
  REGION 1..109: note = VL of antibody 18A4
DIQMTQSPSFLSASVGDRVTINCKASQNSNKYLNWYQQKLGEAPKLLIYNTNNLQTGIPSRFSGSGSGTDYTLTISSLQPEDVATYFCLQHSSRMDTFGAGTKLELKRA

SEQ ID NO: 7 - protein, 5 aa, synthetic construct
  REGION 1..5: note = VH-CDR1 of antibody 13F11
NYDMA

SEQ ID NO: 8 - protein, 17 aa, synthetic construct
  REGION 1..17: note = VH-CDR2 of antibody 13F11
SISPSNVNTYYRDSVKG

SEQ ID NO: 9 - protein, 10 aa, synthetic construct
  REGION 1..10: note = VH-CDR3 of antibody 13F11
LVHSFWYFDF

SEQ ID NO: 10 - protein, 17 aa, synthetic construct
  REGION 1..17: note = VL-CDR1 of antibody 13F11
KSSQSLLYSKNKKNYLA

SEQ ID NO: 11 - protein, 7 aa, synthetic construct
  REGION 1..7: note = VL-CDR2 of antibody 13F11
WASTRES

SEQ ID NO: 12 - protein, 9 aa, synthetic construct
  REGION 1..9: note = VL-CDR3 of antibody 13F11
QQYYKFPPT

SEQ ID NO: 13 - protein, 5 aa, synthetic construct
  REGION 1..5: note = VH-CDR1 of antibody 23A7
NYDMA

SEQ ID NO: 14 - protein, 17 aa, synthetic construct
  REGION 1..17: note = VH-CDR2 of antibody 23A7
SISGSGGTTYYRDSVKG

SEQ ID NO: 15 - protein, 10 aa, synthetic construct
  REGION 1..10: note = VH-CDR3 of antibody 23A7
HPPSFWYFDF

SEQ ID NO: 16 - protein, 17 aa, synthetic construct
  REGION 1..17: note = VL-CDR1 of antibody 23A7
KSGQSLLFNKNKKDYLA

SEQ ID NO: 17 - protein, 7 aa, synthetic construct
  REGION 1..7: note = VL-CDR2 of antibody 23A7
WASTRES

SEQ ID NO: 18 - protein, 9 aa, synthetic construct
  REGION 1..9: note = VL-CDR3 of antibody 23A7
QQYYRFPPT

SEQ ID NO: 19 - protein, 5 aa, synthetic construct
  REGION 1..5: note = VH-CDR1 of antibody 18A4 and of antibody 18A4-2
NYDMA

SEQ ID NO: 20 - protein, 17 aa, synthetic construct
  REGION 1..17: note = VH-CDR2 of antibody 18A4 and of antibody 18A4-2
SISPSGGSIYYRDSVKG

SEQ ID NO: 21 - protein, 10 aa, synthetic construct
  REGION 1..10: note = VH-CDR1 of antibody 18A4 and of antibody 18A4-2
LTTYFWYFDF

SEQ ID NO: 22 - protein, 11 aa, synthetic construct
  REGION 1..11: note = VL-CDR1 of antibody 18A4
KASQNSNKYLN

SEQ ID NO: 23 - protein, 7 aa, synthetic construct
  REGION 1..7: note = VL-CDR2 of antibody 18A4
NTNNLQT

SEQ ID NO: 24 - protein, 9 aa, synthetic construct
  REGION 1..9: note = VL-CDR3 of antibody 18A4
LQHSSRMDT

SEQ ID NO: 25 - protein, 2752 aa, Homo sapiens
MYNGIGLPTPRGSGTNGYVQRNLSLVRGRRGERPDYKGEEELRRLEAALVKRPNPDILDHERKRRVELRCLELEEMMEEQGYEEQQIQEKVATFRLMLLEKDVNPGGKEETPGQRPAVTETHQLAELNEKKNERLRAAFGISDSYVDGSSFDPQRRAREAKQPAPEPPKPYSLVRESSSSRSPTPKQKKKKKKKDRGRRSESSSPRRERKKSSKKKKHRSESESKKRKHRSPTPKSKRKSKDKKRKRSRSTTPAPKSRRAHRSTSADSASSSDTSRSRSRSAAAKTHTTALAGRSPSPASGRRGEGDAPFSEPGTTSTQRPSSPETATKQPSSPYEDKDKDKKEKSATRPSPSPERSSTGPEPPAPTPLLAERHGGSPQPLATTPLSQEPVNPPSEASPTRDRSPPKSPEKLPQSSSSESSPPSPQPTKVSRHASSSPESPKPAPAPGSHREISSSPTSKNRSHGRAKRDKSHSHTPSRRMGRSRSPATAKRGRSRSRTPTKRGHSRSRSPQWRRSRSAQRWGRSRSPQRRGRSRSPQRPGWSRSRNTQRRGRSRSARRGRSHSRSPATRGRSRSRTPARRGRSRSRTPARRRSRSRTPTRRRSRSRTPARRGRSRSRTPARRRSRTRSPVRRRSRSRSPARRSGRSRSRTPARRGRSRSRTPARRGRSRSRTPARRSGRSRSRTPARRGRSRSRTPRRGRSRSRSLVRRGRSHSRTPQRRGRSGSSSERKNKSRTSQRRSRSNSSPEMKKSRISSRRSRSLSSPRSKAKSRLSLRRSLSGSSPCPKQKSQTPPRRSRSGSSQPKAKSRTPPRRSRSSSSPPPKQKSKTPSRQSHSSSSPHPKVKSGTPPRQGSITSPQANEQSVTPQRRSCFESSPDPELKSRTPSRHSCSGSSPPRVKSSTPPRQSPSRSSSPQPKVKAIISPRQRSHSGSSSPSPSRVTSRTTPRRSRSVSPCSNVESRLLPRYSHSGSSSPDTKVKPETPPRQSHSGSISPYPKVKAQTPPGPSLSGSKSPCPQEKSKDSLVQSCPGSLSLCAGVKSSTPPGESYFGVSSLQLKGQSQTSPDHRSDTSSPEVRQSHSESPSLQSKSQTSPKGGRSRSSSPVTELASRSPIRQDRGEFSASPMLKSGMSPEQSRFQSDSSSYPTVDSNSLLGQSRLETAESKEKMALPPQEDATASPPRQKDKFSPFPVQDRPESSLVFKDTLRTPPRERSGAGSSPETKEQNSALPTSSQDEELMEVVEKSEEPAGQILSHLSSELKEMSTSNFESSPEVEERPAVSLTLDQSQSQASLEAVEVPSMASSWGGPHFSPEHKELSNSPLRENSFGSPLEFRNSGPLGTEMNTGFSSEVKEDLNGPFLNQLETDPSLDMKEQSTRSSGHSSSELSPDAVEKAGMSSNQSISSPVLDAVPRTPSRERSSSASSPEMKDGLPRTPSRRSRSGSSPGLRDGSGTPSRHSLSGSSPGMKDIPRTPSRGRSECDSSPEPKALPQTPRPRSRSPSSPELNNKCLTPQRERSGSESSVDQKTVARTPLGQRSRSGSSQELDVKPSASPQERSESDSSPDSKAKTRTPLRQRSRSGSSPEVDSKSRLSPRRSRSGSSPEVKDKPRAAPRAQSGSDSSPEPKAPAPRALPRRSRSGSSSKGRGPSPEGSSSTESSPEHPPKSRTARRGSRSSPEPKTKSRTPPRRRSSRSSPELTRKARLSRRSRSASSSPETRSRTPPRHRRSPSVSSPEPAEKSRSSRRRRSASSPRTKTTSRRGRSPSPKPRGLQRSRSRSRREKTRTTRRRDRSGSSQSTSRRRQRSRSRSRVTRRRRGGSGYHSRSPARQESSRTSSRRRRGRSRTPPTSRKRSRSRTSPAPWKRSRSRASPATHRRSRSRTPLISRRRSRSRTSPVSRRRSRSRTSVTRRRSRSRASPVSRRRSRSRTPPVTRRRSRSRTPTTRRRSRSRTPPVTRRRSRSRTPPVTRRRSRSRTSPITRRRSRSRTSPVTRRRSRSRTSPVTRRRSRSRTSPVTRRRSRSRTPPAIRRRSRSRTPLLPRKRSRSRSPLAIRRRSRSRTPRTARGKRSLTRSPPAIRRRSASGSSSDRSRSATPPATRNHSGSRTPPVALNSSRMSCFSRPSMSPTPLDRCRSPGMLEPLGSSRTPMSVLQQAGGSMMDGPGPRIPDHQRTSVPENHAQSRIALALTAISLGTARPPPSMSAAGLAARMSQVPAPVPLMSLRTAPAANLASRIPAASAAAMNLASARTPAIPTAVNLADSRTPAAAAAMNLASPRTAVAPSAVNLADPRTPTAPAVNLAGARTPAALAALSLTGSGTPPTAANYPSSSRTPQAPASANLVGPRSAHATAPVNIAGSRTAAALAPASLTSARMAPALSGANLTSPRVPLSAYERVSGRTSPPLLDRARSRTPPSAPSQSRMTSERAPSPSSRMGQAPSQSLLPPAQDQPRSPVPSAFSDQSRCLIAQTTPVAGSQSLSSGAVATTTSSAGDHNGMLSVPAPGVPHSDVGEPPASTGAQQPSALAALQPAKERRSSSSSSSSSSSSSSSSSSSSSSSSSGSSSSDSEGSSLPVQPEVALKRVPSPTPAPKEAVREGRPPEPTPAKRKRRSSSSSSSSSSSSSSSSSSSSSSSSSSSSSSSSSSSSSSSSSSPSPAKPGPQALPKPASPKKPPPGERRSRSPRKPIDSLRDSRSLSYSPVERRRPSPQPSPRDQQSSSSERGSRRGQRGDSRSPSHKRRRETPSPRPMRHRSSRSP

SEQ ID NO: 26 - protein, 113 aa, synthetic construct
  REGION 1..113: note = VL of antibody 18A4-2
DIVMTQTPSSQAVSAGEKVTMSCKSSQSLLYSENKKNYLAWYQQKPGQSPKLLIYWASTRESGVPDRFIGSGSGTDFTLTISSVQAEDLAVYYCQQYYKFPPTFGSGTKLEIK

SEQ ID NO: 27 - protein, 17 aa, synthetic construct
  REGION 1..17: note = VL-CDR1 of antibody 18A4-2
KSSQSLLYSENKKNYLA

SEQ ID NO: 28 - protein, 7 aa, synthetic construct
  REGION 1..7: note = VL-CDR2 of antibody 18A4-2
WASTRES

SEQ ID NO: 29 - protein, 9 aa, synthetic construct
  REGION 1..9: note = VL-CDR3 of antibody 18A4-2
QQYYKFPPT

SEQ ID NO: 30 - protein, 262 aa, synthetic construct
  REGION 1..262: note = fragment tr04
RSRTSPVSRRRSRSRTSVTRRRSRSRASPVSRRRSRSRTPPVTRRRSRSRTPTTRRRSRSRTPPVTRRRSRSRTPPVTRRRSRSRTSPITRRRSRSRTSPVTRRRSRSRTSPVTRRRSRSRTSPVTRRRSRSRTPPAIRRRSRSRTPLLPRKRSRSRSPLAIRRRSRSRTPRTARGKRSLTRSPPAIRRRSASGSSSDRSRSATPPATRNHSGSRTPPVALNSSRMSCFSRPSMSPTPLDRCRSPGMLEPLGSSRTPMSVLQ

(19) United States
(12) Patent Application Publication
(10) Pub. No.: US 2026/0308237 A1
(43) Pub. Date: Oct. 8, 2026

(54) COMPOSITION COMPRISING AN ANTIBODY WHICH BINDS TO HUMAN SRRM2 PRESENT ON THE CELL SURFACE OF A TARGET CELL

(71) Applicants: Helmholtz Zentrum München - Deutsches Forschungszentrum Für Gesundheit Und Umwelt (GmbH), Neuherberg (DE); Eximmium Biotechnologies GmbH, Munich (DE); Klinikum der Universitaet Muenchen, Munich (DE)

(72) Inventors: Reinhard Zeidler, Olching (DE); Kathrin Gaertner, Munich (DE)

(21) Appl. No.: 18/846,632

(22) PCT Filed: Mar. 14, 2023
(86) PCT No.: PCT/EP2023/056397

§ 371 Date: Sep. 12, 2024

(30) Foreign Application Priority Data
Mar. 14, 2022 (EP) 22161839.0

(51) Int. Cl. C07K 16/18; A61K 38/17; A61K 47/68; A61P 35/00

(52) U.S. Cl. CPC C07K 16/18; A61K 38/1774; A61K 47/6803; A61K 47/6843; A61P 35/00

(57) ABSTRACT

The present invention provides a pharmaceutical composition comprising an antibody, which binds to human SRRM2 present on the cell surface of a target cell and optionally a pharmaceutically acceptable carrier, diluent or excipient. The present invention further provides said pharmaceutical composition for use in a method for the treatment of cancer in a human subject. Further is provided a method for determining whether a human subject may suffer from cancer, comprising determining in a sample obtained from said human subject whether SRRM2 is present on the cell surface of cells comprised by said sample. Additionally, an antibody, which binds to human SRRM2 present on the cell surface of a target cell is provided and an antibody, which binds to human SRRM2 present on the cell surface of a target cell for use in a method of killing said target cell having human SRRM2 present on the cell surface.

### TECHNICAL FIELD OF THE INVENTION

The present invention provides a pharmaceutical composition comprising an antibody, which binds to human SRRM2 present on the cell surface of a target cell and optionally a pharmaceutically acceptable carrier, diluent or excipient. The present invention further provides said pharmaceutical composition for use in a method for the treatment of cancer in a human subject. Further is provided a method for determining whether a human subject may suffer from cancer, comprising determining in a sample obtained from said human subject whether SRRM2 is present on the cell surface of cells comprised by said sample. Additionally, an antibody which binds to human SRRM2 present on the cell surface of a target cell is provided. The present invention further provides an antibody, which binds to human SRRM2 present on the cell surface of a target cell for use in a method of killing said target cell having SRRM2 present on the cell surface. Further, a method of killing a target cell having SRRM2 present on the cell surface of said target cell, comprising administering an antibody, which binds to human SRRM2 present on the cell surface of said target cell is provided. The present invention further relates to an antibody, which binds to human SRRM2 present on the cell surface of a target cell for use in a method for the treatment of cancer in a subject, comprising, prior to the treatment of cancer in said subject, determining whether a target cell of said subject has SRRM2 present on the cell surface.

### BACKGROUND ART

In 2015, about 90.5 million people had cancer. As of 2019, about 18 million new cases occur annually. Annually, it caused about 8.8 million deaths (15.7% of deaths). The most common types of cancer in males are lung cancer, prostate cancer, colorectal cancer, and stomach cancer. In females, the most common types are breast cancer, colorectal cancer, lung cancer, and cervical cancer. If skin cancer other than melanoma were included in total new cancer cases each year, it would account for around 40% of cases. In children, acute lymphoblastic leukemia and brain tumors are most common, except in Africa, where non-Hodgkin lymphoma occurs more often. In 2012, about 165,000 children under 15 years of age were diagnosed with cancer. The risk of cancer increases significantly with age, and many cancers occur more commonly in developed countries. Rates are increasing as more people live to an old age and as lifestyle changes occur in the developing world. The financial costs of cancer were estimated at 1.16 trillion USD per year as of 2010.

Accordingly, there is still need for further cancer biomarkers that allows discrimination of healthy, non-cancer cells from cancer cells. The underlying technical problem of this application is thus the provision of further cancer biomarkers.

### SUMMARY OF THE INVENTION

The technical problem is solved by the subject-matter as defined in the claims. The inventors surprisingly found that Serine/arginine repetitive matrix protein 2 (SRRM2; UniProt Q9UQ35) is expressed on cancer cells, but not in healthy, non-cancer cells. Thus, the expression of SRRM2 on the cell surface of cells, e.g., obtained from a patient, is a biomarker for cancer. Furthermore, the expression on the surface of solely cancer cells allows novel cancer-specific therapies.

Accordingly, the present invention provides a pharmaceutical composition comprising an antibody which binds to human SRRM2 present on the cell surface of a target cell and optionally a pharmaceutically acceptable carrier, diluent or excipient.

The target cell preferably is a cancer cell.

The antibody, which binds to human SRRM2, may have cytotoxic activity. The antibody, which binds to human SRRM2 may have ADCC or CDC. The antibody, which binds to human SRRM2 may be conjugated with a cytotoxic substance.

The antibody, which binds to human SRRM2 may be part of a chimeric antigen receptor (CAR). The CAR may be comprised by a T cell, NK cell, NK-T cell or macrophage.

SRRM2, preferably being present on the cell surface of a target cell, preferably is externalized.

The antibody, which binds to human SRRM2, preferably is not an intracellular antibody.

The antibody, which binds to human SRRM2, preferably is
(a) an antibody comprising a heavy chain variable region having an amino acid sequence with at least 85% identity to the amino acid sequence shown in SEQ ID NO: 1 and a light chain variable region having an amino acid sequence with at least 85% identity to the amino acid sequence shown in SEQ ID NO: 2;
(b) an antibody comprising a heavy chain variable region having an amino acid sequence with at least 85% identity to the amino acid sequence shown in SEQ ID NO: 3 and a light chain variable region having an amino acid sequence with at least 85% identity to the amino acid sequence shown in SEQ ID NO: 4;
(c) an antibody comprising a heavy chain variable region having an amino acid sequence with at least 85% identity to the amino acid sequence shown in SEQ ID NO: 5 and a light chain variable region having an amino acid sequence with at least 85% identity to the amino acid sequence shown in SEQ ID NO: 6;
(d) an antibody comprising a heavy chain variable region having an amino acid sequence with at least 85% identity to the amino acid sequence shown in SEQ ID NO: 5 and a light chain variable region having an amino acid sequence with at least 85% identity to the amino acid sequence shown in SEQ ID NO: 26; or
(e) an antibody, which binds to the same epitope as that in the human SRRM2 protein to which any one of the antibodies (a) to (d) bind.

The antibody, which binds to human SRRM2, preferably is
(a) an antibody comprising a heavy chain variable region comprising heavy chain CDR1 having the amino acid sequence as set forth in SEQ ID NO: 7, heavy chain CDR2 having the amino acid sequence as set forth in SEQ ID NO: 8, and heavy chain CDR3 having the amino acid sequence as set forth in SEQ ID NO: 9, and
a light chain variable region comprising light chain CDR1 having the amino acid sequence as set forth in SEQ ID NO: 10, light chain CDR2 having the amino acid sequence as set forth in SEQ ID NO: 11, and light chain CDR3 having the amino acid sequence as set forth in SEQ ID NO: 12;
(b) an antibody comprising a heavy chain variable region comprising heavy chain CDR1 having the amino acid sequence as set forth in SEQ ID NO: 13, heavy chain CDR2 having the amino acid sequence as set forth in SEQ ID NO: 14, and heavy chain CDR3 having the amino acid sequence as set forth in SEQ ID NO: 15, and
a light chain variable region comprising light chain CDR1 having the amino acid sequence as set forth in SEQ ID NO: 16, light chain CDR2 having the amino acid sequence as set forth in SEQ ID NO: 17, and light chain CDR3 having the amino acid sequence as set forth in SEQ ID NO: 18;
(c) an antibody comprising a heavy chain variable region comprising heavy chain CDR1 having the amino acid sequence as set forth in SEQ ID NO: 19, heavy chain CDR2 having the amino acid sequence as set forth in SEQ ID NO: 20, and heavy chain CDR3 having the amino acid sequence as set forth in SEQ ID NO: 21, and
a light chain variable region comprising light chain CDR1 having the amino acid sequence as set forth in SEQ ID NO: 22, light chain CDR2 having the amino acid sequence as set forth in SEQ ID NO: 23, and light chain CDR3 having the amino acid sequence as set forth in SEQ ID NO: 24;
(d) an antibody comprising a heavy chain variable region comprising heavy chain CDR1 having the amino acid sequence as set forth in SEQ ID NO: 19, heavy chain CDR2 having the amino acid sequence as set forth in SEQ ID NO: 20, and heavy chain CDR3 having the amino acid sequence as set forth in SEQ ID NO: 21, and
a light chain variable region comprising light chain CDR1 having the amino acid sequence as set forth in SEQ ID NO: 27, light chain CDR2 having the amino acid sequence as set forth in SEQ ID NO: 28, and light chain CDR3 having the amino acid sequence as set forth in SEQ ID NO: 29; or
(e) an antibody, which binds to the same epitope as that in the human SRRM2 protein to which any one of the antibodies (a) to (d) bind.

The present invention further relates to the pharmaceutical composition of the invention for use in a method for the treatment of cancer in a human subject. Preferably, said cancer is characterized by cells, wherein human SRRM2 is present on their cell surface. Preferably, said cancer is breast cancer, prostate cancer, lung cancer, colorectal cancer, ovarian cancer, pancreatic cancer, stomach cancer, cholangiogenic cancer, oral cavity squamous carcinoma, hematologic malignancy tumor, or glioma.

The present invention further relates to a method for determining whether a human subject may suffer from cancer, comprising determining in a sample obtained from said human subject whether SRRM2 is present on the cell surface of cells comprised by said sample.

The present invention further relates to a method for determining whether a human subject may suffer from cancer, comprising determining in a sample obtained from said human subject whether SRRM2 is present on the surface of extracellular vesicles.

The present invention further relates to an antibody, which binds to human SRRM2 present on the cell surface of a target cell, wherein said antibody is
(a) an antibody comprising a heavy chain variable region having an amino acid sequence with at least 85% identity to the amino acid sequence shown in SEQ ID NO: 1 and a light chain variable region having an amino acid sequence with at least 85% identity to the amino acid sequence shown in SEQ ID NO: 2;
(b) an antibody comprising a heavy chain variable region having an amino acid sequence with at least 85% identity to the amino acid sequence shown in SEQ ID NO: 3 and a light chain variable region having an amino acid sequence with at least 85% identity to the amino acid sequence shown in SEQ ID NO: 4;
(c) an antibody comprising a heavy chain variable region having an amino acid sequence with at least 85% identity to the amino acid sequence shown in SEQ ID NO: 5 and a light chain variable region having an amino acid sequence with at least 85% identity to the amino acid sequence shown in SEQ ID NO: 6;
(d) an antibody comprising a heavy chain variable region having an amino acid sequence with at least 85% identity to the amino acid sequence shown in SEQ ID NO: 5 and a light chain variable region having an amino acid sequence with at least 85% identity to the amino acid sequence shown in SEQ ID NO: 26; or
(e) an antibody, which binds to the same epitope as that in the human SRRM2 protein to which any one of the antibodies (a) to (d) bind.

The present invention further relates to an antibody, which binds to human SRRM2 present on the cell surface of a target cell, comprising
(a) an antibody comprising a heavy chain variable region comprising heavy chain CDR1 having the amino acid sequence as set forth in SEQ ID NO: 7, heavy chain CDR2 having the amino acid sequence as set forth in SEQ ID NO: 8, and heavy chain CDR3 having the amino acid sequence as set forth in SEQ ID NO: 9, and
a light chain variable region comprising light chain CDR1 having the amino acid sequence as set forth in SEQ ID NO: 10, light chain CDR2 having the amino acid sequence as set forth in SEQ ID NO: 11, and light chain CDR3 having the amino acid sequence as set forth in SEQ ID NO: 12;
(b) an antibody comprising a heavy chain variable region comprising heavy chain CDR1 having the amino acid sequence as set forth in SEQ ID NO: 13, heavy chain CDR2 having the amino acid sequence as set forth in SEQ ID NO: 14, and heavy chain CDR3 having the amino acid sequence as set forth in SEQ ID NO: 15, and
a light chain variable region comprising light chain CDR1 having the amino acid sequence as set forth in SEQ ID NO: 16, light chain CDR2 having the amino acid sequence as set forth in SEQ ID NO: 17, and light chain CDR3 having the amino acid sequence as set forth in SEQ ID NO: 18;
(c) an antibody comprising a heavy chain variable region comprising heavy chain CDR1 having the amino acid sequence as set forth in SEQ ID NO: 19, heavy chain CDR2 having the amino acid sequence as set forth in SEQ ID NO: 20, and heavy chain CDR3 having the amino acid sequence as set forth in SEQ ID NO: 21, and
a light chain variable region comprising light chain CDR1 having the amino acid sequence as set forth in SEQ ID NO: 22, light chain CDR2 having the amino acid sequence as set forth in SEQ ID NO: 23, and light chain CDR3 having the amino acid sequence as set forth in SEQ ID NO: 24;
(d) an antibody comprising a heavy chain variable region comprising heavy chain CDR1 having the amino acid sequence as set forth in SEQ ID NO: 19, heavy chain CDR2 having the amino acid sequence as set forth in SEQ ID NO: 20, and heavy chain CDR3 having the amino acid sequence as set forth in SEQ ID NO: 21, and
a light chain variable region comprising light chain CDR1 having the amino acid sequence as set forth in SEQ ID NO: 27, light chain CDR2 having the amino acid sequence as set forth in SEQ ID NO: 28, and light chain CDR3 having the amino acid sequence as set forth in SEQ ID NO: 29; or
(e) an antibody, which binds to the same epitope as that in the human SRRM2 protein to which any one of the antibodies (a) to (d) bind.

The present invention further relates to an antibody, which binds to human Serine/arginine repetitive matrix protein 2 (SRRM2) present on the cell surface of a target cell for use in a method of killing said target cell having SRRM2 present on the cell surface.

Such an antibody preferably is
(a) an antibody comprising a heavy chain variable region having an amino acid sequence with at least 85% identity to the amino acid sequence shown in SEQ ID NO: 1 and a light chain variable region having an amino acid sequence with at least 85% identity to the amino acid sequence shown in SEQ ID NO: 2;
(b) an antibody comprising a heavy chain variable region having an amino acid sequence with at least 85% identity to the amino acid sequence shown in SEQ ID NO: 3 and a light chain variable region having an amino acid sequence with at least 85% identity to the amino acid sequence shown in SEQ ID NO: 4;
(c) an antibody comprising a heavy chain variable region having an amino acid sequence with at least 85% identity to the amino acid sequence shown in SEQ ID NO: 5 and a light chain variable region having an amino acid sequence with at least 85% identity to the amino acid sequence shown in SEQ ID NO: 6;
(d) an antibody comprising a heavy chain variable region having an amino acid sequence with at least 85% identity to the amino acid sequence shown in SEQ ID NO: 5 and a light chain variable region having an amino acid sequence with at least 85% identity to the amino acid sequence shown in SEQ ID NO: 26; or
(e) an antibody, which binds to the same epitope as that in the human SRRM2 protein to which any one of the antibodies (a) to (d) bind.

Such an antibody preferably is or comprises
(a) an antibody comprising a heavy chain variable region having an amino acid sequence with at least 85% identity to the amino acid sequence shown in SEQ ID NO: 1 and a light chain variable region having an amino acid sequence with at least 85% identity to the amino acid sequence shown in SEQ ID NO: 2;
(b) an antibody comprising a heavy chain variable region having an amino acid sequence with at least 85% identity to the amino acid sequence shown in SEQ ID NO: 3 and a light chain variable region having an amino acid sequence with at least 85% identity to the amino acid sequence shown in SEQ ID NO: 4;
(c) an antibody comprising a heavy chain variable region having an amino acid sequence with at least 85% identity to the amino acid sequence shown in SEQ ID NO: 5 and a light chain variable region having an amino acid sequence with at least 85% identity to the amino acid sequence shown in SEQ ID NO: 6;
(d) an antibody comprising a heavy chain variable region having an amino acid sequence with at least 85% identity to the amino acid sequence shown in SEQ ID NO: 5 and a light chain variable region having an amino acid sequence with at least 85% identity to the amino acid sequence shown in SEQ ID NO: 26; or
(e) an antibody, which binds to the same epitope as that in the human SRRM2 protein to which any one of the antibodies (a) to (d) bind.

The present invention further relates to a method of killing a target cell having Serine/arginine repetitive matrix protein 2 (SRRM2) present on the cell surface of said target cell, comprising administering an antibody, which binds to human SRRM2 present on the cell surface of said target cell.

For said method of killing, it is preferred that the antibody is
(a) an antibody comprising a heavy chain variable region having an amino acid sequence with at least 85% identity to the amino acid sequence shown in SEQ ID NO: 1 and a light chain variable region having an amino acid sequence with at least 85% identity to the amino acid sequence shown in SEQ ID NO: 2;
(b) an antibody comprising a heavy chain variable region having an amino acid sequence with at least 85% identity to the amino acid sequence shown in SEQ ID NO: 3 and a light chain variable region having an amino acid sequence with at least 85% identity to the amino acid sequence shown in SEQ ID NO: 4;
(c) an antibody comprising a heavy chain variable region having an amino acid sequence with at least 85% identity to the amino acid sequence shown in SEQ ID NO: 5 and a light chain variable region having an amino acid sequence with at least 85% identity to the amino acid sequence shown in SEQ ID NO: 6;
(d) an antibody comprising a heavy chain variable region having an amino acid sequence with at least 85% identity to the amino acid sequence shown in SEQ ID NO: 5 and a light chain variable region having an amino acid sequence with at least 85% identity to the amino acid sequence shown in SEQ ID NO: 26; or
(e) an antibody, which binds to the same epitope as that in the human SRRM2 protein to which any one of the antibodies (a) to (d) bind.

For said method of killing, the antibody preferably is
(a) an antibody comprising a heavy chain variable region comprising heavy chain CDR1 having the amino acid sequence as set forth in SEQ ID NO: 7, heavy chain CDR2 having the amino acid sequence as set forth in SEQ ID NO: 8, and heavy chain CDR3 having the amino acid sequence as set forth in SEQ ID NO: 9, and a light chain variable region comprising light chain CDR1 having the amino acid sequence as set forth in SEQ ID NO: 10, light chain CDR2 having the amino acid sequence as set forth in SEQ ID NO: 11, and light chain CDR3 having the amino acid sequence as set forth in SEQ ID NO: 12;
(b) an antibody comprising a heavy chain variable region comprising heavy chain CDR1 having the amino acid sequence as set forth in SEQ ID NO: 13, heavy chain CDR2 having the amino acid sequence as set forth in SEQ ID NO: 14, and heavy chain CDR3 having the amino acid sequence as set forth in SEQ ID NO: 15, and a light chain variable region comprising light chain CDR1 having the amino acid sequence as set forth in SEQ ID NO: 16, light chain CDR2 having the amino acid sequence as set forth in SEQ ID NO: 17, and light chain CDR3 having the amino acid sequence as set forth in SEQ ID NO: 18;
(c) an antibody comprising a heavy chain variable region comprising heavy chain CDR1 having the amino acid sequence as set forth in SEQ ID NO: 19, heavy chain CDR2 having the amino acid sequence as set forth in SEQ ID NO: 20, and heavy chain CDR3 having the amino acid sequence as set forth in SEQ ID NO: 21, and a light chain variable region comprising light chain CDR1 having the amino acid sequence as set forth in SEQ ID NO: 22, light chain CDR2 having the amino acid sequence as set forth in SEQ ID NO: 23, and light chain CDR3 having the amino acid sequence as set forth in SEQ ID NO: 24;
(d) an antibody comprising a heavy chain variable region comprising heavy chain CDR1 having the amino acid sequence as set forth in SEQ ID NO: 19, heavy chain CDR2 having the amino acid sequence as set forth in SEQ ID NO: 20, and heavy chain CDR3 having the amino acid sequence as set forth in SEQ ID NO: 21, and a light chain variable region comprising light chain CDR1 having the amino acid sequence as set forth in SEQ ID NO: 27, light chain CDR2 having the amino acid sequence as set forth in SEQ ID NO: 28, and light chain CDR3 having the amino acid sequence as set forth in SEQ ID NO: 29; or
(e) an antibody, which binds to the same epitope as that in the human SRRM2 protein to which any one of the antibodies (a) to (d) bind.

The invention further relates to an antibody, which binds to human Serine/arginine repetitive matrix protein 2 (SRRM2) present on the cell surface of a target cell for use in a method for the treatment of cancer in a subject, comprising, prior to the treatment of cancer in said subject, determining whether a target cell of said subject has SRRM2 present on the cell surface.

Such an antibody, which binds to human SRRM2 present on the cell surface of a target cell for use in a method for the treatment of cancer in a subject, comprising, prior to the treatment of cancer in said subject, determining whether a target cell of said subject has SRRM2 present on the cell surface, preferably is
(a) an antibody comprising a heavy chain variable region having an amino acid sequence with at least 85% identity to the amino acid sequence shown in SEQ ID NO: 1 and a light chain variable region having an amino acid sequence with at least 85% identity to the amino acid sequence shown in SEQ ID NO: 2;
(b) an antibody comprising a heavy chain variable region having an amino acid sequence with at least 85% identity to the amino acid sequence shown in SEQ ID NO: 3 and a light chain variable region having an amino acid sequence with at least 85% identity to the amino acid sequence shown in SEQ ID NO: 4;
(c) an antibody comprising a heavy chain variable region having an amino acid sequence with at least 85% identity to the amino acid sequence shown in SEQ ID NO: 5 and a light chain variable region having an amino acid sequence with at least 85% identity to the amino acid sequence shown in SEQ ID NO: 6;
(d) an antibody comprising a heavy chain variable region having an amino acid sequence with at least 85% identity to the amino acid sequence shown in SEQ ID NO: 5 and a light chain variable region having an amino acid sequence with at least 85% identity to the amino acid sequence shown in SEQ ID NO: 26; or
(e) an antibody, which binds to the same epitope as that in the human SRRM2 protein to which any one of the antibodies (a) to (d) bind.

Such an antibody, which binds to human SRRM2 present on the cell surface of a target cell for use in a method for the treatment of cancer in a subject, comprising, prior to the treatment of cancer in said subject, determining whether a target cell of said subject has SRRM2 present on the cell surface, preferably is or comprises
(a) an antibody comprising a heavy chain variable region comprising heavy chain CDR1 having the amino acid sequence as set forth in SEQ ID NO: 7, heavy chain CDR2 having the amino acid sequence as set forth in SEQ ID NO: 8, and heavy chain CDR3 having the amino acid sequence as set forth in SEQ ID NO: 9, and a light chain variable region comprising light chain CDR1 having the amino acid sequence as set forth in SEQ ID NO: 10, light chain CDR2 having the amino acid sequence as set forth in SEQ ID NO: 11, and light chain CDR3 having the amino acid sequence as set forth in SEQ ID NO: 12;
(b) an antibody comprising a heavy chain variable region comprising heavy chain CDR1 having the amino acid sequence as set forth in SEQ ID NO: 13, heavy chain CDR2 having the amino acid sequence as set forth in SEQ ID NO: 14, and heavy chain CDR3 having the amino acid sequence as set forth in SEQ ID NO: 15, and a light chain variable region comprising light chain CDR1 having the amino acid sequence as set forth in SEQ ID NO: 16, light chain CDR2 having the amino acid sequence as set forth in SEQ ID NO: 17, and light chain CDR3 having the amino acid sequence as set forth in SEQ ID NO: 18;
(c) an antibody comprising a heavy chain variable region comprising heavy chain CDR1 having the amino acid sequence as set forth in SEQ ID NO: 19, heavy chain CDR2 having the amino acid sequence as set forth in SEQ ID NO: 20, and heavy chain CDR3 having the amino acid sequence as set forth in SEQ ID NO: 21, and a light chain variable region comprising light chain CDR1 having the amino acid sequence as set forth in SEQ ID NO: 22, light chain CDR2 having the amino acid sequence as set forth in SEQ ID NO: 23, and light chain CDR3 having the amino acid sequence as set forth in SEQ ID NO: 24;
(d) an antibody comprising a heavy chain variable region comprising heavy chain CDR1 having the amino acid sequence as set forth in SEQ ID NO: 19, heavy chain CDR2 having the amino acid sequence as set forth in SEQ ID NO: 20, and heavy chain CDR3 having the amino acid sequence as set forth in SEQ ID NO: 21, and a light chain variable region comprising light chain CDR1 having the amino acid sequence as set forth in SEQ ID NO: 27, light chain CDR2 having the amino acid sequence as set forth in SEQ ID NO: 28, and light chain CDR3 having the amino acid sequence as set forth in SEQ ID NO: 29; or
(e) an antibody, which binds to the same epitope as that in the human SRRM2 protein to which any one of the antibodies (a) to (d) bind.

### BRIEF DESCRIPTION OF THE DRAWINGS

FIG. 1 shows that the antibodies 13F11, 23A7 and 18A4 precipitate SRRM2.

Cyanogen bromide beads coupled with any of the three antibodies or with an isotype control antibody were incubated overnight with lysates from OVCAR-3 or UWB1.289 ovarian cancer cells. After precipitation of the beads and incubation in Laemmli buffer, eluates were analyzed by mass spectrometry, which revealed a clear enrichment of SRRM2 with either antibody as compared to the isotype control antibody.

FIG. 2 shows the surface expression of SRRM2 on various cancer cell lines. Cell lines were incubated with 13F11 (solid lines in FIG. 2A and FIG. 2B) or an isotype control antibody (dotted line in FIG. 2A, tinted histogram in FIG. 2B). Cells were then washed and incubated with an Alexa647 labeled anti-rat IgG antibody. Fluorescence was then measured with a FACS Canto. Normal PBMCs do not express surface SRRM2 (not shown).

FIG. 3 shows that 13F11, 23A7 and 18A4 are SRRM2-specific: A549 cells were transfected with a ribonucleoprotein complex consisting of SRRM2-specific gRNAs and Cas9. Seven days later, binding of the antibodies to knockout and wildtype cells was measured by flow cytometry. For this, A549 Wildtype (bold line) and the A549 SRRM Knockout line #25 (tinted histogram) were incubated with any of the three antibodies 13F11, 18A4 and 23A7. Cells were washed and then incubated with an Alexa647 labeled anti-rat secondary antibody. Fluorescence was then measured with a FACS Canto. An isotype control antibody was included as a negative control (dotted line).

FIG. 4 shows that 13F11 binds to vital SK-OV-3 cells. SK-OV-3 cells were grown on microscope slides, washed in PBS, and incubated with the antibody 13F11. Then, cells were fixed with 4% paraformaldehyde and incubated with an Alexa647-coupled suitable anti-rat IgG secondary antibody. Surface staining is clearly visible.

FIG. 5 shows that 18A4 co-localizes with the surface molecule CD47. A549 or UWB1.289 cells were cultivated on a cover slip, washed and incubated with the antibodies 18A4 (SRRM2, magenta) and 18F10 (CD47, green). Cells were fixed with 4% paraformaldehyde and analyzed by confocal fluorescence microscopy. Nuclei were counter-stained with DAPI (blue).

FIG. 6 shows that SRRM2 localizes to lipid rafts or another kind of insoluble membrane subdomains. Cells were lysed in RIPA buffer and the lysate was put onto of a density gradient. After centrifugation, 10 fractions of 400 μl each were taken and samples of these fractions were spotted onto a nitrocellulose membrane. The membrane was incubated with antibody 13F11 or with an antibody specific for the tetraspanin CD63. Fractions 2 and 3 correspond to a density of approximately 1.13-1.19 g/ml, and thus in the range of lipid rafts. Soluble proteins, which have higher specific density, are mainly located in fractions 7-10.

FIG. 7 shows that surface-SRRM2-positive SK-OV-3-cells activate SRRM2-specific 13F11 CAR-T-cells. Primary T-cells from two donors (1 and 2) were transduced with a retroviral vector encoding a CAR construct with a single-chain construct derived from antibody 13F11. Expression of the CAR was tested by flow cytometry with a goat-anti rat IgG antibody (not shown). SK-OV-3 cells were incubated with 13F11 CAR-T cells (columns 3-4 of the blots) of non-transduced control T cells (column 1-2 of the blots) at the ratios indicated on the X-axis for 24 h. Then, interferon-gamma in the supernatants, as a marker for T-cell activation, was quantified with a commercial ELISA assay.

FIG. 8 shows that SRRM2-specific CAR-T cells kill SRRM2-positive HO-8910 target cells. Primary T-cells from one donors were transduced with a retroviral vector encoding a CAR construct with a single-chain construct derived from antibody 13F11. Expression of the CAR was tested by flow cytometry with a goat-anti rat IgG antibody (not shown). SK-OV-3 cells were incubated with 13F11 CAR-T cells of non-transduced control T cells (Mock-T) at the ratios indicated on the X-axis for 24 h. Then, killing was measured with a commercial lactate dehydrogenase (LDH) assay.

FIG. 9 shows that SRRM2 is released via extracellular vesicles (EVs): EVs isolated from ascites of a patient with ovarian cancer were isolated by established technologies comprising differential centrifugation, ultracentrifugation, and density gradient purification. 2 μl each of this EV preparation were then spotted onto pieces of nitrocellulose membrane and incubated (from left to right) with 13F11, 18A4, 23A7 or an isotype control antibody at 4° C. overnight. After washing, membranes were incubated with a suitable secondary antibody (goat anti-rat IgG, coupled with horseradish peroxidase) for 2 h and developed with ECL.

FIG. 10 shows that SRRM2 surface expression on cancer cells is induced by hypoxia. Various cancer cell lines were incubated in normoxia or hypoxia (1% O₂) for 72 hours. SRRM2 surface expression was measured with 13F11 labeled with Alexa647. Mean fluorescence levels are given. An isotype antibody was used as a control.

FIG. 11 shows that the antibody 13F11 is internalized upon binding to SRRM2. SK-OV-3 ovarian cancer cells were incubated with 13F11 for 10 min at room temperature and then washed to remove free antibody. Cell samples were then incubated at 37° C. for one to five hours or kept on ice (=0 hour). Cells were then stained with an Alexa647-coupled secondary anti-rat IgG antibody and fluorescence was measured by flow cytometry. The decrease in fluorescence over time is indicative for the internalization of the SRRM2-13F11 complex. The fluorescence measured after 0 hours at 37° C. was set to 100%.

FIG. 12 shows sections of a pancreatic cancer and normal pancreas, stained with 18A4. Membrane staining is visible in cancer cells, while normal pancreas cell reveal nuclear staining.

FIG. 13 shows membrane localization of SRRM2 in xenotransplanted human HUCCT-1 cells. Again, normal mouse tissue clearly reveal nuclear staining, only.

FIG. 14 shows the respective sequences of the antibodies 13F11, 23A7 and 18A4, including the light chains and heavy chains with the respective CDR1, CDR2 and CDR3 sequences.

FIG. 15 shows that 13F11, 18A4-2 and 23A7 binds to a fragment (called herein tr04, SEQ ID NO: 30) spanning amino acids 1889 to 2150 of SRRM2. HEK293 cells were transfected with an expression plasmid encoding amino acids 1889 to 2150 of SRRM2 fused to a HIS-tag (HEK293-tr04). Immunoprecipitations were performed with bead-coupled SRRM2-specific antibodies 13F11, 18A4-2 and 23A7 and lysates of transfected cells or, as control, non-transfected 293 cells (HEK293). Precipitated proteins were separated by PAGE and transferred to a PVDF membrane, which was then incubated with an anti-rat antibody coupled to HRP. Finally, the blot was developed with ECL. As additional controls, lysates from transfected and non-transfected cells were included.

FIG. 16 shows that normal tissues from Cynomolgus monkeys either stain negative for SRRM2 or show cytoplasmic/nuclear staining as revealed by immunohistochemistry. No membrane staining was observed. Mem=membrane; Cyto=cytoplasmic; Nucl=nuclear. FFPE tissues from cynomolgus monkey were incubated with 13F11 antibody, followed by incubation with a goat anti-rat IgG antibody coupled with HRP (ImmPRESS HRP Peroxidase detection kit; https://vectorlabs.com/products/enzyme-polymer/immpress-hrp-goat-anti-rat-igg-kit).

FIG. 17 shows that the majority of tumor samples derived from different locations (bile duct, ovary, pancreas, stomach) reveal SRRM2 membrane staining. Formalin-fixed paraffin embedded (FFPE) tumor tissues and adjacent normal tissues were stained with antibody 13F11, followed by incubation with a rat-specific secondary antibody coupled with HRP. Tissues were inspected by a trained pathologist and staining intensity was scored from 0 (negative) to 3+ (strong expression on the majority of cells).

FIG. 18 shows the sequences of the variable parts of the light and heavy chains of antibody 18A4-2.

FIG. 19 shows that the SRRM2-specific antibodies 13F11, 18A4-2 and 23A7 bind to fragment tr04. A 96-well cell cluster plate was coated with purified SRRM2-tr04-HIS or, as a control, MISP-HIS protein (each at 50 μg/ml) overnight and then blocked with non-fat milk powder in TBST. Then, antibodies 13F11, 18A4-2, 23A7 or an anti-MISP antibody were added for 2 hours at room temperature. After washing, the plate was incubated with a secondary anti-rat IgG antibody coupled to HRP and developed with TMB. After stopping the reaction with H₂SO₄, the absorbance was measured at 450 nm.

### DETAILED DESCRIPTION OF THE INVENTION

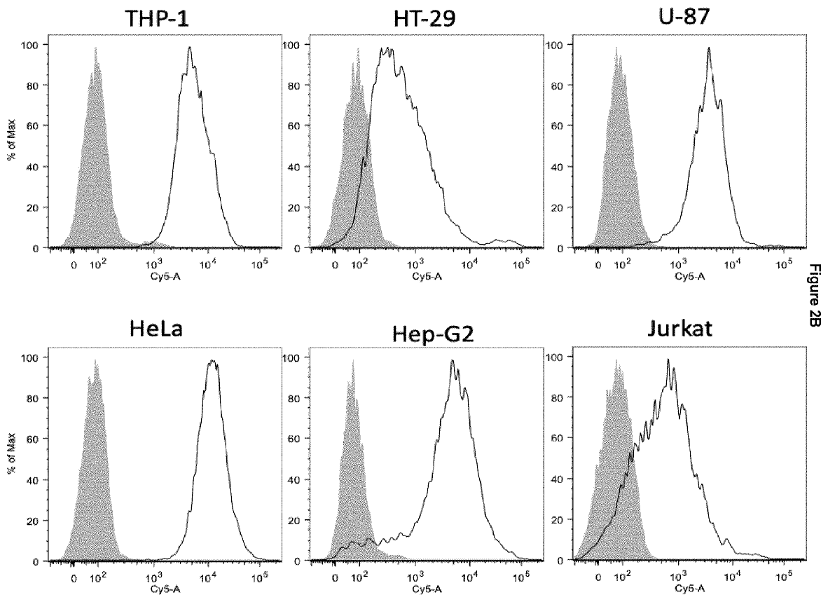

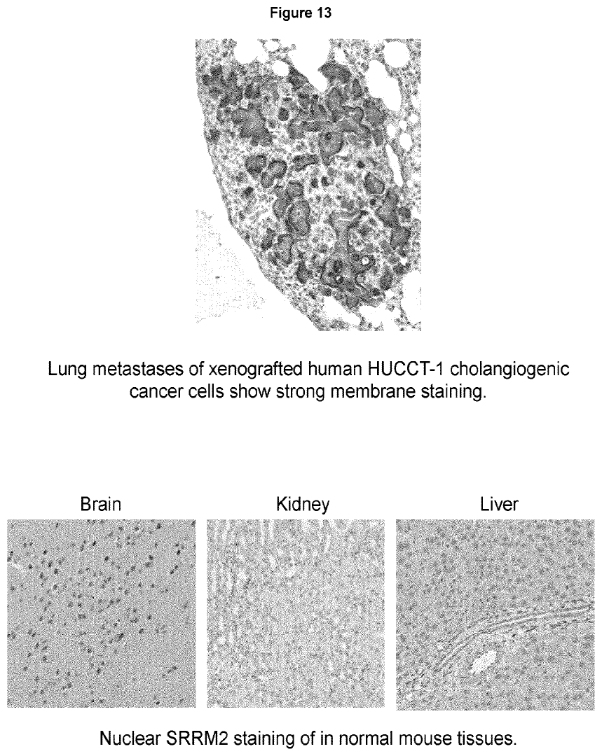

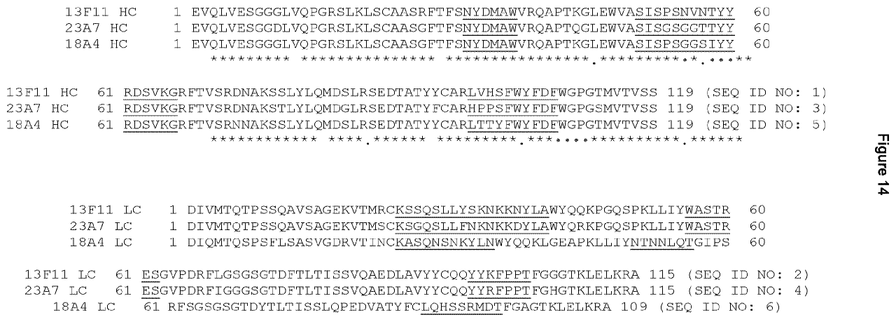

The present inventors surprisingly found that SRRM2 is present on the cell surface of cancer cells (see e.g., FIGS. 2-5, 12 and 13) although widely described in prior art as being a protein localized in the nucleus, in particular in nuclear speckles. Serine/arginine repetitive matrix protein 2 (SRRM2; UniProt Q9UQ35) plays an important role in pre-mRNA splicing and is a major component of the spliceosome. As such, it is not present on the cell surface of healthy cells. However, within the context of cancer, SRRM can be seen as becoming “externalized”, i.e. it is no longer not only present in the nucleus, but also present on the cell surface of a then cancer cell. Thus, up to now, SRRM2 was not known to be available for therapy for being localized in the nucleus, in particular in the nuclear speckles. However, as the present inventors surprisingly found that SRRM2 is present on the cell surface of cancer cells, those cells can now be targeted for the treatment of cancer. An exemplary sequence of SRRM2 is provided by UniProt database entry Q9UQ35, version 2 of 6 Mar. 2007 and exemplified in SEQ ID NO: 25.

The SRRM2 protein within the context of the present invention may be a SRRM2 protein having SEQ ID NO: 25 or may be a modified protein having an amino acid sequence derived from the sequence described above by the modification of one or more amino acids. Examples of the modified protein having a sequence derived from the sequence described above by the modification of one or more amino acids can include polypeptides having 70% or more, preferably 80% or more, more preferably 90% or more, even more preferably 95% or more homology to the amino acid sequence. Alternatively, partial peptides of these SRRM2 proteins may be used.

The term “homology” as used herein in its usual meaning includes identical amino acids as well as amino acids, which are regarded to be conservative substitutions (for example, exchange of a glutamate residue by an aspartate residue) at equivalent positions in the linear amino acid sequence of two proteins that are compared with each other. By “identity” or “sequence identity” is meant a property of sequences that measures their similarity or relationship. The term “sequence identity” or “identity” as used in the present invention means the percentage of pair-wise identical residues—following (homology) alignment of a sequence of a polypeptide of the invention with a sequence in question—with respect to the number of residues in the longer of these two sequences. Identity is measured by dividing the number of identical residues by the total number of residues and multiplying the product by 100.

The percentage of sequence homology or sequence identity can, for example, be determined herein using the program BLASTP, version blastp 2.2.5 (Nov. 16, 2002; cf. Altschul, S. F. et al. (1997) Nucl. Acids Res. 25, 3389-3402). In this embodiment, the percentage of homology is based on the alignment of the entire polypeptide sequences (matrix: BLOSUM 62; gap costs: 11.1; cutoff value set to 10-3), optionally including the propeptide sequences, using the human IL-4 as reference in a pairwise comparison. It is calculated as the percentage of numbers of “positives” (homologous amino acids) indicated as result in the BLASTP program output divided by the total number of amino acids selected by the program for the alignment. It is noted in this connection that this total number of selected amino acids can differ from the length of the porcine NTCP.

The SRRM2 protein used in the present invention is not limited by its origin and is preferably a human SRRM2 protein.

The term “antibody” as used herein and in the context of the present invention may comprise chimeric antibodies, humanized antibodies, monovalent antibodies, polyvalent antibodies, low-molecular antibodies, a diabody or a scFv.

Chimeric antibodies refer to antibodies comprising variable and constant regions of different origins ligated with each other. For example, mouse-human heterogeneous chimeric antibodies are antibodies comprising the heavy and light chain variable regions of a mouse antibody and the heavy and light chain constant regions of a human antibody. Mouse antibody variable region-encoding DNAs are ligated with human antibody constant region-encoding DNAs, and the ligation products can be incorporated into expression vectors to prepare chimeric antibody-expressing recombinant vectors. Cells transformed with these vectors (recombinant cells) can be cultured for the expression of the DNA insert to obtain the chimeric antibodies produced during the culture.

In general, the chimeric antibodies comprise non-human animal-derived antibody variable regions and human antibody-derived constant regions. By contrast, the humanized antibodies comprise non-human animal-derived antibody complementarity-determining regions (CDRs), human antibody-derived framework regions (FRs), and human antibody-derived constant regions. The humanized antibodies are also called reshaped human antibodies. Specifically, for example, humanized antibodies comprising non-human animal (e.g., mouse) antibody CDRs grafted in human antibodies are known in the art. The humanized antibodies are useful as active ingredients for a therapeutic agent of the present invention, owing to their reduced antigenicity in the human body.

Each antibody variable region usually comprises 3 CDRs flanked by 4 FRs. The CDR regions substantially determine the binding specificity of the antibody. The CDRs have diverse amino acid sequences. On the other hand, amino acid sequences constituting the FRs often exhibit high homology among antibodies having different binding specificities. Therefore, in general, the binding specificity of a certain antibody can allegedly be transplanted to other antibodies through CDR grafting.

The antibody, which binds to human SRRM2 present in a pharmaceutical composition of the present invention may encompass bivalent antibodies typified by IgG (IgG1, IgG2, IgG4, etc.) and also monovalent antibodies or polyvalent antibodies typified by IgM as long as these antibodies bind to the SRRM2 protein. The polyvalent antibody that may be present in the pharmaceutical composition of the present invention may encompass polyvalent antibodies having antigen-binding sites, all of which are the same as each other or some or all of which are different from each other. Preferably, the antibody, which binds to human SRRM2 is an IgG antibody.

The antibody, which binds to human SRRM2 present in the pharmaceutical composition of the present invention may also be a low-molecular antibody that encompasses an antibody fragment deficient in a portion of the whole antibody (e.g., whole IgG). Such partial deficiency of the antibody molecule is accepted as long as the resultant antibody fragment is capable of binding to the SRRM2. It is preferred that the antibody fragment employed in the pharmaceutical composition according to the present invention should contain one or both of heavy chain variable (VH) and light chain variable (VL) regions. It is also preferred that the antibody fragment employed in the pharmaceutical composition according to the present invention should contain CDRs. The number of CDRs contained in the antibody fragment employed in the pharmaceutical composition of the present invention is not particularly limited and is preferably at least 6 CDRs: heavy chain CDR1, CDR2, and CDR3 and light chain CDR1, CDR2, and CDR3.

The amino acid sequence of VH or VL can contain one or more substitution, deletion, addition, and/or insertion. Furthermore, the antibody or antibody fragment that may be employed in the pharmaceutical composition of the present invention may be deficient in a portion of one or both of VH and VL, as long as the resultant antibody fragment is capable of binding to the human SRRM2. Moreover, its variable region may be chimerized or humanized. Specific examples of the antibody fragment can include Fab, Fab′, F(ab′)2, and Fv. Moreover, specific examples of the low-molecular antibody can include Fab, Fab′, F(ab′)2, Fv, scFv (single chain Fv), diabody, sc(Fv)2 (single chain (Fv)2), and scFv-Fc. In the present invention, the low-molecular antibody is preferably a diabody or sc(Fv)2. These antibody multimers (e.g., dimers, trimers, tetramers, and polymers) are also encompassed by the low-molecular antibody that may be employed in the pharmaceutical composition of the present invention.

The term “diabody” as used herein and in the context of the present invention may refer to a bivalent antibody fragment constructed by gene fusion. The diabody is a dimer comprising two polypeptide chains. Usually, each of the polypeptide chains constituting the dimer comprises heavy and light chain variable regions linked via a linker on the same chain. The linker in the diabody is generally too short to allow paring between heavy and light chain variable regions on the same chain. Specifically, the number of amino acid residues constituting the linker is, for example, approximately 5 residues. Therefore, heavy and light chain variable regions encoded on the same polypeptide chain cannot together form a single chain variable region fragment. Instead, they form a dimer by pairing with another single chain variable region fragment. As a result, the diabody has two antigen-binding sites.

The scFv, as used in the context of the present invention, may be obtained by linking heavy and light chain variable regions of the antibody. In the scFv, the heavy and light chain variable regions are linked via a linker, preferably, a peptide linker. The heavy and light chain variable regions in the scFv can be derived from any of the antibodies described in the present specification. The peptide linker that links the variable regions is not particularly limited. For example, an arbitrary single chain peptide of approximately 3 to 25 residues can be used as the linker.

The antibody, which binds to human SRRM2 as used in the pharmaceutical composition and the antibody, which binds to human SRRM2 according to the present invention may also encompass binding entities, such as lipocalins, an aptamer or an anticalin.

The antibody, which binds to human SRRM2, used in the present invention can have various formats. However, it needs to bind to the SRRM2 protein and is not particularly limited by its origin, type, shape, etc., but may have a cytotoxic activity. Specifically, an antibody can be used, such as a non-human animal-derived antibody (e.g., a mouse, rat, or camel antibody), a human-derived antibody, a chimeric antibody, or a humanized antibody as described above. The antibody, which binds to human SRRM2 used in the present invention may in one embodiment be a polyclonal or monoclonal antibody and is preferably a monoclonal antibody.

The antibody, which binds to human SRRM2 used in the present invention can be obtained as a polyclonal or monoclonal antibody using means known in the art. The antibody used in the present invention is in particular preferably a mammal-derived monoclonal antibody. The mammal-derived monoclonal antibody encompasses, for example, those produced by hybridomas and those produced by hosts transformed with expression vectors containing an antibody gene by a genetic engineering approach.

The antibody, which binds to human SRRM2 may be modified with various molecules such as polyethylene glycol (PEG). Further, the antibody, which binds to human SRRM2 may also be modified with cytotoxic substance such as a chemotherapeutic agent, a radioactive chemical, or the like.

Specific examples of the antibody used in the present invention, which recognizes SRRM2 present on the cell surface of a target cell such as a cancer cell and binds thereto, may include the antibodies given and described herein.

As described above, an antibody, which binds to human SRRM2 of the present invention or as used in the pharmaceutical composition of the present invention or as used in the methods of the present invention including the substitution, deletion, addition, and/or insertion of one or more amino acids is also incorporated in the scope of the present invention and may be prepared or occur naturally. Examples of a method for introducing a mutation in the polypeptide include site-directed mutagenesis (Hashimoto-Gotoh, T. et al., 1995) (Zoller, M J, and Smith, M., 1983) (Kramer, W. et al., 1984) (Kramer W, and Fritz H J, 1987) (Kunkel, T A, 1985) (Kunkel, 1988). This is one of the methods well known by those skilled in the art for preparing a polypeptide functionally equivalent to a certain polypeptide. Those skilled in the art can appropriately introduce a mutation in the antibody of the present invention or the antibody as used in the pharmaceutical composition of the present invention or as used in the methods of the present invention using such a method and thereby prepare an antibody functionally equivalent to such antibody. Moreover, amino acid mutations may occur in the natural world. Such an antibody that has an amino acid sequence derived from the amino acid sequence of the antibody of the present invention or the antibody as used in the pharmaceutical composition of the present invention or as used in the methods of the present invention comprising the mutation of one or more amino acids, is functionally equivalent or a variant to the antibody and is also encompassed by the antibody of the present invention or the antibody as used in the pharmaceutical composition of the present invention or as used in the methods of the present invention.

The number of amino acids mutated in such a variant is usually within 50 amino acids, preferably within 30 amino acids, more preferably within 10 amino acids (e.g., within 5 amino acids).

For amino acid residues to be mutated, it is preferred that this mutation should be performed conservatively between amino acids having the same side chain property. For example, the following classification based on the properties of amino acid side chains has been established: hydrophobic amino acids (A, I, L, M, F, P, W, Y, and V), hydrophilic amino acids (R, D, N, C, E, Q, G, H, K, S, and T), amino acids having an aliphatic side chain (G, A, V, L, I, and P), amino acids having a hydroxyl group-containing side chain (S, T, and Y), amino acids having a sulphur atom-containing side chain (C and M), amino acids having a side chain containing carboxylic acid and amide (D, N, E, and Q), amino acids having a base-containing side chain (R, K, and H), and amino acids having an aromatic group-containing side chain (H, F, Y, and W) (all symbols within the parentheses represent single letter codes of amino acids).

A polypeptide having an amino acid sequence modified from a certain amino acid sequence by the deletion and/or addition of one or more amino acid residue(s) and/or the substitution thereof by other amino acids is already known to maintain the biological activity of the original polypeptide (Mark, D. F. et al., 1984) (Wang, A. et al., 1982). Specifically, when amino acids in an amino acid sequence constituting a certain polypeptide are substituted by amino acids classified in the same group thereas, it is generally said that the polypeptide is likely to maintain its activity. In the present invention, the substitution between amino acids within the same amino acid group described above is referred to as conservative substitution.

The term “surface” or specifically “cell surface” as used herein means the cell membrane. The cell membrane, which may also be known as plasma membrane, is the thin membrane that surrounds every living cell, delimiting the cell from the environment around it. Enclosed by this cell membrane are the cell's constituents, often large, water-soluble, highly charged molecules such as proteins, nucleic acids, carbohydrates, and substances involved in cellular metabolism. The cell membrane, therefore, has at least two functions: first, to be a barrier keeping the constituents of the cell in and unwanted substances out and, second, to be a gate allowing transport into the cell of essential nutrients and removal of waste products from the cell. ER-derived vesicles may also participate in the building or formation of the cell membrane.

The antibody, which binds to human SRRM2 preferably is capable of binding to a SRRM2 polypeptide extracellularly. In other words, an antibody as described herein may be capable of binding to SRRM2 when it is outside the cell, which also comprises SRRM2 to be specifically on the surface of the cell. Accordingly, the antibody, which binds to human SRRM2 preferably is no intracellular antibody, which is also called an intrabody. An “intrabody” (from intracellular and antibody) is an antibody that works within the cell to bind to an intracellular protein. This requires the expression of the antibody within the target cell, which can be accomplished, e.g., in transgenic animals or by gene therapy. As a result, intrabodies are antibodies that have been modified for intracellular localization and include antibodies, which are produced in prokaryotes or other non-target cells. The term “intrabody” can apply to several types of protein targeting: the antibody may remain in the cytoplasm, or it may have a nuclear localization signal, or it may undergo cotranslational translocation across the membrane into the lumen of the endoplasmic reticulum, provided that it is retained in that compartment through a KDEL sequence. However, the antibodies, which bind to human SRRM2 of the invention preferably are capable of being internalized as shown in Example 11.

The “target cell” preferably is a cancer cell. The term “cancer”, as used herein, can comprise any one or more of the following: acute lymphocytic leukemia (ALL), acute myeloid leukemia (AML), adrenocortical cancer, anal cancer, bladder cancer, blood cancer, bone cancer, brain tumor, breast cancer, cancer of the female genital system, cancer of the male genital system, central nervous system lymphoma, cervical cancer, childhood rhabdomyosarcoma, childhood sarcoma, chronic lymphocytic leukemia (CLL), chronic myeloid leukemia (CML), colon and rectal cancer, colon cancer, endometrial cancer, endometrial sarcoma, esophageal cancer, eye cancer, gallbladder cancer, gastric cancer, gastrointestinal tract cancer, hairy cell leukemia, head and neck cancer, hepatocellular cancer, Hodgkin's disease, hypopharyngeal cancer, Kaposi's sarcoma, kidney cancer, laryngeal cancer, leukemia, liver cancer, lung cancer, malignant fibrous histiocytoma, malignant thymoma, melanoma, mesothelioma, multiple myeloma, myeloma, nasal cavity and paranasal sinus cancer, nasopharyngeal cancer, nervous system cancer, neuroblastoma, non-Hodgkin's lymphoma, oral cavity cancer, oropharyngeal cancer, osteosarcoma, ovarian cancer, pancreatic cancer, parathyroid cancer, penile cancer, pharyngeal cancer, pituitary tumor, plasma cell neoplasm, primary CNS lymphoma, prostate cancer, rectal cancer, respiratory system cancer, retinoblastoma, salivary gland cancer, skin cancer, small intestine cancer, soft tissue sarcoma, stomach cancer, testicular cancer, thyroid cancer, urinary system cancer, uterine sarcoma, vaginal cancer, vascular system cancer, Waldenstrom's macroglobulinemia and Wilms' tumor.

The term “pharmaceutically acceptable carrier, diluent or excipient” as used in the context of the present invention, may comprise any pharmaceutically acceptable carrier, diluent or excipient for a pharmaceutical composition known by the person skilled in the art. It will be understood that such antibodies or pharmaceutical composition as described herein may be mixed with carriers or diluents, which will not interfere with the intended purpose of the present invention. For example, such a carrier as used within the present invention may be a carrier protein, such as bovine serum albumin (BSA) or keyhole limpet haemocyanin (KLH).

In one embodiment of the pharmaceutical composition of the present invention, said antibody, which binds to human SRRM2, has cytotoxic activity. In the context of the present invention, the phrase “cytotoxic activity” may refer to having a SRRM2 binding activity and may also include having an activity equivalent to that of the antibody, which binds to human SRRM2 of the present invention. In the present invention, the equivalent activity is not necessarily required to be an identical activity and may be, for example, 50% or more, preferably 70% or more, more preferably 90% or more activity compared with the activity of any of the antibodies (a) to (e) as described herein. Examples of the upper limit of the activity can include, but is not particularly limited to, 1000% or less, 500% or less, 300% or less, 150% or less, and 100% or less.

In one further embodiment of the pharmaceutical composition of the present invention, said antibody, which binds to human SRRM2 has (antibody-dependent cell-mediated cytotoxicity) ADCC or complement-dependent cytotoxicity (CDC). Thus, examples of the cytotoxic activity according to the present invention can include ADCC and/or CDC activities. In the context of the present invention, the ADCC activity means the activity of damaging target cells through the binding of Fcγ receptor-bearing cells (immunocytes, etc.) via the Fcγ receptors to the Fc domains of antibodies specifically attached to the cell surface antigens of the target cells. On the other hand, the CDC activity means a cytotoxic activity mediated by the complement system. Whether or not the antibody has an ADCC activity or has a CDC activity can be determined by a method known in the art.

Thus, the antibody, which binds to human SRRM2 employed in the pharmaceutical composition of the present invention may have activities such as an ADCC activity and as such, may be useful as a pharmaceutical drug, preferably, an anti-cancer agent, wherein the cancer is e.g. breast cancer, prostate cancer, lung cancer, colorectal cancer, ovarian cancer, pancreatic cancer, stomach cancer, cholangiogenic cancer, oral cavity squamous carcinoma, hematologic malignancy tumor, or glioma.

In one embodiment of the present invention, said antibody, which binds to human SRRM2, is conjugated with a cytotoxic substance.

In one preferred embodiment, the antibody, which binds to human SRRM2 may be conjugated with a cytotoxic substance, such as a chemotherapeutic agent, a toxic peptide, or a radioactive chemical. Such a modified antibody (hereinafter, referred to as an antibody conjugate) can be obtained by chemically modifying the obtained antibody. A method for the antibody modification has already been established in the art.

Examples of the chemotherapeutic agent whose cytotoxic activity functions through the conjugation to the antibody that binds SRRM2 can include the following chemotherapeutic agents: azaribine, anastrozole, azacytidine, bleomycin, bortezomib, bryostatin-1, busulfan, camptothecin, 10-hydroxycamptothecin, carmustine, celebrex, chlorambucil, cisplatin, irinotecan, carboplatin, cladribine, cyclophosphamide, cytarabine, dacarbazine, docetaxel, dactinomycin, daunomycin glucuronide, daunorubicin, dexamethasone, diethylstilbestrol, doxorubicin, doxorubicin glucuronide, epirubicin, ethinyl estradiol, estramustine, etoposide, etoposide glucuronide, floxuridine, fludarabine, flutamide, fluorouracil, fluoxymesterone, gemcitabine, hydroxyprogesterone caproate, hydroxyurea, idarubicin, ifosfamide, leucovorin, lomustine, mechlorethamine, medroxyprogesterone acetate, megestrol acetate, melphalan, mercaptopurine, methotrexate, mitoxantrone, mithramycin, mitomycin, mitotane, phenylbutyrate, prednisone, procarbazine, paclitaxel, pentostatin, semustine, streptozocin, tamoxifen, taxanes, taxol, testosterone propionate, thalidomide, thioguanine, thiotepa, teniposide, topotecan, uracil mustard, vinblastine, vinorelbine, vincristine.

The chemotherapeutic agent is preferably a low-molecular chemotherapeutic agent. The low-molecular chemotherapeutic agent is unlikely to interfere with the antibody even after its conjugation to the antibody. The low-molecular chemotherapeutic agent usually has a molecular weight of 100 to 2000, preferably 200 to 1000. All of the chemotherapeutic agents exemplified above are low-molecular chemotherapeutic agents. These chemotherapeutic agents encompass prodrugs that are converted in vivo to active chemotherapeutic agents. The prodrug activation may be an enzymatic conversion or a non-enzymatic conversion.

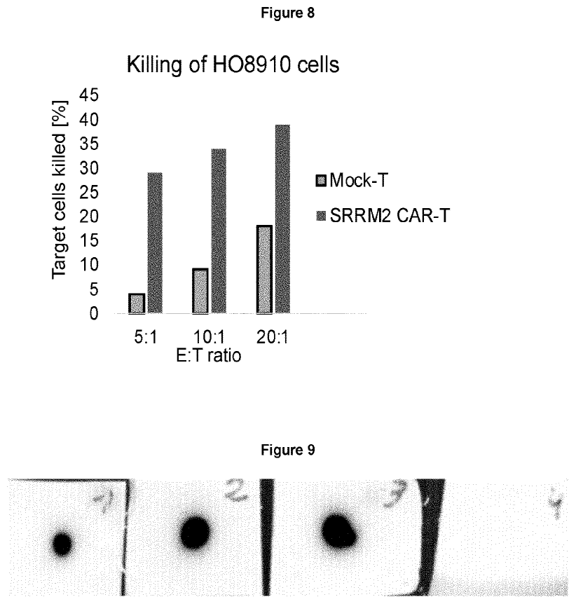

As shown in FIGS. 7 and 8, the surface presence of SRRM2 can be used for an SRRM2-specific CAR-T cell therapy. Thus, SRRM2-specific cancer therapy is enabled by the present invention. Accordingly, in one further embodiment of the pharmaceutical composition of the present invention, said antibody, which binds to human SRRM2 is part of a chimeric antigen receptor (CAR). For said embodiment, it is preferred that said CAR is comprised by a T cell, NK cell, NK-T cell or macrophage. Chimeric antigen receptors (CARs, also known as chimeric immunoreceptors, chimeric T cell receptors or artificial T cell receptors) are receptor proteins that have been engineered to give T cells the ability to target a specific protein.

In one embodiment of the pharmaceutical composition of the present invention, said antibody, which binds to human SRRM2, is less than 20% cross-reactive with SRRM2-related proteins. This means preferably that said antibody, which binds to human SRRM2, is less than 20% cross-reactive with other serine/arginine repetitive matrix proteins such as SRRM1, SRRM3, SRRM4 or SRRM5. More preferably, said antibody, which binds to human SRRM2 is less than 15% cross-reactive with SRRM2-related proteins, e.g. SRRM1, SRRM3, SRRM4 or SRRM5. Even more preferably, said antibody, which binds to human SRRM2, is less than 10% cross-reactive with SRRM2-related proteins, e.g. SRRM1, SRRM3, SRRM4 or SRRM5. Even more preferably, said antibody, which binds to human SRRM2, is less than 5% cross-reactive with SRRM2-related proteins, e.g. SRRM1, SRRM3, SRRM4 or SRRM5. Even more preferably, said antibody, which binds to human SRRM2, is less than 3% cross-reactive with SRRM2-related proteins, e.g. SRRM1, SRRM3, SRRM4 or SRRM5. Even more preferably, said antibody, which binds to human SRRM2, is less than 2% cross-reactive with SRRM2-related proteins, e.g. SRRM1, SRRM3, SRRM4 or SRRM5. Even more preferably, said antibody, which binds to human SRRM2, is less than 1% cross-reactive with SRRM2-related proteins, e.g. SRRM1, SRRM3, SRRM4 or SRRM5.

In one embodiment, said antibody, which binds to human SRRM2, is an antibody comprising a heavy chain variable region having an amino acid sequence with at least 85% identity to the amino acid sequence shown in SEQ ID NO: 1 and a light chain variable region having an amino acid sequence with at least 85% identity to the amino acid sequence shown in SEQ ID NO: 2 or an antibody, which binds to the same epitope.

In one embodiment, said antibody, which binds to human SRRM2, is an antibody comprising a heavy chain variable region having an amino acid sequence with at least 90% identity to the amino acid sequence shown in SEQ ID NO: 1 and a light chain variable region having an amino acid sequence with at least 90% identity to the amino acid sequence shown in SEQ ID NO: 2 or an antibody, which binds to the same epitope.

In one embodiment, said antibody, which binds to human SRRM2, is an antibody comprising a heavy chain variable region having an amino acid sequence with at least 95% identity to the amino acid sequence shown in SEQ ID NO: 1 and a light chain variable region having an amino acid sequence with at least 95% identity to the amino acid sequence shown in SEQ ID NO: 2 or an antibody, which binds to the same epitope.

In one embodiment, said antibody, which binds to human SRRM2, is an antibody comprising a heavy chain variable region having an amino acid sequence with at least 99% identity to the amino acid sequence shown in SEQ ID NO: 1 and a light chain variable region having an amino acid sequence with at least 99% identity to the amino acid sequence shown in SEQ ID NO: 2 or an antibody, which binds to the same epitope.

In one embodiment, said antibody, which binds to human SRRM2, is an antibody comprising a heavy chain variable region having an amino acid sequence with at least 85% identity to the amino acid sequence shown in SEQ ID NO: 3 and a light chain variable region having an amino acid sequence with at least 85% identity to the amino acid sequence shown in SEQ ID NO: 4 or an antibody, which binds to the same epitope.

In one embodiment, said antibody, which binds to human SRRM2, is an antibody comprising a heavy chain variable region having an amino acid sequence with at least 90% identity to the amino acid sequence shown in SEQ ID NO: 3 and a light chain variable region having an amino acid sequence with at least 90% identity to the amino acid sequence shown in SEQ ID NO: 4 or an antibody, which binds to the same epitope.

In one embodiment, said antibody, which binds to human SRRM2, is an antibody comprising a heavy chain variable region having an amino acid sequence with at least 95% identity to the amino acid sequence shown in SEQ ID NO: 3 and a light chain variable region having an amino acid sequence with at least 95% identity to the amino acid sequence shown in SEQ ID NO: 4 or an antibody, which binds to the same epitope.

In one embodiment, said antibody, which binds to human SRRM2 is an antibody comprising a heavy chain variable region having an amino acid sequence with at least 99% identity to the amino acid sequence shown in SEQ ID NO: 3 and a light chain variable region having an amino acid sequence with at least 99% identity to the amino acid sequence shown in SEQ ID NO: 4 or an antibody, which binds to the same epitope.

In one embodiment, said antibody, which binds to human SRRM2, is an antibody comprising a heavy chain variable region having an amino acid sequence with at least 85% identity to the amino acid sequence shown in SEQ ID NO: 5 and a light chain variable region having an amino acid sequence with at least 85% identity to the amino acid sequence shown in SEQ ID NO: 6 or an antibody, which binds to the same epitope.

In one embodiment, said antibody, which binds to human SRRM2, is an antibody comprising a heavy chain variable region having an amino acid sequence with at least 90% identity to the amino acid sequence shown in SEQ ID NO: 5 and a light chain variable region having an amino acid sequence with at least 90% identity to the amino acid sequence shown in SEQ ID NO: 6 or an antibody, which binds to the same epitope.

In one embodiment, said antibody, which binds to human SRRM2, is an antibody comprising a heavy chain variable region having an amino acid sequence with at least 95% identity to the amino acid sequence shown in SEQ ID NO: 5 and a light chain variable region having an amino acid sequence with at least 95% identity to the amino acid sequence shown in SEQ ID NO: 6 or an antibody, which binds to the same epitope.

In one embodiment, said antibody, which binds to human SRRM2, is an antibody comprising a heavy chain variable region having an amino acid sequence with at least 99% identity to the amino acid sequence shown in SEQ ID NO: 5 and a light chain variable region having an amino acid sequence with at least 99% identity to the amino acid sequence shown in SEQ ID NO: 6 or an antibody, which binds to the same epitope.

In one embodiment, said antibody, which binds to human SRRM2, is an antibody comprising a heavy chain variable region having an amino acid sequence with at least 85% identity to the amino acid sequence shown in SEQ ID NO: 5 and a light chain variable region having an amino acid sequence with at least 85% identity to the amino acid sequence shown in SEQ ID NO: 26 or an antibody, which binds to the same epitope.

In one embodiment, said antibody, which binds to human SRRM2, is an antibody comprising a heavy chain variable region having an amino acid sequence with at least 90% identity to the amino acid sequence shown in SEQ ID NO: 5 and a light chain variable region having an amino acid sequence with at least 90% identity to the amino acid sequence shown in SEQ ID NO: 26 or an antibody, which binds to the same epitope.

In one embodiment, said antibody, which binds to human SRRM2, is an antibody comprising a heavy chain variable region having an amino acid sequence with at least 95% identity to the amino acid sequence shown in SEQ ID NO: 5 and a light chain variable region having an amino acid sequence with at least 95% identity to the amino acid sequence shown in SEQ ID NO: 26 or an antibody, which binds to the same epitope.

In one embodiment, said antibody, which binds to human SRRM2, is an antibody comprising a heavy chain variable region having an amino acid sequence with at least 99% identity to the amino acid sequence shown in SEQ ID NO: 5 and a light chain variable region having an amino acid sequence with at least 99% identity to the amino acid sequence shown in SEQ ID NO: 26 or an antibody, which binds to the same epitope.

In one embodiment of the pharmaceutical composition of the present invention, said antibody, which binds to human SRRM2, is an antibody comprising a heavy chain variable region comprising heavy chain CDR1 having the amino acid sequence as set forth in SEQ ID NO: 7, heavy chain CDR2 having the amino acid sequence as set forth in SEQ ID NO: 8, and heavy chain CDR3 having the amino acid sequence as set forth in SEQ ID NO: 9, and a light chain variable region comprising light chain CDR1 having the amino acid sequence as set forth in SEQ ID NO: 10, light chain CDR2 having the amino acid sequence as set forth in SEQ ID NO: 11, and light chain CDR3 having the amino acid sequence as set forth in SEQ ID NO: 12, or an antibody, which binds to the same epitope.

In one embodiment of the pharmaceutical composition of the present invention, said antibody, which binds to human SRRM2 is an antibody comprising a heavy chain variable region comprising heavy chain CDR1 having the amino acid sequence as set forth in SEQ ID NO: 13, heavy chain CDR2 having the amino acid sequence as set forth in SEQ ID NO: 14, and heavy chain CDR3 having the amino acid sequence as set forth in SEQ ID NO: 15, and a light chain variable region comprising light chain CDR1 having the amino acid sequence as set forth in SEQ ID NO: 16, light chain CDR2 having the amino acid sequence as set forth in SEQ ID NO: 17, and light chain CDR3 having the amino acid sequence as set forth in SEQ ID NO: 18, or an antibody, which binds to the same epitope.

In one embodiment of the pharmaceutical composition of the present invention, said antibody, which binds to human SRRM2, is an antibody comprising a heavy chain variable region comprising heavy chain CDR1 having the amino acid sequence as set forth in SEQ ID NO: 19, heavy chain CDR2 having the amino acid sequence as set forth in SEQ ID NO: 20, and heavy chain CDR3 having the amino acid sequence as set forth in SEQ ID NO: 21, and a light chain variable region comprising light chain CDR1 having the amino acid sequence as set forth in SEQ ID NO: 22, light chain CDR2 having the amino acid sequence as set forth in SEQ ID NO: 23, and light chain CDR3 having the amino acid sequence as set forth in SEQ ID NO: 24, or an antibody, which binds to the same epitope.

In one embodiment of the pharmaceutical composition of the present invention, said antibody, which binds to human SRRM2, is an antibody comprising a heavy chain variable region comprising heavy chain CDR1 having the amino acid sequence as set forth in SEQ ID NO: 19, heavy chain CDR2 having the amino acid sequence as set forth in SEQ ID NO: 20, and heavy chain CDR3 having the amino acid sequence as set forth in SEQ ID NO: 21, and a light chain variable region comprising light chain CDR1 having the amino acid sequence as set forth in SEQ ID NO: 27, light chain CDR2 having the amino acid sequence as set forth in SEQ ID NO: 28, and light chain CDR3 having the amino acid sequence as set forth in SEQ ID NO: 29, or an antibody, which binds to the same epitope.

The antibodies described herein may contain constant regions. The constant regions used are not particularly limited, and any constant region may be used. Preferable examples of the constant regions used in the present invention can include human-derived constant regions. For example, a human IgG1-derived, human IgG2-derived, human IgG3-derived, or human IgG4-derived constant region can be used as a heavy chain constant region. Also, for example, human K chain-derived or human A chain-derived constant region can be used as a light chain constant region. The constant regions used in the present invention may be constant regions having a native sequence or may be modified constant regions having a sequence derived from the native sequence by the modification of one or more amino acids.

The antibodies described herein may also contain framework regions (FRs). The FRs used are not particularly limited, and any FR may be used as long as the resulting antibody maintains its binding activity to human SRRM2 present on the cell surface of a target cell. Preferable examples of the FRs used in the present invention can include human antibody-derived FRs. Since the technique of FR replacement with the antigen binding activity of an antibody maintained is known in the art, those skilled in the art can appropriately select FRs. The FRs used in the present invention may be FRs having a native sequence or may be FRs having a sequence derived from the native sequence by the modification of one or more amino acids.

Whether or not an antibody shares an epitope with a certain antibody can be confirmed based on their competition for the same epitope. The competition between the antibodies is detected by cross-blocking assay or the like. The cross-blocking assay is preferably, for example, competitive ELISA assay. Specifically, in the cross-blocking assay, SRRM2 proteins coated on the wells of a microtiter plate may be preincubated in the presence or absence of a candidate competing antibody and the antibody, which binds to human SRRM2 of the present invention is then added to the wells. The amount of the antibody of the present invention bound to the SRRM2 protein in the well indirectly correlates with the binding ability of the candidate competing antibody (antibody to be tested) that competes therewith for the binding to the same epitope. Specifically, the larger affinity the antibody to be tested has for the same epitope, the smaller amount of the antibody of the present invention is bound to the SRRM2 protein-coated well, while the larger amount of the antibody to be tested is bound to the SRRM2 protein-coated well.

The amount of the antibody bound to the well can be measured easily by labeling the antibody in advance. For example, a biotin-labeled antibody can be measured by use of an avidin-peroxidase conjugate and an appropriate substrate. The cross-blocking assay using enzyme (e.g., peroxidase) labeling is particularly referred to as competitive ELISA assay. The antibody can be labeled with other detectable or measurable labeling substances. Specifically, radio-labeling or fluorescent labeling or the like is known in the art.

Provided that a candidate antibody can bind to human SRRM2 present on the cell surface of a target cell by at least 80%, preferably at least 85%, more preferably at least 90%, even more preferably at least 95%, even more preferably at least 99%, compared to the binding activity obtained in a control test performed in the absence of the candidate antibody, this candidate antibody is determined as an antibody that binds to substantially the same epitope as that to which the antibody of the present invention binds or to which the antibody, which binds to human SRRM2 used in the pharmaceutical composition binds.

In a further aspect, the present invention provides the pharmaceutical composition according to the present invention for use in a method for the treatment of cancer in a human subject. It is preferred for said embodiment that said cancer is characterized by cells, wherein human SRRM2 is present on their cell surface. It is also preferred for said embodiment that said cancer is breast cancer, prostate cancer, lung cancer, colorectal cancer, ovarian cancer, pancreatic cancer, stomach cancer, cholangiogenic cancer, oral cavity squamous carcinoma, hematologic malignancy tumor, or glioma.

The pharmaceutical composition according to the present invention can be administered by standard routes. These include, but are not limited to: oral, rectal, ophthalmic (including intravitreal or intracameral), nasal, topical (including buccal and sublingual), intrauterine, vaginal or parenteral (including subcutaneous, intraperitoneal, intramuscular, intravenous, intradermal, intracranial, intratracheal, and epidural) transdermal, intraperitoneal, intracranial, intracerebroventricular, intracerebral, intravaginal, intrauterine, or parenteral (e.g., intravenous, intraspinal, subcutaneous or intramuscular) routes.

The present invention also comprises a method of treating cancer, wherein the method comprises administering a therapeutically effective amount of the pharmaceutical composition according to the present invention to a human subject. It is preferred for said embodiment that said cancer is characterized by cells, wherein human SRRM2 is present on their cell surface. It is also preferred for said embodiment that said cancer is breast cancer, prostate cancer, lung cancer, colorectal cancer, ovarian cancer, pancreatic cancer, stomach cancer, cholangiogenic cancer, oral cavity squamous carcinoma, hematologic malignancy tumor, or glioma.

Further, the present invention comprises a method of treating cancer, wherein the method comprises administering a therapeutically effective amount of the antibody, which binds to human SRRM2 described herein to a human subject. It is preferred for said embodiment that said cancer is characterized by cells, wherein human SRRM2 is present on their cell surface. It is also preferred for said embodiment that said cancer is breast cancer, prostate cancer, lung cancer, colorectal cancer, ovarian cancer, pancreatic cancer, stomach cancer, cholangiogenic cancer, oral cavity squamous carcinoma, hematologic malignancy tumor, or glioma.

The term “therapeutically effective amount” refers to an amount of the antibody or pharmaceutical composition according to the present invention or drug effective to “treat” cancer in the human subject. Specifically, in the case of cancer, the therapeutically effective amount of the antibody/pharmaceutical composition/drug can reduce the number of cancer cells; reduce the tumor size; inhibit or stop cancer cell infiltration into peripheral organs; inhibit and stop tumor metastasis; inhibit and stop tumor growth; relieve to some extent one or more of the symptoms associated with the cancer, or a combination of such effects on cancer cells. To the extent the antibody/pharmaceutical composition/drug prevents the growth and/or kills existing cancer cells, it can be referred to as cytostatic and/or cytotoxic.

The present invention also covers the use of the pharmaceutical composition according to the present invention for the manufacture of a medicament for the treatment of cancer.

In one embodiment, the present invention also covers the use of an antibody, which binds to human SRRM2 as described herein, for the manufacture of a medicament for the treatment of cancer.

Terms such as “treating” or “treatment” or “to treat” refer to both 1) therapeutic measures that cure, slow down, lessen symptoms of, and/or halt progression of a diagnosed pathologic condition or disorder and 2) prophylactic or preventative measures that prevent or slow the development of a targeted pathologic condition or disorder. Thus, those in need of treatment include those already with the disorder; those prone to have the disorder; and those in whom the disorder is to be prevented. A subject is successfully “treated” according to the methods of the present invention or with the pharmaceutical composition or antibody, which binds to human SRRM2 according to the present invention if the patient shows one or more of the following: a reduction in the number of or complete absence of cancer cells; a reduction in the tumor size; inhibition of or an absence of cancer cell infiltration into peripheral organs including the spread of cancer into soft tissue and bone; inhibition of or an absence of tumor metastasis; inhibition or an absence of tumor growth; relief of one or more symptoms associated with the specific cancer; reduced morbidity and mortality; and improvement in quality of life.

The antibody to be administered to humans can also be converted to a genetically recombinant antibody that has been engineered artificially, for example, for the purpose of reducing heteroantigenicity in humans. The genetically recombinant antibody encompasses, for example, chimeric antibodies and humanized antibodies as defined herein. These engineered antibodies can be produced using a method known in the art.

In a further aspect, the present invention provides a method for determining whether a human subject may suffer from cancer, comprising determining in a sample obtained from said human subject whether SRRM2 is present on the cell surface of (cancer) cells comprised by said sample.

As shown in Example 9, SRRM2 is also present on the surface of extracellular vesicles obtained from a subject diagnosed with cancer. Accordingly, the present invention further relates to a method for determining whether a human subject may suffer from cancer, comprising determining in a sample obtained from said human subject whether SRRM2 is present on the surface of extracellular vesicles. “Extracellular vesicles” (EV) as used herein may relate to lipid bilayer-delimited particles that are naturally released from almost all types of cell and, unlike a cell, cannot replicate. EVs typically range in diameter from near the size of the smallest physically possible unilamellar liposome (around 20-30 nanometers) to as large as 10 μm or more, although the vast majority of EVs are smaller than 200 nm.

EVs can be divided according to size and synthesis route into exosomes, microvesicles and apoptotic bodies. Accordingly, the EV is preferably selected from the group consisting of exosomes, microvesicles and apoptotic bodies. Usually, they carry a cargo of proteins, nucleic acids, lipids, metabolites, and even organelles from the parent cell. Diverse EV subtypes have been proposed. Accordingly, the EV may also be one or more of the following: ectosomes, microvesicles, microparticles, exosomes, oncosomes, apoptotic bodies, exomeres and the like. Preferably, the EV is an oncosome. In the context of EVs, the sample preferably is ascites fluid.

EVs may be isolated and/or enriched from the sample by using an antibody or antibody fragment that specifically binds to a biomarker present on the surface of an EV prior to determining in a sample obtained from said human subject whether SRRM2 is present on the surface of extracellular vesicles. Such biomarkers for EV include, e.g., for exosomes Alix, Tsg101, tetraspanins such as CD81, CD63 and CD9 or flotillin, for microvesicles integrins, selectins or CD40 and for apoptotic bodies Annexin V or phosphatidylserine. Antibodies against these markers are commercially available, e.g., from Abcam. Methods for isolating and/or enriching EVs are further described in Campos-Silva et al. (2019), Scientific Reports, 9:2042 (2019) or Pugholm et al. (2015), Biomed Res Int., 2015:524817, both of which are hereby incorporated by reference in its entirety.

Said pharmaceutical composition can also be a diagnostic composition, preferably in case it is used in the context of a method for determining whether a human subject may suffer from cancer or a neurodegenerative disease. Accordingly, the present invention also relates to a diagnostic composition, comprising an antibody, preferably a monoclonal antibody, which binds to human Serine/arginine repetitive matrix protein 2 (SRRM2) present on the cell surface of a target cell, preferably a cancer cell, and optionally a pharmaceutically acceptable carrier, diluent or excipient.

Said method for determining whether a human subject may suffer from cancer may comprise the following steps:
providing a sample obtained from said human subject,
determining in said sample obtained from said human subject whether SRRM2 is present on the cell surface of (cancer) cells comprised by said sample,
correlating the presence of SRRM2 on the cell surface of (cancer) cells comprised by said sample with determining whether the human subject may suffer from cancer.

A “subject” or “human subject” in the context of the present invention is a human being. For example, the subject may be a patient being suspected of having a disease or clinical condition associated with cancer or being diagnosed with such a disease or clinical condition. The subject may be a patient being suspected of having a disease or clinical condition associated with a neurodegenerative disease or being diagnosed with such a diseases or clinical condition.

Said “sample” obtained from said human subject is preferably selected from the group consisting of a blood sample, a serum sample, a plasma sample, a cerebrospinal fluid sample, a saliva sample, a solubilised tissue sample and an urine sample or an extract of any of the aforementioned samples. Preferably, the sample is a serum sample or a plasma sample. Most preferably, the sample is a serum sample for determining whether a human subject may suffer from cancer.

The step of “determining in said sample obtained from said human subject whether SRRM2 is present on the cell surface of target cells comprised by said sample” may comprise to determine the presence of SRRM2 or the fragments thereof by contacting the sample with at least one SRRM2 binder. The at least one binder may, for example, be the antibody according to the present invention or as described herein. It is preferred that at least one binder is less than 20% cross-reactive with other proteins, particularly other peroxiredoxins such as SRRM1, SRRM3, SRRM4 or SRRM5, more preferably less than 15%, more preferably less than 10%, more preferably less than 5%, even more preferably less than 3%, even more preferably less than 2% and even more preferably less than 1% cross reactive.

The present invention further relates to an antibody, which binds to human SRRM2, wherein said antibody is an antibody comprising a heavy chain variable region having an amino acid sequence with at least 85% identity to the amino acid sequence shown in SEQ ID NO: 1 and a light chain variable region having an amino acid sequence with at least 85% identity to the amino acid sequence shown in SEQ ID NO: 2 or an antibody, which binds to the same epitope.

The present invention further relates to an antibody, which binds to human SRRM2, wherein said antibody is an antibody comprising a heavy chain variable region having an amino acid sequence with at least 90% identity to the amino acid sequence shown in SEQ ID NO: 1 and a light chain variable region having an amino acid sequence with at least 90% identity to the amino acid sequence shown in SEQ ID NO: 2 or an antibody, which binds to the same epitope.

The present invention further relates to an antibody, which binds to human SRRM2, wherein said antibody is an antibody comprising a heavy chain variable region having an amino acid sequence with at least 95% identity to the amino acid sequence shown in SEQ ID NO: 1 and a light chain variable region having an amino acid sequence with at least 95% identity to the amino acid sequence shown in SEQ ID NO: 2 or an antibody, which binds to the same epitope.

The present invention further relates to an antibody, which binds to human SRRM2, wherein said antibody is an antibody comprising a heavy chain variable region having an amino acid sequence with at least 99% identity to the amino acid sequence shown in SEQ ID NO: 1 and a light chain variable region having an amino acid sequence with at least 99% identity to the amino acid sequence shown in SEQ ID NO: 2 or an antibody, which binds to the same epitope.

The present invention further relates to an antibody, which binds to human SRRM2, wherein said antibody is an antibody comprising a heavy chain variable region having an amino acid sequence with at least 85% identity to the amino acid sequence shown in SEQ ID NO: 3 and a light chain variable region having an amino acid sequence with at least 85% identity to the amino acid sequence shown in SEQ ID NO: 4 or an antibody, which binds to the same epitope.

The present invention further relates to an antibody, which binds to human SRRM2, wherein said antibody is an antibody comprising a heavy chain variable region having an amino acid sequence with at least 90% identity to the amino acid sequence shown in SEQ ID NO: 3 and a light chain variable region having an amino acid sequence with at least 90% identity to the amino acid sequence shown in SEQ ID NO: 4 or an antibody, which binds to the same epitope.

The present invention further relates to an antibody, which binds to human SRRM2, wherein said antibody is an antibody comprising a heavy chain variable region having an amino acid sequence with at least 95% identity to the amino acid sequence shown in SEQ ID NO: 3 and a light chain variable region having an amino acid sequence with at least 95% identity to the amino acid sequence shown in SEQ ID NO: 4 or an antibody, which binds to the same epitope.

The present invention further relates to an antibody, which binds to human SRRM2, wherein said antibody is an antibody comprising a heavy chain variable region having an amino acid sequence with at least 99% identity to the amino acid sequence shown in SEQ ID NO: 3 and a light chain variable region having an amino acid sequence with at least 99% identity to the amino acid sequence shown in SEQ ID NO: 4 or an antibody, which binds to the same epitope.

The present invention further relates to an antibody, which binds to human SRRM2, wherein said antibody is an antibody comprising a heavy chain variable region having an amino acid sequence with at least 85% identity to the amino acid sequence shown in SEQ ID NO: 5 and a light chain variable region having an amino acid sequence with at least 85% identity to the amino acid sequence shown in SEQ ID NO: 6 or an antibody, which binds to the same epitope.

The present invention further relates to an antibody, which binds to human SRRM2, wherein said antibody is an antibody comprising a heavy chain variable region having an amino acid sequence with at least 90% identity to the amino acid sequence shown in SEQ ID NO: 5 and a light chain variable region having an amino acid sequence with at least 90% identity to the amino acid sequence shown in SEQ ID NO: 6 or an antibody, which binds to the same epitope.

The present invention further relates to an antibody, which binds to human SRRM2, wherein said antibody is an antibody comprising a heavy chain variable region having an amino acid sequence with at least 95% identity to the amino acid sequence shown in SEQ ID NO: 5 and a light chain variable region having an amino acid sequence with at least 95% identity to the amino acid sequence shown in SEQ ID NO: 6 or an antibody, which binds to the same epitope.

The present invention further relates to an antibody, which binds to human SRRM2, wherein said antibody is an antibody comprising a heavy chain variable region having an amino acid sequence with at least 99% identity to the amino acid sequence shown in SEQ ID NO: 5 and a light chain variable region having an amino acid sequence with at least 99% identity to the amino acid sequence shown in SEQ ID NO: 6 or an antibody, which binds to the same epitope.

The present invention further relates to an antibody, which binds to human SRRM2, wherein said antibody is an antibody comprising a heavy chain variable region having an amino acid sequence with at least 85% identity to the amino acid sequence shown in SEQ ID NO: 5 and a light chain variable region having an amino acid sequence with at least 85% identity to the amino acid sequence shown in SEQ ID NO: 26 or an antibody, which binds to the same epitope.

The present invention further relates to an antibody, which binds to human SRRM2, wherein said antibody is an antibody comprising a heavy chain variable region having an amino acid sequence with at least 90% identity to the amino acid sequence shown in SEQ ID NO: 5 and a light chain variable region having an amino acid sequence with at least 90% identity to the amino acid sequence shown in SEQ ID NO: 26 or an antibody, which binds to the same epitope.

The present invention further relates to an antibody, which binds to human SRRM2, wherein said antibody is an antibody comprising a heavy chain variable region having an amino acid sequence with at least 95% identity to the amino acid sequence shown in SEQ ID NO: 5 and a light chain variable region having an amino acid sequence with at least 95% identity to the amino acid sequence shown in SEQ ID NO: 26 or an antibody, which binds to the same epitope.

The present invention further relates to an antibody, which binds to human SRRM2, wherein said antibody is an antibody comprising a heavy chain variable region having an amino acid sequence with at least 99% identity to the amino acid sequence shown in SEQ ID NO: 5 and a light chain variable region having an amino acid sequence with at least 99% identity to the amino acid sequence shown in SEQ ID NO: 26 or an antibody, which binds to the same epitope.

The present invention further relates to an antibody, which binds to human SRRM2, wherein said antibody is an antibody comprising a heavy chain variable region comprising heavy chain CDR1 having the amino acid sequence as set forth in SEQ ID NO: 7, heavy chain CDR2 having the amino acid sequence as set forth in SEQ ID NO: 8, and heavy chain CDR3 having the amino acid sequence as set forth in SEQ ID NO: 9, and a light chain variable region comprising light chain CDR1 having the amino acid sequence as set forth in SEQ ID NO: 10, light chain CDR2 having the amino acid sequence as set forth in SEQ ID NO: 11, and light chain CDR3 having the amino acid sequence as set forth in SEQ ID NO: 12, or an antibody, which binds to the same epitope.

The present invention further relates to an antibody, which binds to human SRRM2, wherein said antibody is an antibody comprising a heavy chain variable region comprising heavy chain CDR1 having the amino acid sequence as set forth in SEQ ID NO: 13, heavy chain CDR2 having the amino acid sequence as set forth in SEQ ID NO: 14, and heavy chain CDR3 having the amino acid sequence as set forth in SEQ ID NO: 15, and a light chain variable region comprising light chain CDR1 having the amino acid sequence as set forth in SEQ ID NO: 16, light chain CDR2 having the amino acid sequence as set forth in SEQ ID NO: 17, and light chain CDR3 having the amino acid sequence as set forth in SEQ ID NO: 18, or an antibody, which binds to the same epitope.

The present invention further relates to an antibody, which binds to human SRRM2, wherein said antibody is an antibody comprising a heavy chain variable region comprising heavy chain CDR1 having the amino acid sequence as set forth in SEQ ID NO: 19, heavy chain CDR2 having the amino acid sequence as set forth in SEQ ID NO: 20, and heavy chain CDR3 having the amino acid sequence as set forth in SEQ ID NO: 21, and a light chain variable region comprising light chain CDR1 having the amino acid sequence as set forth in SEQ ID NO: 22, light chain CDR2 having the amino acid sequence as set forth in SEQ ID NO: 23, and light chain CDR3 having the amino acid sequence as set forth in SEQ ID NO: 24, or an antibody, which binds to the same epitope.

The present invention further relates to an antibody, which binds to human SRRM2, wherein said antibody is an antibody comprising a heavy chain variable region comprising heavy chain CDR1 having the amino acid sequence as set forth in SEQ ID NO: 19, heavy chain CDR2 having the amino acid sequence as set forth in SEQ ID NO: 20, and heavy chain CDR3 having the amino acid sequence as set forth in SEQ ID NO: 21, and a light chain variable region comprising light chain CDR1 having the amino acid sequence as set forth in SEQ ID NO: 27, light chain CDR2 having the amino acid sequence as set forth in SEQ ID NO: 28, and light chain CDR3 having the amino acid sequence as set forth in SEQ ID NO: 29, or an antibody, which binds to the same epitope.

The present invention also provides the use of the antibody, which binds to human SRRM2 of the present invention in the manufacture of a medicament for the treatment of cancer.

The invention additionally provides the antibody, which binds to human SRRM2, of the present invention for use in a method of treating cancer.

In some embodiments, the antibody, which binds to human SRRM2, of the present invention or described herein may contain human Fc regions that are modified to enhance effector function, for example, antigen-dependent cell-mediated cytotoxicity (ADCC) and/or complement dependent cytotoxicity (CDC). This can be achieved by introducing one or more amino acid substitutions in a Fc region of the antibody. For example, cysteine residue(s) can be introduced in the Fc region to allow interchain disulfide bond formation in this region to improve complement-mediated cell killing and antibody-dependent cellular cytotoxicity (ADCC). Homodimeric antibodies with enhanced anti-tumor activity can also be prepared using heterobifunctional cross-linkers as known to the person skilled in the art. Alternatively, an antibody may be engineered, which has dual Fc regions.

Furthermore, the antibodies as described herein may have substitution, deletion, addition and/or insertion of one or more amino acids in their CDR sequences as long as the resulting antibodies are functionally equivalent to the antibodies (a) to (d). The term “functionally equivalent” refers to being comparable in avidity for SRRM2 and cytotoxicity. The term “equivalent” refers to having at least 50%, preferably having at least 60%, more preferably having at least 70%, more preferably having at least 80%, even more preferably at least 90%, even more preferably at least 95% and even more preferably at least 99% activity, compared with the antibodies (a) to (d). The upper limit of the activity is not particularly limited and may be higher than that of the antibodies (a) to (d). The avidity or cytotoxicity can be assayed by a method generally known by those skilled in the art.

The presence of SRRM2 on the cell surface of a target cell or extracellular vesicles can also be indicative that a subject, from whom a sample comprising target cells has been obtained, suffers from a neurodegenerative disease. In this context, the target cell preferably is a neuron. The target cell may also be a glial cell. Glial cells include, but are not limited to, astrocytes, oligodendrocytes, ependymal cells, radial glia, Schwann cells, satellite cells, enteric glial cells, and microglia. In this context, the sample preferably is obtained from brain tissue, neuronal tissue and/or cerebrospinal fluid.

A “neurodegenerative disease” as used herein is characterized by the progressive loss of structure or function of neurons, in the process known as neurodegeneration. Such neuronal damage may ultimately involve cell death. Neurodegenerative diseases include, but are not limited to, amyotrophic lateral sclerosis, multiple sclerosis, Parkinson's disease, Alzheimer's disease, Huntington's disease, Batten disease, multiple system atrophy, and prion diseases.

Accordingly, the present invention also relates to the pharmaceutical composition of the invention for use in a method for the treatment of a neurodegenerative disease in a human subject. The present invention further relates to the antibody, which binds to human SRRM2, as used herein for use in a method for the treatment of a neurodegenerative disease in a human subject. Preferably, said neurodegenerative disease is characterized by cells, wherein human SRRM2 is present on their cell surface.

The present invention also comprises a method of treating a neurodegenerative disease, wherein the method comprises administering a therapeutically effective amount of the pharmaceutical composition according to the present invention to a human subject. Further, the present invention comprises a method of treating a neurodegenerative disease, wherein the method comprises administering a therapeutically effective amount of the antibody, which binds to human SRRM2, described herein to a human subject. It is preferred for said embodiment that said neurodegenerative disease is characterized by cells, wherein human SRRM2 is present on their cell surface.

The present invention further relates to a method for determining whether a human subject may suffer from a neurodegenerative disease, comprising determining in a sample obtained from said human subject whether SRRM2 is present on the cell surface of cells comprised by said sample.

The present invention further relates to a method for determining whether a human subject may suffer from a neurodegenerative disease, comprising determining in a sample obtained from said human subject whether SRRM2 is present on the surface of extracellular vesicles.

Said method for determining whether a human subject may suffer from a neurodegenerative disease may comprise the following steps:
providing a sample obtained from said human subject,
determining in said sample obtained from said human subject whether SRRM2 is present on the cell surface of target cells comprised by said sample,
correlating the presence of SRRM2 on the cell surface of said target cells comprised by said sample with determining whether the human subject may suffer from a neurodegenerative disease.

The present invention further relates to an antibody, which binds to human serine/arginine repetitive matrix protein 2 (SRRM2) present on the cell surface of a target cell for use in a method of killing said target cell having SRRM2 present on the cell surface.

In one embodiment of the antibody for use in a method of killing said target cell having SRRM2 present on the cell surface, said antibody is an antibody comprising a heavy chain variable region having an amino acid sequence with at least 85% identity to the amino acid sequence shown in SEQ ID NO: 1 and a light chain variable region having an amino acid sequence with at least 85% identity to the amino acid sequence shown in SEQ ID NO: 2 or an antibody, which binds to the same epitope.

In one embodiment of the antibody for use in a method of killing said target cell having SRRM2 present on the cell surface, said antibody is an antibody comprising a heavy chain variable region having an amino acid sequence with at least 90% identity to the amino acid sequence shown in SEQ ID NO: 1 and a light chain variable region having an amino acid sequence with at least 90% identity to the amino acid sequence shown in SEQ ID NO: 2 or an antibody, which binds to the same epitope.

In one embodiment of the antibody for use in a method of killing said target cell having SRRM2 present on the cell surface, said antibody is an antibody comprising a heavy chain variable region having an amino acid sequence with at least 95% identity to the amino acid sequence shown in SEQ ID NO: 1 and a light chain variable region having an amino acid sequence with at least 95% identity to the amino acid sequence shown in SEQ ID NO: 2 or an antibody, which binds to the same epitope.

In one embodiment of the antibody for use in a method of killing said target cell having SRRM2 present on the cell surface, said antibody is an antibody comprising a heavy chain variable region having an amino acid sequence with at least 99% identity to the amino acid sequence shown in SEQ ID NO: 1 and a light chain variable region having an amino acid sequence with at least 99% identity to the amino acid sequence shown in SEQ ID NO: 2 or an antibody, which binds to the same epitope.

In one embodiment of the antibody for use in a method of killing said target cell having SRRM2 present on the cell surface, said antibody is an antibody comprising a heavy chain variable region having an amino acid sequence with at least 85% identity to the amino acid sequence shown in SEQ ID NO: 3 and a light chain variable region having an amino acid sequence with at least 85% identity to the amino acid sequence shown in SEQ ID NO: 4 or an antibody, which binds to the same epitope.

In one embodiment of the antibody for use in a method of killing said target cell having SRRM2 present on the cell surface, said antibody is an antibody comprising a heavy chain variable region having an amino acid sequence with at least 90% identity to the amino acid sequence shown in SEQ ID NO: 3 and a light chain variable region having an amino acid sequence with at least 90% identity to the amino acid sequence shown in SEQ ID NO: 4 or an antibody, which binds to the same epitope.

In one embodiment of the antibody for use in a method of killing said target cell having SRRM2 present on the cell surface, said antibody is an antibody comprising a heavy chain variable region having an amino acid sequence with at least 95% identity to the amino acid sequence shown in SEQ ID NO: 3 and a light chain variable region having an amino acid sequence with at least 95% identity to the amino acid sequence shown in SEQ ID NO: 4 or an antibody, which binds to the same epitope.

In one embodiment of the antibody for use in a method of killing said target cell having SRRM2 present on the cell surface, said antibody is an antibody comprising a heavy chain variable region having an amino acid sequence with at least 99% identity to the amino acid sequence shown in SEQ ID NO: 3 and a light chain variable region having an amino acid sequence with at least 99% identity to the amino acid sequence shown in SEQ ID NO: 4 or an antibody, which binds to the same epitope.

In one embodiment of the antibody for use in a method of killing said target cell having SRRM2 present on the cell surface, said antibody is an antibody comprising a heavy chain variable region having an amino acid sequence with at least 85% identity to the amino acid sequence shown in SEQ ID NO: 5 and a light chain variable region having an amino acid sequence with at least 85% identity to the amino acid sequence shown in SEQ ID NO: 6 or an antibody, which binds to the same epitope.

In one embodiment of the antibody for use in a method of killing said target cell having SRRM2 present on the cell surface, said antibody is an antibody comprising a heavy chain variable region having an amino acid sequence with at least 90% identity to the amino acid sequence shown in SEQ ID NO: 5 and a light chain variable region having an amino acid sequence with at least 90% identity to the amino acid sequence shown in SEQ ID NO: 6 or an antibody, which binds to the same epitope.

In one embodiment of the antibody for use in a method of killing said target cell having SRRM2 present on the cell surface, said antibody is an antibody comprising a heavy chain variable region having an amino acid sequence with at least 95% identity to the amino acid sequence shown in SEQ ID NO: 5 and a light chain variable region having an amino acid sequence with at least 95% identity to the amino acid sequence shown in SEQ ID NO: 6 or an antibody, which binds to the same epitope.

In one embodiment of the antibody for use in a method of killing said target cell having SRRM2 present on the cell surface, said antibody is an antibody comprising a heavy chain variable region having an amino acid sequence with at least 99% identity to the amino acid sequence shown in SEQ ID NO: 5 and a light chain variable region having an amino acid sequence with at least 99% identity to the amino acid sequence shown in SEQ ID NO: 6 or an antibody, which binds to the same epitope.

In one embodiment of the antibody for use in a method of killing said target cell having SRRM2 present on the cell surface, said antibody is an antibody comprising a heavy chain variable region having an amino acid sequence with at least 85% identity to the amino acid sequence shown in SEQ ID NO: 5 and a light chain variable region having an amino acid sequence with at least 85% identity to the amino acid sequence shown in SEQ ID NO: 26 or an antibody, which binds to the same epitope.

In one embodiment of the antibody for use in a method of killing said target cell having SRRM2 present on the cell surface, said antibody is an antibody comprising a heavy chain variable region having an amino acid sequence with at least 90% identity to the amino acid sequence shown in SEQ ID NO: 5 and a light chain variable region having an amino acid sequence with at least 90% identity to the amino acid sequence shown in SEQ ID NO: 26 or an antibody, which binds to the same epitope.

In one embodiment of the antibody for use in a method of killing said target cell having SRRM2 present on the cell surface, said antibody is an antibody comprising a heavy chain variable region having an amino acid sequence with at least 95% identity to the amino acid sequence shown in SEQ ID NO: 5 and a light chain variable region having an amino acid sequence with at least 95% identity to the amino acid sequence shown in SEQ ID NO: 26 or an antibody, which binds to the same epitope.

In one embodiment of the antibody for use in a method of killing said target cell having SRRM2 present on the cell surface, said antibody is an antibody comprising a heavy chain variable region having an amino acid sequence with at least 99% identity to the amino acid sequence shown in SEQ ID NO: 5 and a light chain variable region having an amino acid sequence with at least 99% identity to the amino acid sequence shown in SEQ ID NO: 26 or an antibody, which binds to the same epitope.

In one embodiment of the antibody for use in a method of killing said target cell having SRRM2 present on the cell surface, said antibody is an antibody comprising a heavy chain variable region comprising heavy chain CDR1 having the amino acid sequence as set forth in SEQ ID NO: 7, heavy chain CDR2 having the amino acid sequence as set forth in SEQ ID NO: 8, and heavy chain CDR3 having the amino acid sequence as set forth in SEQ ID NO: 9, and a light chain variable region comprising light chain CDR1 having the amino acid sequence as set forth in SEQ ID NO: 10, light chain CDR2 having the amino acid sequence as set forth in SEQ ID NO: 11, and light chain CDR3 having the amino acid sequence as set forth in SEQ ID NO: 12, or an antibody, which binds to the same epitope.

In one embodiment of the antibody for use in a method of killing said target cell having SRRM2 present on the cell surface, said antibody is an antibody comprising a heavy chain variable region comprising heavy chain CDR1 having the amino acid sequence as set forth in SEQ ID NO: 13, heavy chain CDR2 having the amino acid sequence as set forth in SEQ ID NO: 14, and heavy chain CDR3 having the amino acid sequence as set forth in SEQ ID NO: 15, and a light chain variable region comprising light chain CDR1 having the amino acid sequence as set forth in SEQ ID NO: 16, light chain CDR2 having the amino acid sequence as set forth in SEQ ID NO: 17, and light chain CDR3 having the amino acid sequence as set forth in SEQ ID NO: 18, or an antibody, which binds to the same epitope.

In one embodiment of the antibody for use in a method of killing said target cell having SRRM2 present on the cell surface, said antibody is an antibody comprising a heavy chain variable region comprising heavy chain CDR1 having the amino acid sequence as set forth in SEQ ID NO: 19, heavy chain CDR2 having the amino acid sequence as set forth in SEQ ID NO: 20, and heavy chain CDR3 having the amino acid sequence as set forth in SEQ ID NO: 21, and a light chain variable region comprising light chain CDR1 having the amino acid sequence as set forth in SEQ ID NO: 22, light chain CDR2 having the amino acid sequence as set forth in SEQ ID NO: 23, and light chain CDR3 having the amino acid sequence as set forth in SEQ ID NO: 24, or an antibody, which binds to the same epitope.

In one embodiment of the antibody for use in a method of killing said target cell having SRRM2 present on the cell surface, said antibody is an antibody comprising a heavy chain variable region comprising heavy chain CDR1 having the amino acid sequence as set forth in SEQ ID NO: 19, heavy chain CDR2 having the amino acid sequence as set forth in SEQ ID NO: 20, and heavy chain CDR3 having the amino acid sequence as set forth in SEQ ID NO: 21, and a light chain variable region comprising light chain CDR1 having the amino acid sequence as set forth in SEQ ID NO: 27, light chain CDR2 having the amino acid sequence as set forth in SEQ ID NO: 28, and light chain CDR3 having the amino acid sequence as set forth in SEQ ID NO: 29, or an antibody, which binds to the same epitope.

All embodiments, which have been described herein for the pharmaceutical composition of the present invention and the antibody, which binds to human SRRM2 present on the cell surface of a target cell of the present invention and the methods of the present invention as described herein as well as the respective definitions as described above, also apply for the antibody for use in a method of killing said target cell having SRRM2 present on the cell surface of the present invention as described herein.

The present invention also relates to a method of killing a target cell having serine/arginine repetitive matrix protein 2 (SRRM2) present on the cell surface of said target cell, comprising administering an antibody, which binds to human SRRM2 present on the cell surface of said target cell.

In one embodiment of the method of killing a target cell having serine/arginine repetitive matrix protein 2 (SRRM2) present on the cell surface of said target cell, said antibody is an antibody comprising a heavy chain variable region having an amino acid sequence with at least 85% identity to the amino acid sequence shown in SEQ ID NO: 1 and a light chain variable region having an amino acid sequence with at least 85% identity to the amino acid sequence shown in SEQ ID NO: 2 or an antibody, which binds to the same epitope.

In one embodiment of the method of killing a target cell having serine/arginine repetitive matrix protein 2 (SRRM2) present on the cell surface of said target cell, said antibody is an antibody comprising a heavy chain variable region having an amino acid sequence with at least 90% identity to the amino acid sequence shown in SEQ ID NO: 1 and a light chain variable region having an amino acid sequence with at least 90% identity to the amino acid sequence shown in SEQ ID NO: 2 or an antibody, which binds to the same epitope.

In one embodiment of the method of killing a target cell having serine/arginine repetitive matrix protein 2 (SRRM2) present on the cell surface of said target cell, said antibody is an antibody comprising a heavy chain variable region having an amino acid sequence with at least 95% identity to the amino acid sequence shown in SEQ ID NO: 1 and a light chain variable region having an amino acid sequence with at least 95% identity to the amino acid sequence shown in SEQ ID NO: 2 or an antibody, which binds to the same epitope.

In one embodiment of the method of killing a target cell having serine/arginine repetitive matrix protein 2 (SRRM2) present on the cell surface of said target cell, said antibody is an antibody comprising a heavy chain variable region having an amino acid sequence with at least 99% identity to the amino acid sequence shown in SEQ ID NO: 1 and a light chain variable region having an amino acid sequence with at least 99% identity to the amino acid sequence shown in SEQ ID NO: 2 or an antibody, which binds to the same epitope.

In one embodiment of the method of killing a target cell having serine/arginine repetitive matrix protein 2 (SRRM2) present on the cell surface of said target cell, said antibody is an antibody comprising a heavy chain variable region having an amino acid sequence with at least 85% identity to the amino acid sequence shown in SEQ ID NO: 3 and a light chain variable region having an amino acid sequence with at least 85% identity to the amino acid sequence shown in SEQ ID NO: 4 or an antibody, which binds to the same epitope.

In one embodiment of the method of killing a target cell having serine/arginine repetitive matrix protein 2 (SRRM2) present on the cell surface of said target cell, said antibody is an antibody comprising a heavy chain variable region having an amino acid sequence with at least 90% identity to the amino acid sequence shown in SEQ ID NO: 3 and a light chain variable region having an amino acid sequence with at least 90% identity to the amino acid sequence shown in SEQ ID NO: 4 or an antibody, which binds to the same epitope.

In one embodiment of the method of killing a target cell having serine/arginine repetitive matrix protein 2 (SRRM2) present on the cell surface of said target cell, said antibody is an antibody comprising a heavy chain variable region having an amino acid sequence with at least 95% identity to the amino acid sequence shown in SEQ ID NO: 3 and a light chain variable region having an amino acid sequence with at least 95% identity to the amino acid sequence shown in SEQ ID NO: 4 or an antibody, which binds to the same epitope.

In one embodiment of the method of killing a target cell having serine/arginine repetitive matrix protein 2 (SRRM2) present on the cell surface of said target cell, said antibody is an antibody comprising a heavy chain variable region having an amino acid sequence with at least 99% identity to the amino acid sequence shown in SEQ ID NO: 3 and a light chain variable region having an amino acid sequence with at least 99% identity to the amino acid sequence shown in SEQ ID NO: 4 or an antibody, which binds to the same epitope.

In one embodiment of the method of killing a target cell having serine/arginine repetitive matrix protein 2 (SRRM2) present on the cell surface of said target cell, said antibody is an antibody comprising a heavy chain variable region having an amino acid sequence with at least 85% identity to the amino acid sequence shown in SEQ ID NO: 5 and a light chain variable region having an amino acid sequence with at least 85% identity to the amino acid sequence shown in SEQ ID NO: 6 or an antibody, which binds to the same epitope.

In one embodiment of the method of killing a target cell having serine/arginine repetitive matrix protein 2 (SRRM2) present on the cell surface of said target cell, said antibody is an antibody comprising a heavy chain variable region having an amino acid sequence with at least 90% identity to the amino acid sequence shown in SEQ ID NO: 5 and a light chain variable region having an amino acid sequence with at least 90% identity to the amino acid sequence shown in SEQ ID NO: 6 or an antibody, which binds to the same epitope.

In one embodiment of the method of killing a target cell having serine/arginine repetitive matrix protein 2 (SRRM2) present on the cell surface of said target cell, said antibody is an antibody comprising a heavy chain variable region having an amino acid sequence with at least 95% identity to the amino acid sequence shown in SEQ ID NO: 5 and a light chain variable region having an amino acid sequence with at least 95% identity to the amino acid sequence shown in SEQ ID NO: 6 or an antibody, which binds to the same epitope.

In one embodiment of the method of killing a target cell having serine/arginine repetitive matrix protein 2 (SRRM2) present on the cell surface of said target cell, said antibody is an antibody comprising a heavy chain variable region having an amino acid sequence with at least 99% identity to the amino acid sequence shown in SEQ ID NO: 5 and a light chain variable region having an amino acid sequence with at least 99% identity to the amino acid sequence shown in SEQ ID NO: 6 or an antibody, which binds to the same epitope.

In one embodiment of the method of killing a target cell having serine/arginine repetitive matrix protein 2 (SRRM2) present on the cell surface of said target cell, said antibody is an antibody comprising a heavy chain variable region having an amino acid sequence with at least 85% identity to the amino acid sequence shown in SEQ ID NO: 5 and a light chain variable region having an amino acid sequence with at least 85% identity to the amino acid sequence shown in SEQ ID NO: 26 or an antibody, which binds to the same epitope.

In one embodiment of the method of killing a target cell having serine/arginine repetitive matrix protein 2 (SRRM2) present on the cell surface of said target cell, said antibody is an antibody comprising a heavy chain variable region having an amino acid sequence with at least 90% identity to the amino acid sequence shown in SEQ ID NO: 5 and a light chain variable region having an amino acid sequence with at least 90% identity to the amino acid sequence shown in SEQ ID NO: 26 or an antibody, which binds to the same epitope.

In one embodiment of the method of killing a target cell having serine/arginine repetitive matrix protein 2 (SRRM2) present on the cell surface of said target cell, said antibody is an antibody comprising a heavy chain variable region having an amino acid sequence with at least 95% identity to the amino acid sequence shown in SEQ ID NO: 5 and a light chain variable region having an amino acid sequence with at least 95% identity to the amino acid sequence shown in SEQ ID NO: 26 or an antibody, which binds to the same epitope.

In one embodiment of the method of killing a target cell having serine/arginine repetitive matrix protein 2 (SRRM2) present on the cell surface of said target cell, said antibody is an antibody comprising a heavy chain variable region having an amino acid sequence with at least 99% identity to the amino acid sequence shown in SEQ ID NO: 5 and a light chain variable region having an amino acid sequence with at least 99% identity to the amino acid sequence shown in SEQ ID NO: 26 or an antibody, which binds to the same epitope.

In one embodiment of the method of killing a target cell having serine/arginine repetitive matrix protein 2 (SRRM2) present on the cell surface of said target cell, said antibody is an antibody comprising a heavy chain variable region comprising heavy chain CDR1 having the amino acid sequence as set forth in SEQ ID NO: 7, heavy chain CDR2 having the amino acid sequence as set forth in SEQ ID NO: 8, and heavy chain CDR3 having the amino acid sequence as set forth in SEQ ID NO: 9, and a light chain variable region comprising light chain CDR1 having the amino acid sequence as set forth in SEQ ID NO: 10, light chain CDR2 having the amino acid sequence as set forth in SEQ ID NO: 11, and light chain CDR3 having the amino acid sequence as set forth in SEQ ID NO: 12, or an antibody, which binds to the same epitope.

In one embodiment of the method of killing a target cell having serine/arginine repetitive matrix protein 2 (SRRM2) present on the cell surface of said target cell, said antibody is an antibody comprising a heavy chain variable region comprising heavy chain CDR1 having the amino acid sequence as set forth in SEQ ID NO: 13, heavy chain CDR2 having the amino acid sequence as set forth in SEQ ID NO: 14, and heavy chain CDR3 having the amino acid sequence as set forth in SEQ ID NO: 15, and a light chain variable region comprising light chain CDR1 having the amino acid sequence as set forth in SEQ ID NO: 16, light chain CDR2 having the amino acid sequence as set forth in SEQ ID NO: 17, and light chain CDR3 having the amino acid sequence as set forth in SEQ ID NO: 18, or an antibody, which binds to the same epitope.

In one embodiment of the method of killing a target cell having serine/arginine repetitive matrix protein 2 (SRRM2) present on the cell surface of said target cell, said antibody is an antibody comprising a heavy chain variable region comprising heavy chain CDR1 having the amino acid sequence as set forth in SEQ ID NO: 19, heavy chain CDR2 having the amino acid sequence as set forth in SEQ ID NO: 20, and heavy chain CDR3 having the amino acid sequence as set forth in SEQ ID NO: 21, and a light chain variable region comprising light chain CDR1 having the amino acid sequence as set forth in SEQ ID NO: 22, light chain CDR2 having the amino acid sequence as set forth in SEQ ID NO: 23, and light chain CDR3 having the amino acid sequence as set forth in SEQ ID NO: 24, or an antibody, which binds to the same epitope.

In one embodiment of the method of killing a target cell having serine/arginine repetitive matrix protein 2 (SRRM2) present on the cell surface of said target cell, said antibody is an antibody comprising a heavy chain variable region comprising heavy chain CDR1 having the amino acid sequence as set forth in SEQ ID NO: 19, heavy chain CDR2 having the amino acid sequence as set forth in SEQ ID NO: 20, and heavy chain CDR3 having the amino acid sequence as set forth in SEQ ID NO: 21, and a light chain variable region comprising light chain CDR1 having the amino acid sequence as set forth in SEQ ID NO: 27, light chain CDR2 having the amino acid sequence as set forth in SEQ ID NO: 28, and light chain CDR3 having the amino acid sequence as set forth in SEQ ID NO: 29, or an antibody, which binds to the same epitope.

All embodiments, which are described herein for the pharmaceutical composition of the present invention, the antibody, which binds to human SRRM2 present on the cell surface of a target cell of the present invention, the method of determining whether a human subject may suffer from cancer of the present invention as described herein as well as the respective definitions as described above, also apply for the method of killing a target cell having serine/arginine repetitive matrix protein 2 (SRRM2) present on the cell surface of said target cell as described herein.

The present invention further relates to an antibody, which binds to human serine/arginine repetitive matrix protein 2 (SRRM2) present on the cell surface of a target cell for use in a method for the treatment of cancer in a subject, comprising, prior to the treatment of cancer in said subject, determining whether a target cell of said subject has SRRM2 present on the cell surface.

In one embodiment of the antibody for use in a method for the treatment of cancer in a subject, comprising, prior to the treatment of cancer in said subject, determining whether a target cell of said subject has SRRM2 present on the cell surface, said antibody is an antibody comprising a heavy chain variable region having an amino acid sequence with at least 85% identity to the amino acid sequence shown in SEQ ID NO: 1 and a light chain variable region having an amino acid sequence with at least 85% identity to the amino acid sequence shown in SEQ ID NO: 2 or an antibody, which binds to the same epitope.

In one embodiment of the antibody for use in a method for the treatment of cancer in a subject, comprising, prior to the treatment of cancer in said subject, determining whether a target cell of said subject has SRRM2 present on the cell surface, said antibody is an antibody comprising a heavy chain variable region having an amino acid sequence with at least 90% identity to the amino acid sequence shown in SEQ ID NO: 1 and a light chain variable region having an amino acid sequence with at least 90% identity to the amino acid sequence shown in SEQ ID NO: 2 or an antibody, which binds to the same epitope.

In one embodiment of the antibody for use in a method for the treatment of cancer in a subject, comprising, prior to the treatment of cancer in said subject, determining whether a target cell of said subject has SRRM2 present on the cell surface, said antibody is an antibody comprising a heavy chain variable region having an amino acid sequence with at least 95% identity to the amino acid sequence shown in SEQ ID NO: 1 and a light chain variable region having an amino acid sequence with at least 95% identity to the amino acid sequence shown in SEQ ID NO: 2 or an antibody, which binds to the same epitope.

In one embodiment of the antibody for use in a method for the treatment of cancer in a subject, comprising, prior to the treatment of cancer in said subject, determining whether a target cell of said subject has SRRM2 present on the cell surface, said antibody is an antibody comprising a heavy chain variable region having an amino acid sequence with at least 99% identity to the amino acid sequence shown in SEQ ID NO: 1 and a light chain variable region having an amino acid sequence with at least 99% identity to the amino acid sequence shown in SEQ ID NO: 2 or an antibody, which binds to the same epitope.

In one embodiment of the antibody for use in a method for the treatment of cancer in a subject, comprising, prior to the treatment of cancer in said subject, determining whether a target cell of said subject has SRRM2 present on the cell surface, said antibody is an antibody comprising a heavy chain variable region having an amino acid sequence with at least 85% identity to the amino acid sequence shown in SEQ ID NO: 3 and a light chain variable region having an amino acid sequence with at least 85% identity to the amino acid sequence shown in SEQ ID NO: 4 or an antibody, which binds to the same epitope.

In one embodiment of the antibody for use in a method for the treatment of cancer in a subject, comprising, prior to the treatment of cancer in said subject, determining whether a target cell of said subject has SRRM2 present on the cell surface, said antibody is an antibody comprising a heavy chain variable region having an amino acid sequence with at least 90% identity to the amino acid sequence shown in SEQ ID NO: 3 and a light chain variable region having an amino acid sequence with at least 90% identity to the amino acid sequence shown in SEQ ID NO: 4 or an antibody, which binds to the same epitope.

In one embodiment of the antibody for use in a method for the treatment of cancer in a subject, comprising, prior to the treatment of cancer in said subject, determining whether a target cell of said subject has SRRM2 present on the cell surface, said antibody is an antibody comprising a heavy chain variable region having an amino acid sequence with at least 95% identity to the amino acid sequence shown in SEQ ID NO: 3 and a light chain variable region having an amino acid sequence with at least 95% identity to the amino acid sequence shown in SEQ ID NO: 4 or an antibody, which binds to the same epitope.

In one embodiment of the antibody for use in a method for the treatment of cancer in a subject, comprising, prior to the treatment of cancer in said subject, determining whether a target cell of said subject has SRRM2 present on the cell surface, said antibody is an antibody comprising a heavy chain variable region having an amino acid sequence with at least 99% identity to the amino acid sequence shown in SEQ ID NO: 3 and a light chain variable region having an amino acid sequence with at least 99% identity to the amino acid sequence shown in SEQ ID NO: 4 or an antibody, which binds to the same epitope.

In one embodiment of the antibody for use in a method for the treatment of cancer in a subject, comprising, prior to the treatment of cancer in said subject, determining whether a target cell of said subject has SRRM2 present on the cell surface, said antibody is an antibody comprising a heavy chain variable region having an amino acid sequence with at least 85% identity to the amino acid sequence shown in SEQ ID NO: 5 and a light chain variable region having an amino acid sequence with at least 85% identity to the amino acid sequence shown in SEQ ID NO: 6 or an antibody, which binds to the same epitope.

In one embodiment of the antibody for use in a method for the treatment of cancer in a subject, comprising, prior to the treatment of cancer in said subject, determining whether a target cell of said subject has SRRM2 present on the cell surface, said antibody is an antibody comprising a heavy chain variable region having an amino acid sequence with at least 90% identity to the amino acid sequence shown in SEQ ID NO: 5 and a light chain variable region having an amino acid sequence with at least 90% identity to the amino acid sequence shown in SEQ ID NO: 6 or an antibody, which binds to the same epitope.

In one embodiment of the antibody for use in a method for the treatment of cancer in a subject, comprising, prior to the treatment of cancer in said subject, determining whether a target cell of said subject has SRRM2 present on the cell surface, said antibody is an antibody comprising a heavy chain variable region having an amino acid sequence with at least 95% identity to the amino acid sequence shown in SEQ ID NO: 5 and a light chain variable region having an amino acid sequence with at least 95% identity to the amino acid sequence shown in SEQ ID NO: 6 or an antibody, which binds to the same epitope.

In one embodiment of the antibody for use in a method for the treatment of cancer in a subject, comprising, prior to the treatment of cancer in said subject, determining whether a target cell of said subject has SRRM2 present on the cell surface, said antibody is an antibody comprising a heavy chain variable region having an amino acid sequence with at least 99% identity to the amino acid sequence shown in SEQ ID NO: 5 and a light chain variable region having an amino acid sequence with at least 99% identity to the amino acid sequence shown in SEQ ID NO: 6 or an antibody, which binds to the same epitope.

In one embodiment of the antibody for use in a method for the treatment of cancer in a subject, comprising, prior to the treatment of cancer in said subject, determining whether a target cell of said subject has SRRM2 present on the cell surface, said antibody is an antibody comprising a heavy chain variable region having an amino acid sequence with at least 85% identity to the amino acid sequence shown in SEQ ID NO: 5 and a light chain variable region having an amino acid sequence with at least 85% identity to the amino acid sequence shown in SEQ ID NO: 26 or an antibody, which binds to the same epitope.

In one embodiment of the method of killing a target cell having serine/arginine repetitive matrix protein 2 (SRRM2) present on the cell surface of said target cell, said antibody is an antibody comprising a heavy chain variable region having an amino acid sequence with at least 90% identity to the amino acid sequence shown in SEQ ID NO: 5 and a light chain variable region having an amino acid sequence with at least 90% identity to the amino acid sequence shown in SEQ ID NO: 26 or an antibody, which binds to the same epitope.

In one embodiment of the antibody for use in a method for the treatment of cancer in a subject, comprising, prior to the treatment of cancer in said subject, determining whether a target cell of said subject has SRRM2 present on the cell surface, said antibody is an antibody comprising a heavy chain variable region having an amino acid sequence with at least 95% identity to the amino acid sequence shown in SEQ ID NO: 5 and a light chain variable region having an amino acid sequence with at least 95% identity to the amino acid sequence shown in SEQ ID NO: 26 or an antibody, which binds to the same epitope.

In one embodiment of the antibody for use in a method for the treatment of cancer in a subject, comprising, prior to the treatment of cancer in said subject, determining whether a target cell of said subject has SRRM2 present on the cell surface, said antibody is an antibody comprising a heavy chain variable region having an amino acid sequence with at least 99% identity to the amino acid sequence shown in SEQ ID NO: 5 and a light chain variable region having an amino acid sequence with at least 99% identity to the amino acid sequence shown in SEQ ID NO: 26 or an antibody, which binds to the same epitope.

In one embodiment of the antibody for use in a method for the treatment of cancer in a subject, comprising, prior to the treatment of cancer in said subject, determining whether a target cell of said subject has SRRM2 present on the cell surface, said antibody is an antibody comprising a heavy chain variable region comprising heavy chain CDR1 having the amino acid sequence as set forth in SEQ ID NO: 7, heavy chain CDR2 having the amino acid sequence as set forth in SEQ ID NO: 8, and heavy chain CDR3 having the amino acid sequence as set forth in SEQ ID NO: 9, and a light chain variable region comprising light chain CDR1 having the amino acid sequence as set forth in SEQ ID NO: 10, light chain CDR2 having the amino acid sequence as set forth in SEQ ID NO: 11, and light chain CDR3 having the amino acid sequence as set forth in SEQ ID NO: 12, or an antibody, which binds to the same epitope.

In one embodiment of the antibody for use in a method for the treatment of cancer in a subject, comprising, prior to the treatment of cancer in said subject, determining whether a target cell of said subject has SRRM2 present on the cell surface, said antibody is an antibody comprising a heavy chain variable region comprising heavy chain CDR1 having the amino acid sequence as set forth in SEQ ID NO: 13, heavy chain CDR2 having the amino acid sequence as set forth in SEQ ID NO: 14, and heavy chain CDR3 having the amino acid sequence as set forth in SEQ ID NO: 15, and a light chain variable region comprising light chain CDR1 having the amino acid sequence as set forth in SEQ ID NO: 16, light chain CDR2 having the amino acid sequence as set forth in SEQ ID NO: 17, and light chain CDR3 having the amino acid sequence as set forth in SEQ ID NO: 18, or an antibody, which binds to the same epitope.

In one embodiment of the antibody for use in a method for the treatment of cancer in a subject, comprising, prior to the treatment of cancer in said subject, determining whether a target cell of said subject has SRRM2 present on the cell surface, said antibody is an antibody comprising a heavy chain variable region comprising heavy chain CDR1 having the amino acid sequence as set forth in SEQ ID NO: 19, heavy chain CDR2 having the amino acid sequence as set forth in SEQ ID NO: 20, and heavy chain CDR3 having the amino acid sequence as set forth in SEQ ID NO: 21, and a light chain variable region comprising light chain CDR1 having the amino acid sequence as set forth in SEQ ID NO: 22, light chain CDR2 having the amino acid sequence as set forth in SEQ ID NO: 23, and light chain CDR3 having the amino acid sequence as set forth in SEQ ID NO: 24, or an antibody, which binds to the same epitope.

In one embodiment of the antibody for use in a method for the treatment of cancer in a subject, comprising, prior to the treatment of cancer in said subject, determining whether a target cell of said subject has SRRM2 present on the cell surface, said antibody is an antibody comprising a heavy chain variable region comprising heavy chain CDR1 having the amino acid sequence as set forth in SEQ ID NO: 19, heavy chain CDR2 having the amino acid sequence as set forth in SEQ ID NO: 20, and heavy chain CDR3 having the amino acid sequence as set forth in SEQ ID NO: 21, and a light chain variable region comprising light chain CDR1 having the amino acid sequence as set forth in SEQ ID NO: 27, light chain CDR2 having the amino acid sequence as set forth in SEQ ID NO: 28, and light chain CDR3 having the amino acid sequence as set forth in SEQ ID NO: 29, or an antibody, which binds to the same epitope.

All embodiments, which are described herein for the pharmaceutical composition of the present invention, the antibody, which binds to human SRRM2 present on the cell surface of a target cell of the present invention, the method of determining whether a human subject may suffer from cancer of the present invention as described herein as well as the respective definitions as described above, also apply for the antibody for use in a method for the treatment of cancer in a subject, comprising, prior to the treatment of cancer in said subject, determining whether a target cell of said subject has SRRM2 present on the cell surface as described herein.

The description of the sequences, shown in the sequence listing and as used in the context of the present invention, is as follows:
SEQ ID NO: 1 shows the amino acid sequence of the VH-region of antibody 13F11.
SEQ ID NO: 2 shows the amino acid sequence of the VL-region of antibody 13F11.
SEQ ID NO: 3 shows the amino acid sequence of the VH-region of antibody 23A7.
SEQ ID NO: 4 shows the amino acid sequence of the VL-region of antibody 23A7.
SEQ ID NO: 5 shows the amino acid sequence of the VH-region of antibody 18A4 and of antibody 18A4-2.
SEQ ID NO: 6 shows the amino acid sequence of the VL-region of antibody 18A4.
SEQ ID NO: 7 shows the amino acid sequence of the VH-CDR1 of antibody 13F11.
SEQ ID NO: 8 shows the amino acid sequence of the VH-CDR2 of antibody 13F11.
SEQ ID NO: 9 shows the amino acid sequence of the VH-CDR3 of antibody 13F11.
SEQ ID NO: 10 shows the amino acid sequence of the VL-CDR1 of antibody 13F11.
SEQ ID NO: 11 shows the amino acid sequence of the VL-CDR2 of antibody 13F11.
SEQ ID NO: 12 shows the amino acid sequence of the VL-CDR3 of antibody 13F11.
SEQ ID NO: 13 shows the amino acid sequence of the VH-CDR1 of antibody 23A7.
SEQ ID NO: 14 shows the amino acid sequence of the VH-CDR2 of antibody 23A7.
SEQ ID NO: 15 shows the amino acid sequence of the VH-CDR3 of antibody 23A7.
SEQ ID NO: 16 shows the amino acid sequence of the VL-CDR1 of antibody 23A7.
SEQ ID NO: 17 shows the amino acid sequence of the VL-CDR2 of antibody 23A7.
SEQ ID NO: 18 shows the amino acid sequence of the VH-CDR3 of antibody 23A7.
SEQ ID NO: 19 shows the amino acid sequence of the VH-CDR1 of antibody 18A4 and of antibody 18A4-2.
SEQ ID NO: 20 shows the amino acid sequence of the VH-CDR2 of antibody 18A4 and of antibody 18A4-2.
SEQ ID NO: 21 shows the amino acid sequence of the VH-CDR3 of antibody 18A4 and of antibody 18A4-2.
SEQ ID NO: 22 shows the amino acid sequence of the VL-CDR1 of antibody 18A4.
SEQ ID NO: 23 shows the amino acid sequence of the VL-CDR2 of antibody 18A4.
SEQ ID NO: 24 shows the amino acid sequence of the VL-CDR3 of antibody 18A4.
SEQ ID NO: 25 shows the amino acid sequence of SRRM2 as shown in UniProt database entry Q9UQ35, version 2 of 6 Mar. 2007.
SEQ ID NO: 26 shows the amino acid sequence of the VL-region of antibody 18A4-2.
SEQ ID NO: 27 shows the amino acid sequence of the VL-CDR1 of antibody 18A4-2.
SEQ ID NO: 28 shows the amino acid sequence of the VL-CDR2 of antibody 18A4-2.
SEQ ID NO: 29 shows the amino acid sequence of the VL-CDR2 of antibody 18A4-2.
SEQ ID NO: 30 shows the amino acid sequence of fragment tr04.

An overview of the SEQ ID NOs and detailed sequences, as used in the context of the present invention, is given in the following Table 1 (in case of conflict between the sequences shown in Table 1 and the sequences of the sequence listing, which has to be filed for formal reasons, the sequences of Table 1 supersede the sequences of the sequence listing):

TABLE 1

| SEQ | | |
|---|---|---|
| ID | | Sequence |
| NO: | Designation | (CDRs are underlined in the order CDR 1, 2, 3 starting from left upper line) |
| 1 | 13F11 VH | EVQLVESGGGLVQPGRSLKLSCAASRFTFSNYDMAWVRQAPTKGLEWVASISPSNVNTYYRDSVKGRF |
| | | TVSRDNAKSSLYLQMDSLRSEDTATYYCARLVHSFWYFDFWGPGTMVTVSS |
| 2 | 13F11 VL | DIVMTQTPSSQAVSAGEKVTMRCKSSQSLLYSKNKKNYLAWYQQKPGQSPKLLIYWASTRESGVPDRFL |
| | | GSGSGTDFTLTISSVQAEDLAVYYCQQYYKFPPTFGGGTKLELKRA |
| 3 | 23A7 VH | EVQLVESGGDLVQPGRSLKLSCAASGFTFSNYDMAWVRQAPTQGLEWVASISGSGGTTYYRDSVKGRF |
| | | TVSRDNAKSTLYLQMDGLRSEDTATYFCARHPPSFWYFDFWGPGSMVTVSS |
| 4 | 23A7 VL | DIVMTQTPSSQAVSAGEKVTMSCKSGQSLLFNKNKKDYLAWYQRKPGQSPKLLIYWASTRESGVPDRFI |
| | | GGGSGTDFTLTISSVQAEDLAVYYCQQYYRFPPTFGHGTKLELKRA |
| 5 | 18A4 VH and | EVQLVESGGGLVQPGRSLKLSCAASGFTFSNYDMAWVRQAPTKGLEWVASISPSGGSIYYRDSVKGRF |
| | 18A4-2 VH | TVSRNNAKSSLYLQMDSLRSEDTATYYCARLTTYFWYFDFWGPGTMVTVSS |
| 6 | 18A4 VL | DIQMTQSPSFLSASVGDRVTINCKASQNSNKYLNWYQQKLGEAPKLLIYNTNNLQTGIPSRFSGSGSGTD |
| | | YTLTISSLQPEDVATYFCLQHSSRMDTFGAGTKLELKRA |
| 7 | 13F11 VH-CDR1 | NYDMA |
| 8 | 13F11 VH-CDR2 | SISPSNVNTYYRDSVKG |
| 9 | 13F11 VH-CDR3 | LVHSFWYFDF |
| 10 | 13F11 VL-CDR1 | KSSQSLLYSKNKKNYLA |
| 11 | 13F11 VL-CDR2 | WASTRES |
| 12 | 13F11 VL-CDR3 | QQYYKFPPT |
| 13 | 23A7 VH-CDR1 | NYDMA |
| 14 | 23A7 VH-CDR2 | SISGSGGTTYYRDSVKG |
| 15 | 23A7 VH-CDR3 | HPPSFWYFDF |
| 16 | 23A7 VL-CDR1 | KSGQSLLFNKNKKDYLA |
| 17 | 23A7 VL-CDR2 | WASTRES |
| 18 | 23A7 VL-CDR3 | QQYYRFPPT |
| 19 | 18A4 VH-CDR1 | NYDMA |
| | and 18A4-2 VH- | |
| | CDR1 | |
| 20 | 18A4 VH-CDR2 | SISPSGGSIYYRDSVKG |
| | and 18A4-2 VH- | |
| | CDR2 | |
| 21 | 18A4 VH-CDR3 | LTTYFWYFDF |
| | and 18A4-2 VH- | |
| | CDR1 | |
| 22 | 18A4 VL-CDR1 | KASQNSNKYLN |
| 23 | 18A4 VL-CDR2 | NTNNLQT |
| 24 | 18A4 VL-CDR3 | LQHSSRMDT |
| 25 | SRRM2 | MYNGIGLPTPRGSGTNGYVQRNLSLVRGRRGERPDYKGEEELRRLEAALVKRPNPDILDHERKRRVELR |
| | | CLELEEMMEEQGYEEQQIQEKVATFRLMLLEKDVNPGGKEETPGQRPAVTETHQLAELNEKKNERLRAA |
| | | FGISDSYVDGSSFDPQRRAREAKQPAPEPPKPYSLVRESSSSRSPTPKQKKKKKKKDRGRRSESSSPR |
| | | RERKKSSKKKKHRSESESKKRKHRSPTPKSKRKSKDKKRKRSRSTTPAPKSRRAHRSTSADSASSSDT |
| | | SRSRSRSAAAKTHTTALAGRSPSPASGRRGEGDAPFSEPGTTSTQRPSSPETATKQPSSPYEDKDKDK |
| | | KEKSATRPSPSPERSSTGPEPPAPTPLLAERHGGSPQPLATTPLSQEPVNPPSEASPTRDRSPPKSPEK |
| | | LPQSSSSESSPPSPQPTKVSRHASSSPESPKPAPAPGSHREISSSPTSKNRSHGRAKRDKSHSHTPSRR |
| | | MGRSRSPATAKRGRSRSRTPTKRGHSRSRSPQWRRSRSAQRWGRSRSPQRRGRSRSPQRPGWSRS |
| | | RNTQRRGRSRSARRGRSHSRSPATRGRSRSRTPARRGRSRSRTPARRRSRSRTPTRRRSRSRTPARR |
| | | GRSRSRTPARRRSRTRSPVRRRSRSRSPARRSGRSRSRTPARRGRSRSRTPARRGRSRSRTPARRSG |
| | | RSRSRTPARRGRSRSRTPRRGRSRSRSLVRRGRSHSRTPQRRGRSGSSSERKNKSRTSQRRSRSNSS |
| | | PEMKKSRISSRRSRSLSSPRSKAKSRLSLRRSLSGSSPCPKQKSQTPPRRSRSGSSQPKAKSRTPPRRS |
| | | RSSSSPPPKQKSKTPSRQSHSSSSPHPKVKSGTPPRQGSITSPQANEQSVTPQRRSCFESSPDPELKS |
| | | RTPSRHSCSGSSPPRVKSSTPPRQSPSRSSSPQPKVKAIISPRQRSHSGSSSPSPSRVTSRTTPRRSRS |
| | | VSPCSNVESRLLPRYSHSGSSSPDTKVKPETPPRQSHSGSISPYPKVKAQTPPGPSLSGSKSPCPQEKS |
| | | KDSLVQSCPGSLSLCAGVKSSTPPGESYFGVSSLQLKGQSQTSPDHRSDTSSPEVRQSHSESPSLQSK |
| | | SQTSPKGGRSRSSSPVTELASRSPIRQDRGEFSASPMLKSGMSPEQSRFQSDSSSYPTVDSNSLLGQS |
| | | RLETAESKEKMALPPQEDATASPPRQKDKFSPFPVQDRPESSLVFKDTLRTPPRERSGAGSSPETKEQN |
| | | SALPTSSQDEELMEVVEKSEEPAGQILSHLSSELKEMSTSNFESSPEVEERPAVSLTLDQSQSQASLEAV |
| | | EVPSMASSWGGPHFSPEHKELSNSPLRENSFGSPLEFRNSGPLGTEMNTGFSSEVKEDLNGPFLNQLE |
| | | TDPSLDMKEQSTRSSGHSSSELSPDAVEKAGMSSNQSISSPVLDAVPRTPSRERSSSASSPEMKDGLP |
| | | RTPSRRSRSGSSPGLRDGSGTPSRHSLSGSSPGMKDIPRTPSRGRSECDSSPEPKALPQTPRPRSRSP |
| | | SSPELNNKCLTPQRERSGSESSVDQKTVARTPLGQRSRSGSSQELDVKPSASPQERSESDSSPDSKAK |
| | | TRTPLRQRSRSGSSPEVDSKSRLSPRRSRSGSSPEVKDKPRAAPRAQSGSDSSPEPKAPAPRALPRRS |
| | | RSGSSSKGRGPSPEGSSSTESSPEHPPKSRTARRGSRSSPEPKTKSRTPPRRRSSRSSPELTRKARLS |
| | | RRSRSASSSPETRSRTPPRHRRSPSVSSPEPAEKSRSSRRRRSASSPRTKTTSRRGRSPSPKPRGLQR |
| | | SRSRSRREKTRTTRRRDRSGSSQSTSRRRQRSRSRSRVTRRRRGGSGYHSRSPARQESSRTSSRRR |
| | | RGRSRTPPTSRKRSRSRTSPAPWKRSRSRASPATHRRSRSRTPLISRRRSRSRTSPVSRRRSRSRTSV |
| | | TRRRSRSRASPVSRRRSRSRTPPVTRRRSRSRTPTTRRRSRSRTPPVTRRRSRSRTPPVTRRRSRSRT |
| | | SPITRRRSRSRTSPVTRRRSRSRTSPVTRRRSRSRTSPVTRRRSRSRTPPAIRRRSRSRTPLLPRKRSR |
| | | SRSPLAIRRRSRSRTPRTARGKRSLTRSPPAIRRRSASGSSSDRSRSATPPATRNHSGSRTPPVALNSS |
| | | RMSCFSRPSMSPTPLDRCRSPGMLEPLGSSRTPMSVLQQAGGSMMDGPGPRIPDHQRTSVPENHAQS |
| | | RIALALTAISLGTARPPPSMSAAGLAARMSQVPAPVPLMSLRTAPAANLASRIPAASAAAMNLASARTPAI |
| | | PTAVNLADSRTPAAAAAMNLASPRTAVAPSAVNLADPRTPTAPAVNLAGARTPAALAALSLTGSGTPPTA |
| | | ANYPSSSRTPQAPASANLVGPRSAHATAPVNIAGSRTAAALAPASLTSARMAPALSGANLTSPRVPLSAY |
| | | ERVSGRTSPPLLDRARSRTPPSAPSQSRMTSERAPSPSSRMGQAPSQSLLPPAQDQPRSPVPSAFSDQ |
| | | SRCLIAQTTPVAGSQSLSSGAVATTTSSAGDHNGMLSVPAPGVPHSDVGEPPASTGAQQPSALAALQPA |
| | | KERRSSSSSSSSSSSSSSSSSSSSSSSSSGSSSSDSEGSSLPVQPEVALKRVPSPTPAPKEAVREGRPP |
| | | EPTPAKRKRRSSSSSSSSSSSSSSSSSSSSSSSSSSSSSSSSSSSSSSSSSSPSPAKPGPQALPKPASP |
| | | KKPPPGERRSRSPRKPIDSLRDSRSLSYSPVERRRPSPQPSPRDQQSSSSERGSRRGQRGDSRSPSH |
| | | KRRRETPSPRPMRHRSSRSP |
| 26 | 18A4-2 VL | DIVMTQTPSSQAVSAGEKVTMSCKSSQSLLYSENKKNYLAWYQQKPGQSPKLLIYWASTRESGVPDRFI |
| | | GSGSGTDFTLTISSVQAEDLAVYYCQQYYKFPPTFGSGTKLEIK |
| 27 | 18A4-2 VL-CDR1 | KSSQSLLYSENKKNYLA |
| 28 | 18A4-2 VL-CDR2 | WASTRES |
| 29 | 18A4-2 VL-CDR3 | QQYYKFPPT |
| 30 | fragment tr04 | RSRTSPVSRRRSRSRTSVTRRRSRSRASPVSRRRSRSRTPPVTRRRSRSRTPTTRRRSRSRTPPVTRR |
| | | RSRSRTPPVTRRRSRSRTSPITRRRSRSRTSPVTRRRSRSRTSPVTRRRSRSRTSPVTRRRSRSRTPPA |
| | | IRRRSRSRTPLLPRKRSRSRSPLAIRRRSRSRTPRTARGKRSLTRSPPAIRRRSASGSSSDRSRSATPPA |
| | | TRNHSGSRTPPVALNSSRMSCFSRPSMSPTPLDRCRSPGMLEPLGSSRTPMSVLQ |

The following abbreviations were used in the context of the present invention: VH=variable heavy chain; VH=variable heavy chain; VL=variable light chain; CDR=complementarity determining region; VH-CDR=CDR of a variable heavy region; VL-CDR=CDR of a variable light region; CDRs can be determined by using the Kabat algorithm, e.g., http://abysis.org/abysis/.

It is noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or”, wherever used herein, includes the meaning of “and”, “or” and “all or any other combination of the elements connected by said term”.

The term “less than” or in turn “more than” does not include the concrete number.

For example, “less than 20” means less than the number indicated. Similarly, “more than” or “greater than” means more than or greater than the indicated number, e.g. “more than 80%” means more than or greater than the indicated number of 80%.

Throughout this specification and the claims which follow, unless the context requires otherwise, the word “comprise”, and variations such as “comprises” and “comprising”, will be understood to imply the inclusion of a stated integer or step or group of integers or steps, but not the exclusion of any other integer or step or group of integer or step. When used herein the term “comprising” can be substituted with the term “containing” or “including” or sometimes, when used herein, with the term “having”. When used herein, “consisting of” excludes any element, step, or ingredient not specified.

The term “including” means “including but not limited to”. “Including” and “including but not limited to” are used interchangeably.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

All publications cited throughout the text of this specification (including all patents, patent application, scientific publications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

The content of all documents and patent documents cited herein is incorporated by reference in their entirety.

A better understanding of the present invention and of its advantages will be had from the following examples, offered for illustrative purposes only. The examples are not intended to limit the scope of the present invention in any way.

The invention is further characterized by the following items:

1. Pharmaceutical composition comprising an antibody, preferably a monoclonal antibody, which binds to human Serine/arginine repetitive matrix protein 2 (SRRM2) present on the cell surface of a target cell and optionally a pharmaceutically acceptable carrier, diluent or excipient.
2. The pharmaceutical composition of item 1, wherein said antibody has cytotoxic activity.
3. The pharmaceutical composition of any one of the preceding items, wherein said antibody has ADCC or CDC.
4. The pharmaceutical composition of any one of the preceding items, wherein said antibody is conjugated with a cytotoxic substance.
5. The pharmaceutical composition of any one of the preceding items, wherein said antibody is part of a chimeric antigen receptor (CAR).
6. The pharmaceutical composition of item 5, wherein said CAR is comprised by a T cell, NK cell, NK-T cell or macrophage.
7. The pharmaceutical composition of any one of the preceding items, wherein said SRRM2 present on the cell surface of a target cell is externalized.
8. The pharmaceutical composition of any one of the preceding items, wherein the antibody is not an intracellular antibody.
9. The pharmaceutical composition of any one of the preceding items, wherein said antibody is
(a) an antibody comprising a heavy chain variable region having an amino acid sequence with at least 85% identity to the amino acid sequence shown in SEQ ID NO: 1 and a light chain variable region having an amino acid sequence with at least 85% identity to the amino acid sequence shown in SEQ ID NO: 2;
(b) an antibody comprising a heavy chain variable region having an amino acid sequence with at least 85% identity to the amino acid sequence shown in SEQ ID NO: 3 and a light chain variable region having an amino acid sequence with at least 85% identity to the amino acid sequence shown in SEQ ID NO: 4;
(c) an antibody comprising a heavy chain variable region having an amino acid sequence with at least 85% identity to the amino acid sequence shown in SEQ ID NO: 5 and a light chain variable region having an amino acid sequence with at least 85% identity to the amino acid sequence shown in SEQ ID NO: 6;
(d) an antibody which binds to the same epitope as that in the human SRRM2 protein to which any one of the antibodies (a) to (c) bind.
   10. The pharmaceutical composition of any one of the preceding items, wherein said antibody is
(a) an antibody comprising a heavy chain variable region comprising heavy chain CDR1 having the amino acid sequence as set forth in SEQ ID NO: 7, heavy chain CDR2 having the amino acid sequence as set forth in SEQ ID NO: 8, and heavy chain CDR3 having the amino acid sequence as set forth in SEQ ID NO: 9, and
a light chain variable region comprising light chain CDR1 having the amino acid sequence as set forth in SEQ ID NO: 10, light chain CDR2 having the amino acid sequence as set forth in SEQ ID NO: 11, and light chain CDR3 having the amino acid sequence as set forth in SEQ ID NO: 12;
(b) an antibody comprising a heavy chain variable region comprising heavy chain CDR1 having the amino acid sequence as set forth in SEQ ID NO: 13, heavy chain CDR2 having the amino acid sequence as set forth in SEQ ID NO: 14, and heavy chain CDR3 having the amino acid sequence as set forth in SEQ ID NO: 15, and
a light chain variable region comprising light chain CDR1 having the amino acid sequence as set forth in SEQ ID NO: 16, light chain CDR2 having the amino acid sequence as set forth in SEQ ID NO: 17, and light chain CDR3 having the amino acid sequence as set forth in SEQ ID NO: 18;
(c) an antibody comprising a heavy chain variable region comprising heavy chain CDR1 having the amino acid sequence as set forth in SEQ ID NO: 19, heavy chain CDR2 having the amino acid sequence as set forth in SEQ ID NO: 20, and heavy chain CDR3 having the amino acid sequence as set forth in SEQ ID NO: 21, and
a light chain variable region comprising light chain CDR1 having the amino acid sequence as set forth in SEQ ID NO: 22, light chain CDR2 having the amino acid sequence as set forth in SEQ ID NO: 23, and light chain CDR3 having the amino acid sequence as set forth in SEQ ID NO: 24;
(d) an antibody which binds to the same epitope as that in the human SRRM2 protein to which any one of the antibodies (a) to (c) bind.
   11. The pharmaceutical composition of any one of the preceding items for use in a method for the treatment of cancer in a human subject, preferably wherein said cancer is characterized by cells, wherein human SRRM2 is present on their cell surface.
   12. The pharmaceutical composition for the use of item 11, wherein said cancer is breast cancer, prostate cancer, lung cancer, colorectal cancer, ovarian cancer, pancreatic cancer, stomach cancer, cholangiogenic cancer, oral cavity squamous carcinoma, hematologic malignancy tumor, or glioma.
   13. A method for determining whether a human subject may suffer from cancer, comprising determining in a sample obtained from said human subject whether SRRM2 is present on the cell surface of cells comprised by said sample and/or whether SRRM2 is present on the surface of extracellular vesicles.
   14. An antibody which binds to human SRRM2 present on the cell surface of a target cell, wherein said antibody is
(a) an antibody comprising a heavy chain variable region having an amino acid sequence with at least 85% identity to the amino acid sequence shown in SEQ ID NO: 1 and a light chain variable region having an amino acid sequence with at least 85% identity to the amino acid sequence shown in SEQ ID NO: 2;
(b) an antibody comprising a heavy chain variable region having an amino acid sequence with at least 85% identity to the amino acid sequence shown in SEQ ID NO: 3 and a light chain variable region having an amino acid sequence with at least 85% identity to the amino acid sequence shown in SEQ ID NO: 4;
(c) an antibody comprising a heavy chain variable region having an amino acid sequence with at least 85% identity to the amino acid sequence shown in SEQ ID NO: 5 and a light chain variable region having an amino acid sequence with at least 85% identity to the amino acid sequence shown in SEQ ID NO: 6;
(d) an antibody which binds to the same epitope as that in the human SRRM2 protein to which any one of the antibodies (a) to (c) bind.
   15. An antibody which binds to human SRRM2 present on the cell surface of a target cell comprising
(a) an antibody comprising a heavy chain variable region comprising heavy chain CDR1 having the amino acid sequence as set forth in SEQ ID NO: 7, heavy chain CDR2 having the amino acid sequence as set forth in SEQ ID NO: 8, and heavy chain CDR3 having the amino acid sequence as set forth in SEQ ID NO: 9, and
a light chain variable region comprising light chain CDR1 having the amino acid sequence as set forth in SEQ ID NO: 10, light chain CDR2 having the amino acid sequence as set forth in SEQ ID NO: 11, and light chain CDR3 having the amino acid sequence as set forth in SEQ ID NO: 12;
(b) an antibody comprising a heavy chain variable region comprising heavy chain CDR1 having the amino acid sequence as set forth in SEQ ID NO: 13, heavy chain CDR2 having the amino acid sequence as set forth in SEQ ID NO: 14, and heavy chain CDR3 having the amino acid sequence as set forth in SEQ ID NO: 15, and
a light chain variable region comprising light chain CDR1 having the amino acid sequence as set forth in SEQ ID NO: 16, light chain CDR2 having the amino acid sequence as set forth in SEQ ID NO: 17, and light chain CDR3 having the amino acid sequence as set forth in SEQ ID NO: 18;
(c) an antibody comprising a heavy chain variable region comprising heavy chain CDR1 having the amino acid sequence as set forth in SEQ ID NO: 19, heavy chain CDR2 having the amino acid sequence as set forth in SEQ ID NO: 20, and heavy chain CDR3 having the amino acid sequence as set forth in SEQ ID NO: 21, and
a light chain variable region comprising light chain CDR1 having the amino acid sequence as set forth in SEQ ID NO: 22, light chain CDR2 having the amino acid sequence as set forth in SEQ ID NO: 23, and light chain CDR3 having the amino acid sequence as set forth in SEQ ID NO: 24;
(d) an antibody which binds to the same epitope as that in the human SRRM2 protein to which any one of the antibodies (a) to (c) bind.

### EXAMPLES

### Materials and Methods

### Cells and Culture Conditions

Cells were cultured in DMEM/F12 medium (Gibco BRL, Karlsruhe, Germany), supplemented with 8% v/v fetal bovine serum, 1% v/v L-Glutamine and 1% v/v penicillin-streptomycin). All cells were incubated in a humidified CO₂ incubator at 37° C., 18.3% O₂ and 5% CO₂. Hypoxic cells were incubated at 1% O₂.

### Transfection of 293 Cells

293 cells were transfected with expression plasmids, coding for human SRRM2 or mutants thereof using Lipofectamine™ 2000 transfection reagent (ThermoFisher Scientific, Menzel, Germany) according to manufacturer's instructions. Where appropriate, cells were selected for positive transfected cells with 80 μg/ml Hygromycin C and further cultured in normal growth medium supplemented with 80 μg/ml Hygromycin C.

Isolation of Primary Peripheral Blood Mononuclear Cells (PBMCs) from Fresh Blood

30 ml fresh heparinized blood from healthy donors were diluted with PBS and loaded carefully on top of a layer of 10 ml human Pancoll (Pan Biotech, Aidenbach, Germany). The tubes were centrifuged for 25 min at 800×g. After that, PBMCs were carefully collected from the intermediate layer between Pancoll and plasma and washed three times in PBS. Cells were used fresh for experiments or immediately frozen and stored at −80° C. for later experiments.

### Flow Cytometry

For flow cytometric analysis of SRRM2 expression, cells were stained with either of the three SRRM2-specific antibodies in FACS-Buffer (PBS+2% FSC) or with an isotype control antibody for 20 min followed by staining with an anti-rat-Alexa Fluor® 647 secondary antibody (Jackson Immuno Research). All stainings were performed on ice.

### Immunoblotting

For analysis of SRRM2 expression by western blotting, whole cell lysates were prepared. Adherent cells were washed with PBS, detached by trypsin and pelleted. The cell pellet was washed in PBS, after that cells were resuspended in RIPA buffer with protease inhibitors (Roche Diagnostics) and incubated on ice for 20 min. Cell fragments were centrifuged for 20 min at 4° C. at max speed. The supernatant was transferred to a new tube and the protein content was determined by Bradford assay (Bio-Rad). 20 μg of cell lysate were used for SDS-PAGE. Gels were electroblotted on a nitrocellulose membrane and blocked with 5% milk in TBST. Following primary antibodies were used for Western blotting: anti-SRRM2 (Thermo Scientific), anti-GFP (kind gift of the core facility ‘monoclonal antibodies’, Helmholtz Center Munich). All primary antibodies were incubated at 4° C. over night. The membrane was washed and incubated with HRP-conjugated secondary antibodies at room temperature for 2 h. The following secondary antibodies were used: anti-rat IgG-HRP (Cell Signaling), anti-mouse IgG-HRP (Cell Signaling Technology, Frankfurt a. Main, Germany) and anti-rabbit IgG-HRP (Cell Signaling). Detection of proteins was performed with the ECL system (GE Healthcare).

### Immunoprecipitation

Immunoprecipitation was performed using CNBr beads (CNBr-activated Sepharose 4 Fast Flow, GE Healthcare). 1 g beads were solved in 10 ml 1 mM HCl and incubated at room temperature for 20 min. After that beads were spin down for 1 min at 3000×g and washed 15×. Then, a subclass specific mouse anti-rat IgG2a antibody was coupled to the beads (2 mg antibody in coupling buffer 0.3 M NaHCO₃, 1.5 M NaCl, pH 8.3) for 1 h at room temperature. After that, beads were washed in coupling buffer and all remaining binding sites blocked with 1 M ethanolamine (1 M) for 2 h at room temperature. After washing in wash buffer (0.1 M Tris/HCl, 0.5 M NaCl, pH 4) and in NaOAc buffer (0.1 M NaOAc, 0.5 M NaCl) coupled beads were resuspended in PBS and used for coupling with 22E6. Therefore, 500 μl 22E6 hybridoma supernatant was incubated with 60 μl anti-subclass specific beads over night at 4° C.

Beads were then washed in PBS and incubated with 2 mg cell lysate or 0.75 μg sCD73 (human CD73/NT5E protein, his tagged, Sino Biologicals) at 4° C. over night. After that, beads were washed in RIPA buffer with protease inhibitors, PBS+ (PBS, 0.5% N-laurylsarcosine, 0.1% SDS) and in PBS. The supernatant was discarded, beads resuspended in 3× Laemmli buffer and incubated at room temperature for 3 min. After centrifugation for 5 min at 1,000×g the supernatant was used for western blot analysis.

### Generation of SRRM2 Knockout Cells

SRRM2-specific CRISPR-RNAs (crRNA) were designed with a freely available online tool (IDT). Each of those different specific crRNAs were fused to a transactivating RNA (tracrRNA) building a guideRNA (gRNA), which was then complexed with CAS9 Nuclease. Individual Complex-combinations were introduced into A549 lung cancer cells by electroporation using a 4D-Nucleofector Protocol from Lonza. Electroporated cells were analyzed by Fluorescence Activated Cell Sorting (FACS) one week later for binding of SRRM2 antibodies 13F11, 18A4 and 23A7. Cell clones were generated by sorting and limited dilution cloning. Finally, clones were re-analyzed by FACS. Identified knockout clones were then checked by screening for CRISPR/CAS9 mediated mutation. Therefore, genomic DNA from parental cells and knockout clones were isolated, sequenced and compared to the wildtype sequence.

### Confocal Microscopy

1×10⁵ cells were seeded on cover slips and grown over night. The medium was removed and cells were washed with PBS, fixed in 4% PFA for 10 min, permeabilized with PBS containing 0.3% Tween20 and finally blocked with 3% BSA in TBS with 0.1% Tween 20. For staining, cells were incubated with the 1^{st} antibody, diluted in TBS with 1% BSA and 0.1% Tween 20 for 1 hour at room temperature. Then, cells were washed three times before incubation with the secondary antibody (goat anti-rat-Alexa 647) in the dark for 40 min. After a final washing step, nuclei were stained with DAPI, a mounting medium (Vectashield) was added and cover slips were mounted upside-down on a microscope slides. Slides were analyzed on a Leica SP8X STED microscope using the Leica LAS X software.

### Immunohistochemistry

Paraffin sections were mounted onto superform plus slides. After fixation in acetone, slides were incubated with the primary antibody followed by an incubation with a suitable biotinylated secondary antibody. Slides were developed with 0.01% 3-amino-9-ethylcarbazole (AEC) as chromogen. After counterstaining with hematoxylin, slides were cover-slipped with Kaiser's glycerol gelatine.

### Results

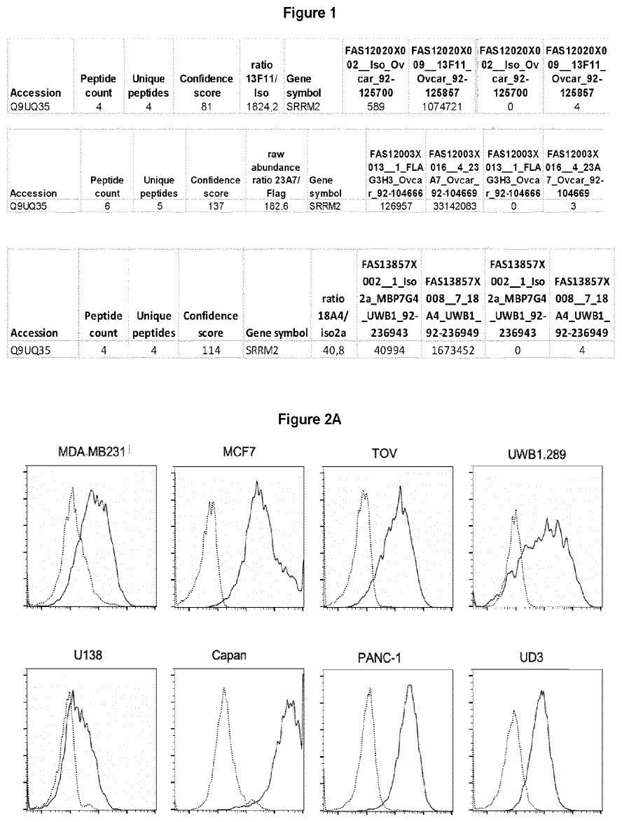

Example 1 shows that the monoclonal antibodies 13F11, 18A4 and 23A7 specifically precipitate SRRM2 from the lysates of various cancer cell lines. Any of the antibody was coupled to activated cyanogen bromide beads and incubated with lysate of ovarian cancer cell lines at 4° C. overnight. The next day, beads were precipitated by centrifugation, the bound proteins were eluted in Laemmli buffer and analyzed by mass spectrometry. Three independent experiments revealed confidence scores of >60, and enrichments of precipitated SRRM2 by factors of 1824.2, 182.6 and 40.8 as compared to precipitation with an isotype control antibody (see FIG. 1).

Example 2 shows that SRRM2 is exposed on the surface of various cancer cell lines. Cells were incubated with 13F11 (solid lines in FIG. 2A and FIG. 2B) or an isotype control antibody (dotted line in FIG. 2A, tinted histogram in FIG. 2B). Cells were then washed and incubated with an Alexa647 labeled anti-rat IgG antibody. Fluorescence was then measured by flow cytometry on a FACS Canto. Normal PBMCs do not express surface SRRM2.

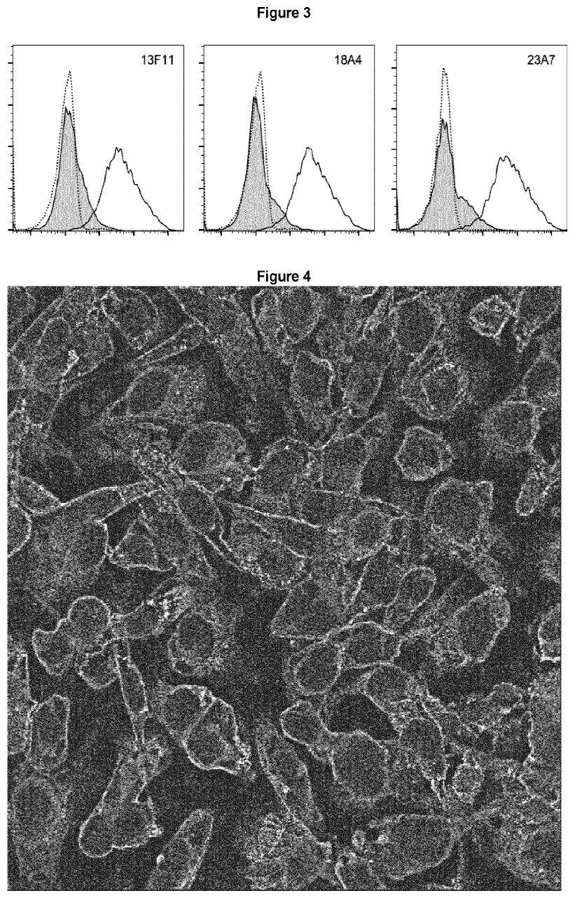

Example 3 shows that 13F11, 23A7 and 18A4 are SRRM2-specific: A549 human lung cancer cells were transfected with a ribonucleoprotein complex consisting of SRRM2-specific gRNAs and Cas9 protein. Seven days later, binding of the antibodies to knockout and wildtype cells was compared by flow cytometry. For this, A549 Wildtype (bold line) and the A549 SRRM Knockout line #25 (tinted histogram) were incubated with any of the three antibodies 13F11, 18A4 and 23A7. Cells were washed and then incubated with an Alexa647 labeled anti-rat secondary antibody. Fluorescence was then measured on a FACS Canto. An isotype control antibody was included as a negative control (dotted line). Reduced fluorescence intensity of line #25 indicates the specificities of the antibodies for SRRM2 (see FIG. 3).

Example 4 further illustrates the membrane localization of SRRM2 on human cancer cells. SK-OV-3 human ovarian cancer cells were grown on microscope slides, washed in PBS, and incubated with the antibody 13F11. Incubation with the antibody prior to fixation secures that cells were vital and thus that the antibody bound to intact cells. Then, cells were fixed with 4% paraformaldehyde and again incubated with an Alexa647-coupled suitable anti-rat IgG secondary antibody. The staining was then inspected by confocal fluorescence microscopy (see FIG. 4).

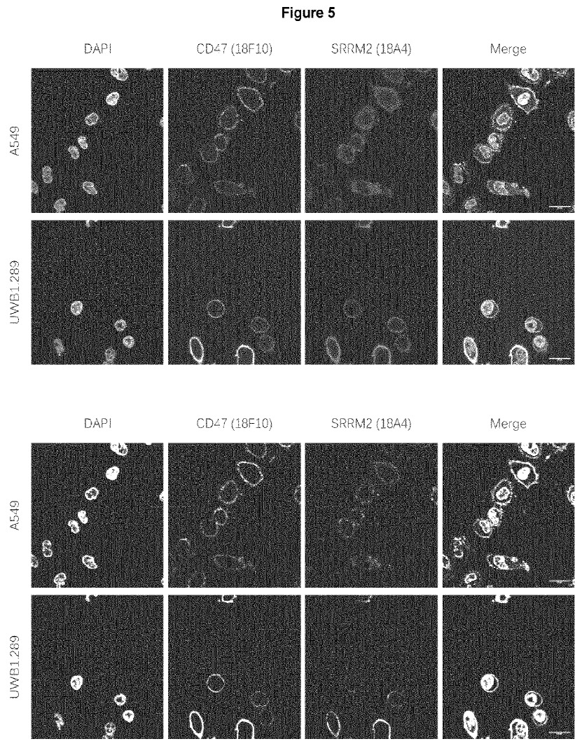

Example 5 even further illustrates the membrane localization of SRRM2 on human cancer cells. Here, UWB1.289 human ovarian cancer cells were co-stained with the SRRM2-specific antibody 13F11 (magenta) and antibody 18F10, specific for the membrane protein CD47 (green). After fixation as described above, cells were analyzed by confocal fluorescence microscopy. Nuclei were counter-stained with DAPI (blue). The white color in the merged pictures indicates co-localization of both antibodies (see FIG. 5).

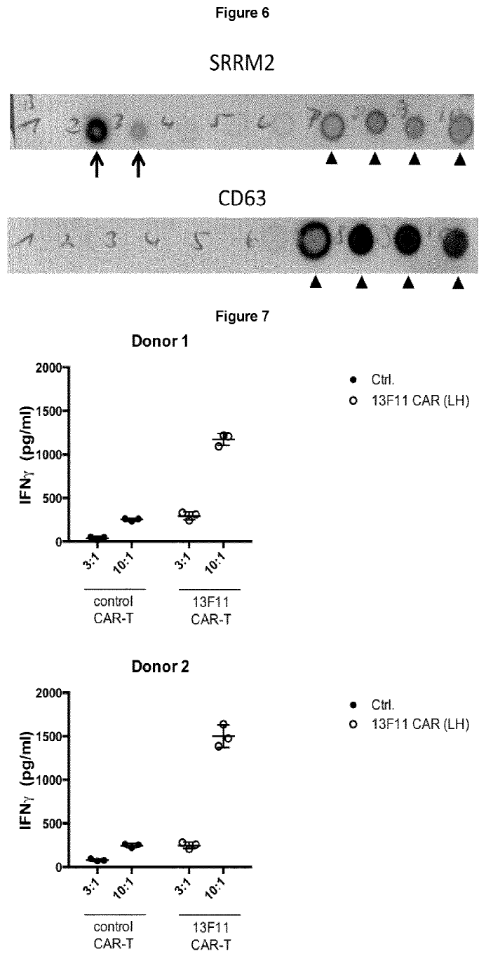

Example 6 shows that SRRM2 is localized to lipid rafts of other membrane sub-domains resistant to lysis with RIPA buffer. SK-OV-3 cells were lysed in RIPA buffer (150 mM NaCl, 50 mM Tris/HCl (pH 7.4), 1% Igepal, 0.5% Natriumdesoxycholat). The lysate was then loaded onto a Optiprep gradient and centrifuged at 32.000 rpm overnight. Fractions of 400 μl were taken and 2 μl of each fraction was spotted onto a nitrocellulose filter. These filters were then incubated with 13F11 or an antibody specific for the tetraspanin CD63. As can been seen in FIG. 6, SRRM2 is present in fractions 2 and 3 of the gradient, corresponding to a density of approx. 1.13-1.19 g/ml and thus to the supposed density of lipid rafts and other insoluble membrane microdomains. Soluble proteins, which have a higher specific density, are mainly localized to fractions 7-10 of the gradient.

Example 7 shows that surface-SRRM2-positive SK-OV-3-cells activate SRRM2-specific 13F11 CAR-T-cells. Primary T cells from two donors (1 and 2) were transduced with a retroviral vector encoding a chimeric antigen receptor (CAR) with a single-chain construct derived from antibody 13F11. Expression of the CAR on transduced T cells was tested by flow cytometry with a goat-anti rat IgG antibody (not shown). SK-OV-3 cells were incubated for 24 h with 13F11 CAR-T cells (columns 3-4 of the blots) of non-transduced control T cells (column 1-2 of the blots) at the E:T ratios indicated on the X-axis. Finally, interferon-gamma in the supernatants, which is a marker of T-cell activation, was quantified with a commercial ELISA assay (see FIG. 7).

Example 8 shows that the SRRM2-specific CAR-T cells also kill SRRM2-positive HO-8910 target cells. Here, killing was measured with a commercial LDH assay (see FIG. 8).

Example 9 shows that extracellular vesicles (EVs) isolated from ascites of a patient with ovarian cancer are SRRM2-positive. EVs were isolated by ultracentrifugation and density gradient centrifugation. A sample of the EVs was spotted onto a nitrocellulose membrane and incubated with 13F11, 18A4 or 23A7, followed by incubation with a HRP-coupled anti-rat secondary antibody. Membranes were developed with ECL (see FIG. 9). As a control, an irrelevant isotype antibody was used (rightmost image 4 in FIG. 9).

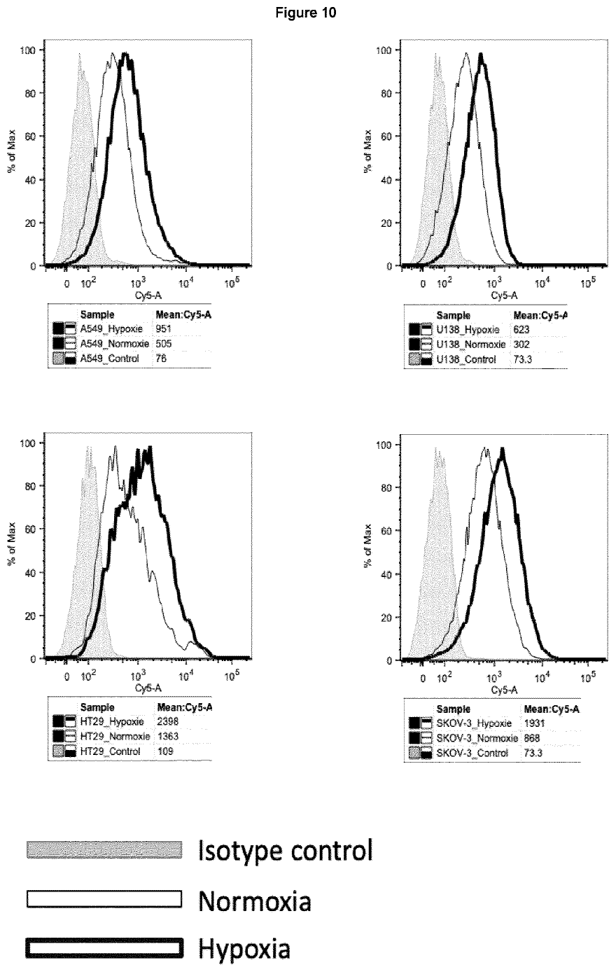

Example 10 shows that surface SRRM2 on cancer cells is induced by hypoxia. Most marketed therapeutic antibodies for the treatment of solid cancer are targeting hypoxia-induced proteins. Because hypoxia is a common hallmark of solid tumors, these proteins are highly expressed on cancer cells. Also, induction of translation in hypoxia can be considered a hint towards the relevance of such proteins for the survival and growth of cancer cells under hypoxic conditions. To investigate whether SRRM2 is induced in hypoxia, the inventors cultivated human A549, U138, HT29 and SK-OV-3 cells under normoxic conditions (approx. 18% O₂) or under hypoxia (1% O₂) in standard DMEM medium for 72 h. Thereafter, cells were stained with the SRRM2-specific antibody 13F11 or an isotype control antibody. Binding of 13F11 was then quantified by flow cytometry and expressed as mean fluorescence intensity (‘Mean: Cy5-A’ in FIG. 10). Mean values of hypoxic cells are approximately twice as high as under normoxia, showing that surface SRRM2 is a hypoxia-induced protein (see FIG. 10).

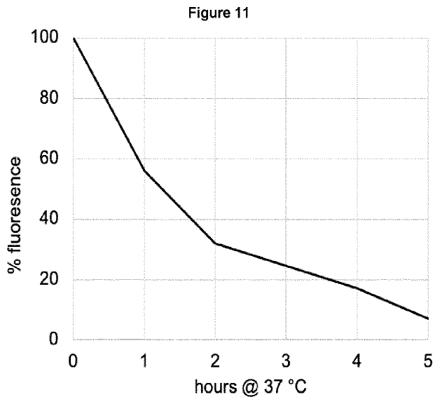

Example 11 shows 11F11 is internalized. Monoclonal antibodies can be modified to carry a toxin or radioactivity. These ‘antibody drug conjugates’ (ADCs) are increasingly used as a therapeutic regimen. For ADCs it is important that they bind to target cells and to be internalized into the target cells were the payloads can exert their activity. We tested the internalization of 13F11 by SK-OV-3 cells. For this, SK-OV-3 cells were incubated with 13F11 for 10 min at room temperature and then thoroughly washed to remove free antibody. Samples of these cells were then incubated at 37° C. for one to five hours or kept on ice (=0 hour). Then, the cells were stained with an Alexa647-coupled secondary anti-rat IgG antibody and fluorescence was measured by flow cytometry. An decrease in fluorescence over time indicates an internalization of the SRRM2-13F11 complex, resulting in reduced binding of the secondary antibody. The fluorescence measured after 0 hours at 37° C. was set to 100% (see FIG. 11).

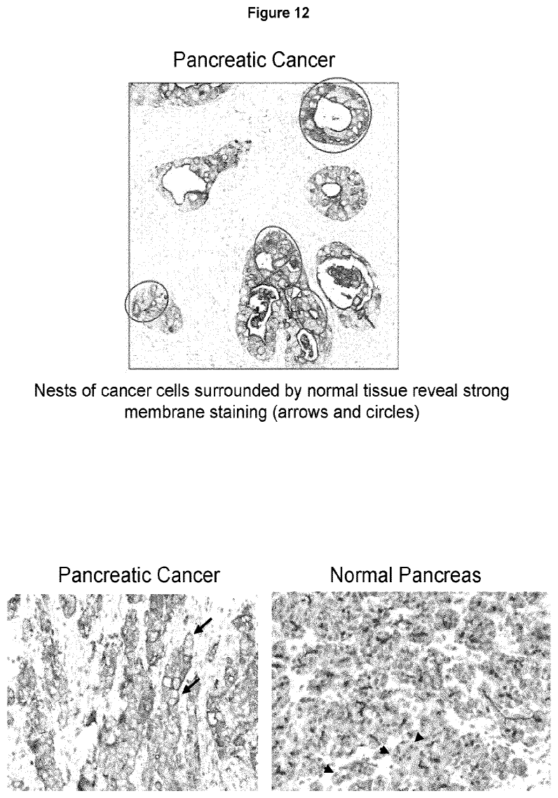

Example 12 shows that SRRM2 is expressed on the surface of primary pancreatic cancer. Paraffine sections derived from specimens of pancreatic cancer or normal pancreas were stained with 18A4, followed by a HRP-coupled secondary antibody and developed with ABC. As shown in FIG. 12, cancer cells clearly reveal membrane localization of SRRM2 (arrows) while in normal cells, the protein shows a nuclear staining pattern (arrowheads).

Example 13 shows that SRRM2 is expressed on the surface of lung metastases from human HUCT-1 cells xenotransplanted into immunocompromised SCID mice. In contrast, normal mouse tissues (brain, kidney, liver) reveal nuclear staining pattern.

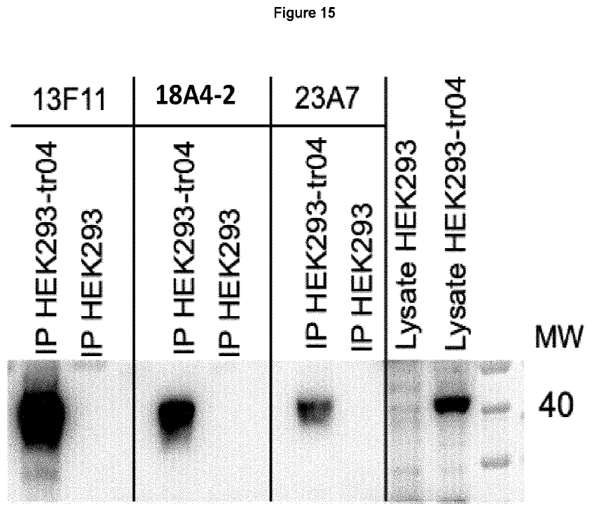

Example 14 shows that 13F11, 18A4-2 and 23A7 binds to a fragment (called herein tr04, SEQ ID NO: 30) spanning amino acids 1889 to 2150 of SRRM2 (see FIG. 15). HEK293 cells were transfected with an expression plasmid encoding amino acids 1889 to 2150 of SRRM2 fused to a HIS-tag (HEK293-tr04). Immunoprecipitations were performed with bead-coupled SRRM2-specific antibodies 13F11, 18A4-2 and 23A7 and lysates of transfected cells or, as control, non-transfected 293 cells (HEK293). Precipitated proteins were separated by PAGE and transferred to a PVDF membrane, which was then incubated with an anti-rat antibody coupled to HRP. Finally, the blot was developed with ECL. As additional controls, lysates from transfected and non-transfected cells were included.

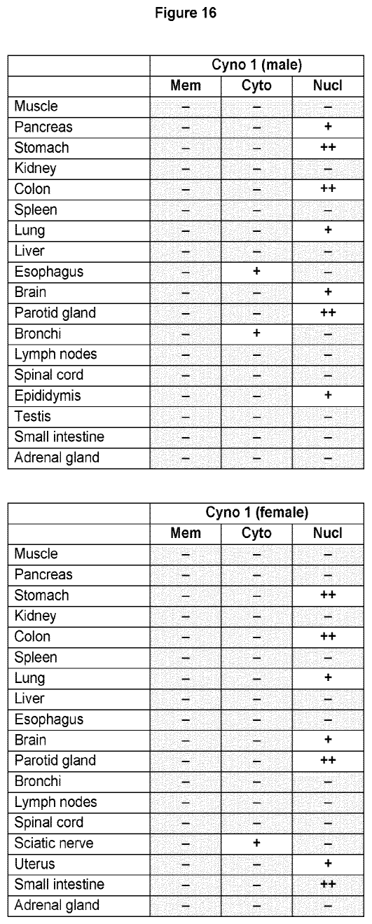

Example 15 shows that normal tissues from Cynomolgus monkey either stain negative for SRRM2 or show cytoplasmic/nuclear staining (see FIG. 16) as revealed by immunohistochemistry. No membrane staining was observed. Mem=membrane; Cyto=cytoplasmic; Nucl=nuclear. FFPE tissues from cynomolgus monkey were incubated with 13F11 antibody, followed by incubation with a goat anti-rat IgG antibody coupled to HRP (ImmPRESS HRP Peroxidase detection kit; https://vectorlabs.com/products/enzyme-polymer/immpress-hrp-goat-anti-rat-igg-kit).

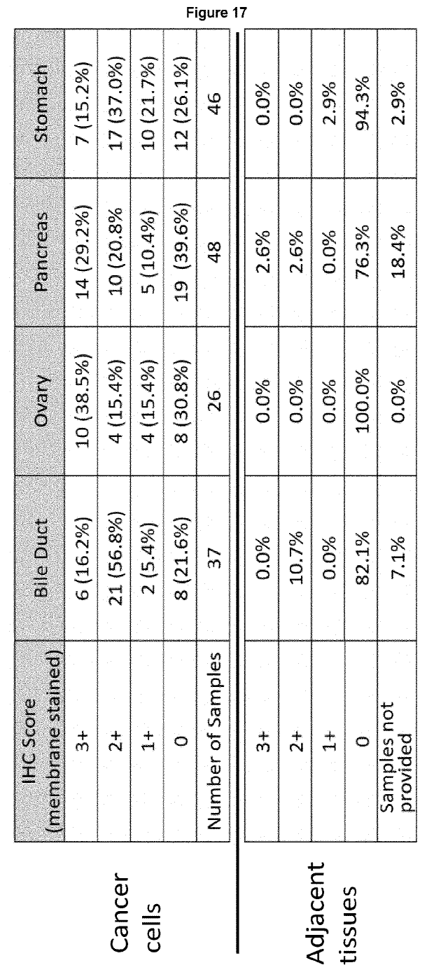

Example 16 shows that the majority of tumor samples derived from different locations (bile duct, ovary, pancreas, stomach) reveal SRRM2 membrane staining (see FIG. 17). Formalin-fixed paraffin embedded (FFPE) tumor tissues and adjacent normal tissues were stained with 13F11, followed by incubation with a rat-specific secondary antibody coupled with HRP. Tissues were inspected by a trained pathologist and staining intensity was scored from 0 (negative) to 3+ (strong expression on the majority of cells).

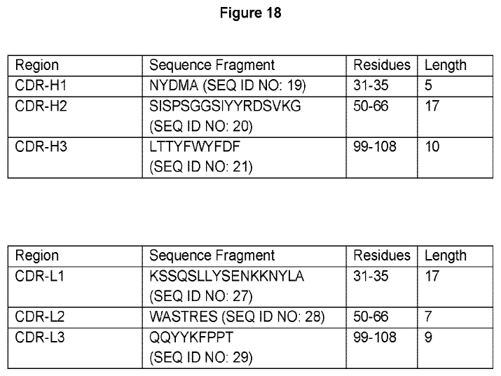

Example 17 shows the sequences of the light and heavy chains and sequences of its fragments of antibody 18A4-2 (see FIG. 18).

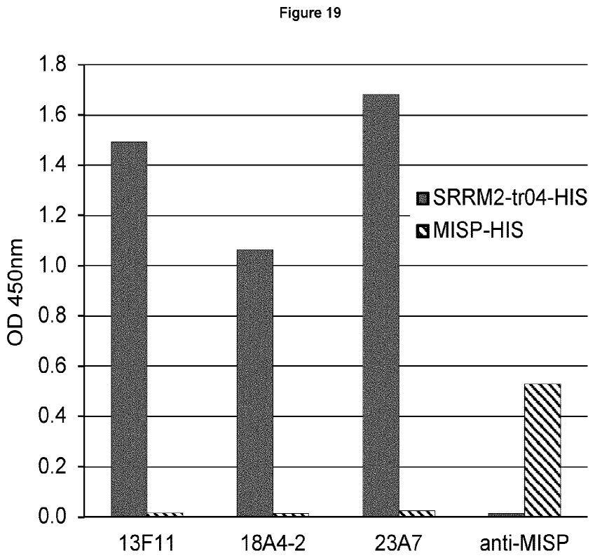

Example 18 shows that the SRRM2-specific antibodies 13F11, 18A4-2 and 23A7 bind to fragment tr04 (see FIG. 19). A 96-well cell cluster plate was coated with purified SRRM2-tr04-HIS or, as a control, MISP-HIS protein (each at 50 μg/ml) overnight and then blocked with non-fat milk powder in TBST. Antibodies 13F11, 18A4-2, 23A7 or an anti-MISP antibody were added for 2 hours at room temperature. After washing, the plate was incubated with a secondary anti-rat IgG antibody coupled to HRP and developed with TMB. After stopping the reaction with H₂SO₄, the absorbance was measured at 450 nm.

### REFERENCES

Hashimoto-Gotoh, T. et al. (1995) Gene 152, 271-275.
Kramer, W. et al. (1984) Nucleic Acids Res. 12, 9441-9456.
Kunkel, TA (1985) Proc Natl Acad Sci USA. 82, 488-492.
Kunkel (1988) Methods Enzymol. 85, 2763-2766.
Mark, D. F. et al., Proc. Natl. Acad. Sci. USA (1984) 81, 5662-5666, Zoller, M. J. and Smith, M., Nucleic Acids Research (1982) 10, 6487-6500.
Wang, A. et al., Science 224, 1431-1433, Dalbadie-McFarland, G. et al., Proc. Natl. Acad. Sci. USA (1982) 79, 6409-641.
Weidle, U. H., Maisel, D., Klostermann, S., Schiller, C., and Weiss, E. H. (2011). Intracellular proteins displayed on the surface of tumor cells as targets for therapeutic intervention with antibody-related agents. Cancer Genomics Proteomics 8, 49-63.
Zoller, M J, and Smith, M. (1983) Methods Enzymol. 100, 468-500.

1. A pharmaceutical composition comprising an antibody, which binds to human Serine/arginine repetitive matrix protein 2 (SRRM2) present on the cell surface of a target cell.

2. The pharmaceutical composition of claim 1, wherein said antibody has cytotoxic activity.

3. The pharmaceutical composition of claim 1, wherein said antibody has antibody-dependent cell-meditated cytotoxicity (ADCC) or complement-dependent cytotoxicity (CDC).

4. The pharmaceutical composition of claim 1, wherein said antibody is conjugated with a cytotoxic substance.

5. The pharmaceutical composition of claim 1, wherein said antibody is part of a chimeric antigen receptor (CAR).

6. (canceled)

7. The pharmaceutical composition of claim 1, wherein said SRRM2 present on the cell surface of a target cell is externalized.

8. The pharmaceutical composition of claim 1, wherein the antibody is not an intracellular antibody.

9. The pharmaceutical composition of claim 1, wherein said antibody is
(a) an antibody comprising a heavy chain variable region having an amino acid sequence with at least 85%, 90% or 95% identity to the amino acid sequence shown in SEQ ID NO: 1 and a light chain variable region having an amino acid sequence with at least 85%, 90% or 95% identity to the amino acid sequence shown in SEQ ID NO: 2;
(b) an antibody comprising a heavy chain variable region having an amino acid sequence with at least 85%, 90% or 95% identity to the amino acid sequence shown in SEQ ID NO: 3 and a light chain variable region having an amino acid sequence with at least 85%, 90% or 95% identity to the amino acid sequence shown in SEQ ID NO: 4;
(c) an antibody comprising a heavy chain variable region having an amino acid sequence with at least 85%, 90% or 95% identity to the amino acid sequence shown in SEQ ID NO: 5 and a light chain variable region having an amino acid sequence with at least 85%, 90% or 95% identity to the amino acid sequence shown in SEQ ID NO: 6;
(d) an antibody comprising a heavy chain variable region having an amino acid sequence with at least 85%, 90% or 95% identity to the amino acid sequence shown in SEQ ID NO: 5 and a light chain variable region having an amino acid sequence with at least 85%, 90% or 95% identity to the amino acid sequence shown in SEQ ID NO: 26; or
(e) an antibody, which binds to the same epitope as that in the human SRRM2 protein to which any one of the antibodies (a) to (d) bind.

10. The pharmaceutical composition of claim 1, wherein said antibody is
(a) an antibody comprising a heavy chain variable region comprising heavy chain CDR1 having the amino acid sequence as set forth in SEQ ID NO: 7, heavy chain CDR2 having the amino acid sequence as set forth in SEQ ID NO: 8, and heavy chain CDR3 having the amino acid sequence as set forth in SEQ ID NO: 9, and
a light chain variable region comprising light chain CDR1 having the amino acid sequence as set forth in SEQ ID NO: 10, light chain CDR2 having the amino acid sequence as set forth in SEQ ID NO: 11, and light chain CDR3 having the amino acid sequence as set forth in SEQ ID NO: 12;
(b) an antibody comprising a heavy chain variable region comprising heavy chain CDR1 having the amino acid sequence as set forth in SEQ ID NO: 13, heavy chain CDR2 having the amino acid sequence as set forth in SEQ ID NO: 14, and heavy chain CDR3 having the amino acid sequence as set forth in SEQ ID NO: 15, and
a light chain variable region comprising light chain CDR1 having the amino acid sequence as set forth in SEQ ID NO: 16, light chain CDR2 having the amino acid sequence as set forth in SEQ ID NO: 17, and light chain CDR3 having the amino acid sequence as set forth in SEQ ID NO: 18;
(c) an antibody comprising a heavy chain variable region comprising heavy chain CDR1 having the amino acid sequence as set forth in SEQ ID NO: 19, heavy chain CDR2 having the amino acid sequence as set forth in SEQ ID NO: 20, and heavy chain CDR3 having the amino acid sequence as set forth in SEQ ID NO: 21, and
a light chain variable region comprising light chain CDR1 having the amino acid sequence as set forth in SEQ ID NO: 22, light chain CDR2 having the amino acid sequence as set forth in SEQ ID NO: 23, and light chain CDR3 having the amino acid sequence as set forth in SEQ ID NO: 24;
(d) an antibody comprising a heavy chain variable region comprising heavy chain CDR1 having the amino acid sequence as set forth in SEQ ID NO: 19, heavy chain CDR2 having the amino acid sequence as set forth in SEQ ID NO: 20, and heavy chain CDR3 having the amino acid sequence as set forth in SEQ ID NO: 21, and
a light chain variable region comprising light chain CDR1 having the amino acid sequence as set forth in SEQ ID NO: 27, light chain CDR2 having the amino acid sequence as set forth in SEQ ID NO: 28, and light chain CDR3 having the amino acid sequence as set forth in SEQ ID NO: 29; or
(e) an antibody, which binds to the same epitope as that in the human SRRM2 protein to which any one of the antibodies (a) to (d) bind.

11. A method of treating cancer comprising the step of administering a therapeutically effective amount of the pharmaceutical composition of claim 1 to a human subject in need thereof.

12. The method of claim 11, wherein said cancer is breast cancer, prostate cancer, lung cancer, colorectal cancer, ovarian cancer, pancreatic cancer, stomach cancer, cholangiogenic cancer, oral cavity squamous carcinoma, hematologic malignancy tumor, or glioma.

13. (canceled)

14. (canceled)

15. An antibody, which binds to human SRRM2 present on the cell surface of a target cell, comprising
(a) an antibody comprising a heavy chain variable region comprising heavy chain CDR1 having the amino acid sequence as set forth in SEQ ID NO: 7, heavy chain CDR2 having the amino acid sequence as set forth in SEQ ID NO: 8, and heavy chain CDR3 having the amino acid sequence as set forth in SEQ ID NO: 9, and
a light chain variable region comprising light chain CDR1 having the amino acid sequence as set forth in SEQ ID NO: 10, light chain CDR2 having the amino acid sequence as set forth in SEQ ID NO: 11, and light chain CDR3 having the amino acid sequence as set forth in SEQ ID NO: 12;
(b) an antibody comprising a heavy chain variable region comprising heavy chain CDR1 having the amino acid sequence as set forth in SEQ ID NO: 13, heavy chain CDR2 having the amino acid sequence as set forth in SEQ ID NO: 14, and heavy chain CDR3 having the amino acid sequence as set forth in SEQ ID NO: 15, and
a light chain variable region comprising light chain CDR1 having the amino acid sequence as set forth in SEQ ID NO: 16, light chain CDR2 having the amino acid sequence as set forth in SEQ ID NO: 17, and light chain CDR3 having the amino acid sequence as set forth in SEQ ID NO: 18;
(c) an antibody comprising a heavy chain variable region comprising heavy chain CDR1 having the amino acid sequence as set forth in SEQ ID NO: 19, heavy chain CDR2 having the amino acid sequence as set forth in SEQ ID NO: 20, and heavy chain CDR3 having the amino acid sequence as set forth in SEQ ID NO: 21, and
a light chain variable region comprising light chain CDR1 having the amino acid sequence as set forth in SEQ ID NO: 22, light chain CDR2 having the amino acid sequence as set forth in SEQ ID NO: 23, and light chain CDR3 having the amino acid sequence as set forth in SEQ ID NO: 24;
(d) an antibody comprising a heavy chain variable region comprising heavy chain CDR1 having the amino acid sequence as set forth in SEQ ID NO: 19, heavy chain CDR2 having the amino acid sequence as set forth in SEQ ID NO: 20, and heavy chain CDR3 having the amino acid sequence as set forth in SEQ ID NO: 21, and
a light chain variable region comprising light chain CDR1 having the amino acid sequence as set forth in SEQ ID NO: 27, light chain CDR2 having the amino acid sequence as set forth in SEQ ID NO: 28, and light chain CDR3 having the amino acid sequence as set forth in SEQ ID NO: 29; or
(e) an antibody, which binds to the same epitope as that in the human SRRM2 protein to which any one of the antibodies (a) to (d) bind.

16. (canceled)

17. (canceled)

18. (canceled)

19. A method of killing a target cell having SRRM2 present on the cell surface of said target cell, comprising administering a therapeutically effective amount of the antibody of claim 15 to a subject in need thereof, wherein the antibody which binds to human SRRM2 present on the cell surface of said target cell.

20. (canceled)

21. (canceled)

22. The method of claim 11, wherein the pharmaceutical composition comprises an antibody, which binds to human SRRM2 present on the cell surface of a target cell.

23. The method of claim 22, wherein said antibody is
(a) an antibody comprising a heavy chain variable region having an amino acid sequence with at least 85%, 90% or 95% identity to the amino acid sequence shown in SEQ ID NO: 1 and a light chain variable region having an amino acid sequence with at least 85%, 90% or 95% identity to the amino acid sequence shown in SEQ ID NO: 2;
(b) an antibody comprising a heavy chain variable region having an amino acid sequence with at least 85%, 90% or 95% identity to the amino acid sequence shown in SEQ ID NO: 3 and a light chain variable region having an amino acid sequence with at least 85%, 90% or 95% identity to the amino acid sequence shown in SEQ ID NO: 4;
(c) an antibody comprising a heavy chain variable region having an amino acid sequence with at least 85%, 90% or 95% identity to the amino acid sequence shown in SEQ ID NO: 5 and a light chain variable region having an amino acid sequence with at least 85%, 90% or 95% identity to the amino acid sequence shown in SEQ ID NO: 6;
(d) an antibody comprising a heavy chain variable region having an amino acid sequence with at least 85%, 90% or 95% identity to the amino acid sequence shown in SEQ ID NO: 5 and a light chain variable region having an amino acid sequence with at least 85%, 90% or 95% identity to the amino acid sequence shown in SEQ ID NO: 26;
(e) an antibody, which binds to the same epitope as that in the human SRRM2 protein to which any one of the antibodies (a) to (d) bind.

24. The method of claim 22, wherein said antibody is
(a) an antibody comprising a heavy chain variable region comprising heavy chain CDR1 having the amino acid sequence as set forth in SEQ ID NO: 7, heavy chain CDR2 having the amino acid sequence as set forth in SEQ ID NO: 8, and heavy chain CDR3 having the amino acid sequence as set forth in SEQ ID NO: 9, and
a light chain variable region comprising light chain CDR1 having the amino acid sequence as set forth in SEQ ID NO: 10, light chain CDR2 having the amino acid sequence as set forth in SEQ ID NO: 11, and light chain CDR3 having the amino acid sequence as set forth in SEQ ID NO: 12;
(b) an antibody comprising a heavy chain variable region comprising heavy chain CDR1 having the amino acid sequence as set forth in SEQ ID NO: 13, heavy chain CDR2 having the amino acid sequence as set forth in SEQ ID NO: 14, and heavy chain CDR3 having the amino acid sequence as set forth in SEQ ID NO: 15, and
a light chain variable region comprising light chain CDR1 having the amino acid sequence as set forth in SEQ ID NO: 16, light chain CDR2 having the amino acid sequence as set forth in SEQ ID NO: 17, and light chain CDR3 having the amino acid sequence as set forth in SEQ ID NO: 18;
(c) an antibody comprising a heavy chain variable region comprising heavy chain CDR1 having the amino acid sequence as set forth in SEQ ID NO: 19, heavy chain CDR2 having the amino acid sequence as set forth in SEQ ID NO: 20, and heavy chain CDR3 having the amino acid sequence as set forth in SEQ ID NO: 21, and
a light chain variable region comprising light chain CDR1 having the amino acid sequence as set forth in SEQ ID NO: 22, light chain CDR2 having the amino acid sequence as set forth in SEQ ID NO: 23, and light chain CDR3 having the amino acid sequence as set forth in SEQ ID NO: 24;
(d) an antibody comprising a heavy chain variable region comprising heavy chain CDR1 having the amino acid sequence as set forth in SEQ ID NO: 19, heavy chain CDR2 having the amino acid sequence as set forth in SEQ ID NO: 20, and heavy chain CDR3 having the amino acid sequence as set forth in SEQ ID NO: 21, and
a light chain variable region comprising light chain CDR1 having the amino acid sequence as set forth in SEQ ID NO: 27, light chain CDR2 having the amino acid sequence as set forth in SEQ ID NO: 28, and light chain CDR3 having the amino acid sequence as set forth in SEQ ID NO: 29; or
(e) an antibody, which binds to the same epitope as that in the human SRRM2 protein to which any one of the antibodies (a) to (d) bind.

25. The pharmaceutical composition of claim 1, wherein the antibody is a monoclonal antibody.

26. The pharmaceutical composition of claim 1, comprising a pharmaceutically acceptable carrier, diluent or excipient.

27. The method of treating of claim 11, wherein said cancer comprises cells having a cell surface, wherein human SRRM2 is present on the cell surface.

28. The antibody of claim 15, wherein said antibody is
(a) an antibody comprising a heavy chain variable region having an amino acid sequence with at least 85%, 90% or 95% identity to the amino acid sequence shown in SEQ ID NO: 1 and a light chain variable region having an amino acid sequence with at least 85%, 90% or 95% identity to the amino acid sequence shown in SEQ ID NO: 2;
(b) an antibody comprising a heavy chain variable region having an amino acid sequence with at least 85%, 90% or 95% identity to the amino acid sequence shown in SEQ ID NO: 3 and a light chain variable region having an amino acid sequence with at least 85%, 90% or 95% identity to the amino acid sequence shown in SEQ ID NO: 4;
(c) an antibody comprising a heavy chain variable region having an amino acid sequence with at least 85%, 90% or 95% identity to the amino acid sequence shown in SEQ ID NO: 5 and a light chain variable region having an amino acid sequence with at least 85%, 90% or 95% identity to the amino acid sequence shown in SEQ ID NO: 6;
(d) an antibody comprising a heavy chain variable region having an amino acid sequence with at least 85%, 90% or 95% identity to the amino acid sequence shown in SEQ ID NO: 5 and a light chain variable region having an amino acid sequence with at least 85%, 90% or 95% identity to the amino acid sequence shown in SEQ ID NO: 26; or
(e) an antibody, which binds to the same epitope as that in the human SRRM2 protein to which any one of the antibodies (a) to (d) bind.
